(19) United States
(12) Patent Application Publication
(10) Pub. No.: US 2020/0385370 A1
(43) Pub. Date: Dec. 10, 2020

(54) APPARATUS FOR REMOVING BAD ODOR FROM TOILET

(71) Applicant: SYNBLIA THERAPEUTICS, INC., Dover, DE (US)

(72) Inventors: Yinong XIE, Dover, DE (US); Lee E BABISS, Dover, DE (US)

(21) Appl. No.: 16/768,503

(22) PCT Filed: Oct. 29, 2018
(86) PCT No.: PCT/US2018/057936

§ 371 Date: May 29, 2020

Related U.S. Application Data
(60) Provisional application No. 62/578,617, filed on Oct. 30, 2017.

(51) Int. Cl. C07D 413/12; C07D 401/12; C07D 401/04; C07D 209/08

(52) U.S. Cl. CPC C07D 413/12; C07D 209/08; C07D 401/04; C07D 401/12

(57) ABSTRACT

Compounds of Formula I as IRAK4 inhibitors are disclosed. The pharmaceutical compositions comprising compounds of formula I, methods of synthesis of these compounds, methods of treatment for diseases associated with IRAK-4 such as inflammatory diseases and autoimmune diseases using these compounds or compositions containing these compounds are also disclosed.

### CROSS-REFERENCE TO RELATED APPLICATIONS

This application claims the benefit of U.S. Provisional Application No. 62/578,617, filed Oct. 30, 2017; which is incorporated by reference by its entirety.

### FIELD

The present invention pertains to compounds as Interleukin-1 Receptor Associated Kinase 4 (IRAK4) modulators and their use in the treatment of, but not limited to, cancers, autoimmune, inflammatory diseases, and autoinflammatory conditions related to IRAK4 overexpression.

### BACKGROUND

Protein kinases are families of enzymes that catalyze the phosphorylation of specific residues in proteins. In general, they are classified in the groups of tyrosine and serine/threonine kinases. Inappropriate activity from dysregulation of certain kinases is believed to be underlying causes of many diseases, including, but not limited to, cancer, cardiovascular diseases, allergies, asthma, respiratory diseases, autoimmune diseases, inflammatory diseases, bone diseases, metabolic disorders, and neurological and neurodegenerative diseases.

Members of the Interleukin-1 receptor associated kinase (IRAK) family are protein kinase targets of particular interest for the development of anti-tumor, autoimmune and anti-inflammatory drugs, especially IRAK4.

IRAK4 has been recognized as an important pharmacological target for the treatment of chronic inflammatory diseases. It is a ubiquitously expressed serine/threonine kinase involved in the innate inflammatory signaling directly downstream of the Toll like receptors (TLRs) and interleukin-1 (IL-1) family of receptors. TLRs represent a first line of defense against pathogens such as bacteria, viruses, and yeast. The IL-1 family of receptors also plays important roles in the immediate inflammatory response to invading organisms. In addition, IRAK4 is expressed in T and B lymphocytes and has been reported to play an important role in cross talk between the innate and adaptive immune systems. IRAK4 kinase-dead knock-in mice have shown to be resistant to induced joint inflammation in the antigen-induced-arthritis (AIA) and serum transfer-induced (K/BxN) arthritis models. Likewise, humans deficient in IRAK4 also appear to display impaired activation of the innate immune response but no increased susceptibility to viral or fungal infection and only increased infection risk by a narrow range of pyogenic bacteria prior to adolescence.

These research results suggest that selective small molecule inhibitors of IRAK4 may have therapeutic value in treating cytokine driven autoimmune diseases while avoiding broad immunosuppression side effects. Additionally, recent studies indicate that targeting IRAK4 may be useful in other inflammatory pathologies such as atherosclerosis and diffuse large B-cell lymphoma. Therefore, inhibitors of IRAK4 kinase activity are potential therapeutics for a wide variety of diseases such as autoimmunity, inflammation, cardiovascular diseases, cancer, and metabolic diseases.

### SUMMARY

This disclosure relates to compounds represented by Formula 1:

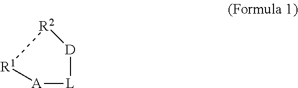

or a pharmaceutically acceptable salt thereof; wherein a dashed line indicates the presence or absence of a covalent bond; A is an optionally substituted fused bicyclic heteroaryl group, an optionally substituted naphthyl group, or an optionally substituted fused tricyclic heteroaryl group, wherein A contains a R¹ substituent; L is a direct covalent bond, optionally substituted C₁₋₃H₂₋₆X₀₋₁, or X, wherein X is O, S, SO, SO₂, or NH; D is an optionally substituted heterocyclic ring, or an optionally substituted fused or spiro bicyclic group; R¹ is H, —NR^{A}R^{B}, —OR^{A}, —O—R^{A}—O—R^{B}, —O—R^{A}—O—R^{B}—O—R^{C}, —C(O)NR^{A}R^{B}, or SR^{A}; R² is H, —C(O)— or a direct covalent bond to R¹; and R^{A}, R^{B}, R^{C}, and R^{D} are independently H or C₁₋₁₂ hydrocarbyl. In some embodiments, A-L is A-S(O)₀₋₂C(R^{A})(R^{B})—, A-OC(R^{A})(R^{B})—, A-N(R^{C})C(R^{A})(R^{B}), A-S(O)₀₋₂C(R^{A})(R^{B})—,

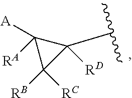

A-C(R^{A})(R^{B})C(R^{C})(R^{D})—, A-N(R^{C})—, or A-S(O)₀₋₂, or L is a covalent bond. In some embodiments, A contains an optionally substituted aromatic all carbon ring which attaches to R¹.

Some embodiments include a method of treating cancer, autoimmune diseases, inflammatory diseases, autoinflammatory conditions, and other IRAK4-mediated disorders in a mammal comprising administering a compound described herein, or any optionally substituted compound represented in Table I below, or a pharmaceutically acceptable salt thereof (referred to collectively herein as a “subject compound”), to a patient in need thereof.

Some embodiments include use of a compound described herein, such as a compound of Formula 1, a subject compound described herein in the manufacture of a medicament for the treatment of cancer, autoimmune diseases, inflammatory diseases, autoinflammatory conditions, and other IRAK4-mediated disorders in a mammal.

Some embodiments include a pharmaceutical composition comprising a therapeutically effective amount of a subject compound described herein in combination with at least one pharmaceutically acceptable carrier.

Some embodiments include a process for making a pharmaceutical composition comprising combining a subject compound described herein and at least one pharmaceutically acceptable carrier.

### DETAILED DESCRIPTION

Unless otherwise indicated, any reference to a compound herein by structure, name, or any other means, includes pharmaceutically acceptable salts, such as sodium, potassium, and ammonium salts, or HCl, H₂SO₄, HCO₂H, and CF₃CO₂H salts; prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

If stereochemistry is not indicated, a name or structural depiction includes any stereoisomer or any mixture of stereoisomers.

In some embodiments, a compound of Formula 1 is an R-enantiomer. In some embodiments, a compound of Formula 1 is an S-enantiomer.

Unless otherwise indicated, when a compound or chemical structural feature such as aryl is referred to as being “optionally substituted,” it includes a feature that has no substituents (i.e. unsubstituted), or a feature that is “substituted,” meaning that the feature has one or more substituents. The term “substituent” is broad, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of 15 g/mol to 50 g/mol, 15 g/mol to 100 g/mol, 15 g/mol to 150 g/mol, 15 g/mol to 200 g/mol, 15 g/mol to 300 g/mol, or 15 g/mol to 500 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes at least one C, N, O, S, P, Si, F, C, Br, or I atom. Examples of substituents include, but are not limited to, alkyl, alkenyl, alkynyl, heteroalkyl, heteroalkenyl, heteroalkynyl, aryl, heteroaryl, hydroxy, alkoxy, aryloxy, acyl, acyloxy, alkylcarboxylate, thiol, alkylthio, cyano, halo, thiocarbonyl, O-carbamyl, N-carbamyl, O-thiocarbamyl, N-thiocarbamyl, C-amido, N-amido, S-sulfonamido, N-sulfonamido, N-oxide, isocyanato, thiocyanato, isothiocyanato, nitro, silyl, sulfenyl, sulfinyl, sulfonyl, sulfoxide, haloalkyl, haloalkoxyl, trihalomethanesulfonyl, trihalomethanesulfonamido, amino, phosphonic acid, etc.

For convenience, the term “molecular weight” is used with respect to a moiety or part of a molecule to indicate the sum of the atomic masses of the atoms in the moiety or part of a molecule, even though it may not be a complete molecule.

A hydrogen atom in any position of a compound of Formula 1 may be replaced by a deuterium. In some embodiments, a compound of Formula 1 contains a deuterium atom or multiple deuterium atoms.

With respect to any relevant structural representation, R^{A}, R^{B}, R^{C}, and R^{D} may be independently H or C₁₋₁₂ hydrocarbyl, such as C₁₋₁₂ alkyl, C₁₋₁₂ alkenyl, C₁₋₁₂ alkynyl, phenyl, etc., including: linear or branched alkyl having a formula CₐH₂ₐ₊₁, or cycloalkyl having a formula CₐH₂ₐ₋₁, wherein a is 1, 2, 3, 4, 5, 6, 7, 8, 9, 10, 11, or 12, such as linear or branched alkyl with a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl with a formula: C₃H₅, C₄H₇, C₅H₉, C₆H₁₁, C₇H₁₃, C₈H₁₅, C₉H₁₇, C₁₀H₁₉, etc. In some embodiments, R^{A}, R^{B}, R^{C}, and R^{D} may be independently H or C₁₋₆ alkyl. In some embodiments, R^{A}, R^{B}, R^{C}, and R^{D} may be independently H or C₁₋₃ alkyl. In some embodiments, R^{A}, R^{B}, R^{C}, and R^{D} may be independently H or CH₃. In some embodiments, R^{A}, R^{B}, R^{C}, and R^{D} may be independently H.

With respect to Formula 1, in some embodiments, A contains a R¹ substituent. In some embodiments, A contains an optionally substituted aromatic all carbon ring. In some embodiments, the aromatic all carbon ring attaches to R¹. In some embodiments, A contains a R¹ substituent, and an optionally substituted aromatic all carbon ring.

With respect to Formula 1, in some embodiments, A is: optionally substituted 2-oxo-2,3-dihydro-1H-imidazo[4,5-g]isoquinolin-4-yl; optionally substituted isoquinolinyl; optionally substituted quinolinyl; optionally substituted naphthyl; optionally substituted quinazoline, optionally substituted 1,3-dioxo-2,3-dihydro-1H-pyrrolo[3,4-g]isoquinolinyl; optionally substituted 3-imino-2-oxo-2,3-dihydro-1H-pyrrolo[2,3-g]isoquinolinyl; 3-(hydroxyimino)-2-oxo-2,3-dihydro-1H-pyrrolo[2,3-g]isoquinolin-yl; optionally substituted indolyl; optionally substituted benzoimidazolyl; optionally substituted 1H-imidazo[1,2-a]indolyl; optionally substituted naphtho[2,3-b]thiophenyl; optionally substituted thiazolo[3,2-a]indolyl; optionally substituted 1-H-benzo[f]indolyl; optionally substituted 2-oxo-1,2-dihydrobenzo[g]quinoxalinyl; optionally substituted 2-oxo-1,2,3,4-tetrahydrobenzo[g]quinoxalinyl; optionally substituted naphtho[2,3-b]furanyl; optionally substituted oxazolo[3,2-a]indolyl, optionally substituted 3H-imidazo[4,5-c]isoquinolin-2-amine, optionally substituted thiazolo[5,4-c]isoquinolin-2-amine, or optionally substituted oxazolo[5,4-c]isoquinolin-2-amine.

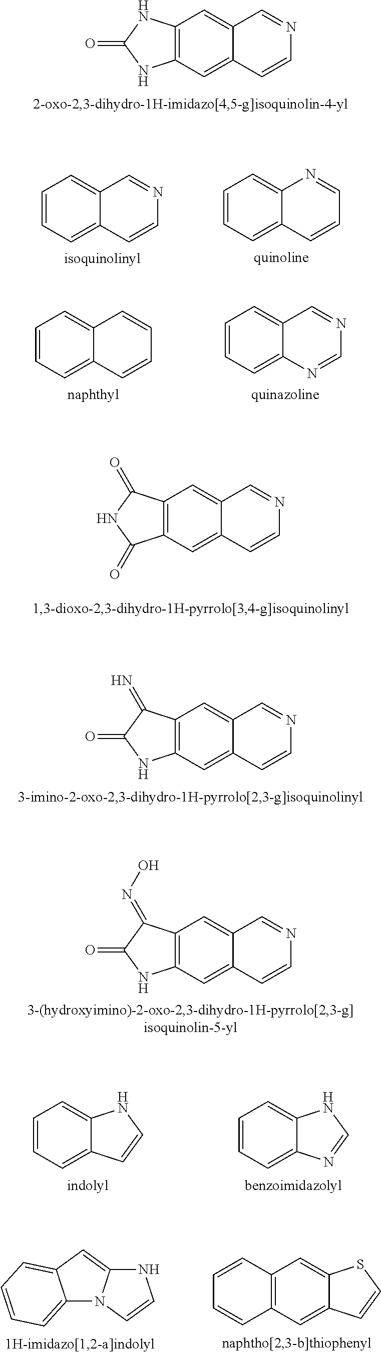

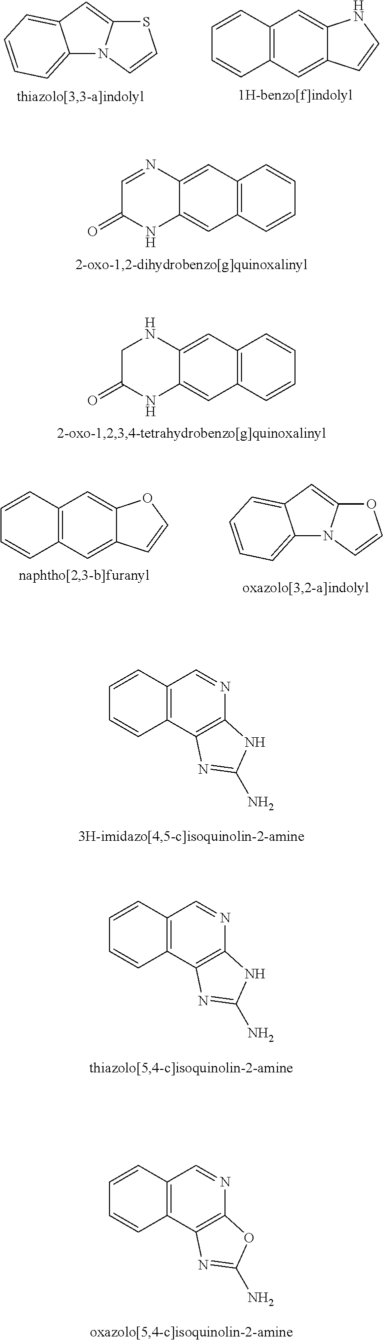

In some embodiments, A has an —OC(O)NH₂ substituent.

In some embodiments, A has a —C(O)NH₂ substituent.

In some embodiments, A has a —C(O)NHOH substituent.

In some embodiments, A has a —C(O)NHS(O)₂CH₃ substituent.

In some embodiments, A has a —C(O)NHCN substituent.

In some embodiments, A has an —OH substituent.

In some embodiments, A has a —C(O)CHF₂ substituent.

In some embodiments, A has an —NHC(O)CH₃ substituent.

In some embodiments, A has an —NH₂ substituent.

In some embodiments, A has a —C(S)NH₂ substituent.

In some embodiments, A has an —SC(O)NH₂ substituent.

In some embodiments, A has an —OC(S)NH₂ substituent.

In some embodiments, A has an —NHC(S)NH₂ substituent.

In some embodiments, A has a —C(O)SH substituent.

In some embodiments, A has an —NHC(═NCH₃)NH₂ substituent.

In some embodiments, A has an —NHC(O)SCH₃ substituent.

In some embodiments, A has an —NHC(O)OCH₃ substituent.

In some embodiments, A has a —C≡C—CH₃ substituent.

In some embodiments, A has a —Br substituent.

In some embodiments, A has a —CN substituent.

In some embodiments, A is optionally substituted isoquinolinyl, wherein 2 or more of the substituents may be connected (e.g. as shown in the dashed lines) to form a further fused ring with the isoquinolinyl. For example, in some embodiments, R¹-A is represented by Formula A1a or A1b:

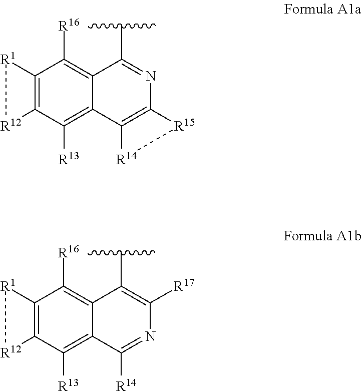

In some embodiments, A is optionally substituted indolyl, wherein 2 or more of the substituents may be connected (e.g. as shown in the dashed lines) to form a further fused ring with the indolyl. For example, in some embodiments, R¹-A is represented by Formula A2:

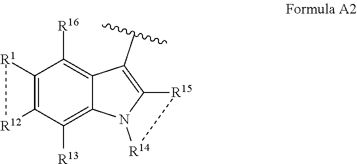

In some embodiments, A is optionally substituted naphthyl, wherein 2 or more of the substituents may be connected (e.g. as shown in the dashed lines) to form a further fused ring with the naphthyl. For example, in some embodiments, R¹-A is represented by Formula A3:

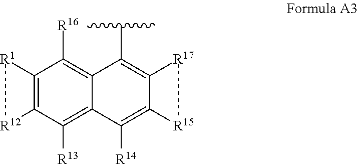

In some embodiments, A is optionally substituted quinoline, wherein 2 or more of the substituents may be connected (e.g. as shown in the dashed lines) to form a further fused ring with quinoline. For example, in some embodiments, R¹-A is represented by Formula A4:

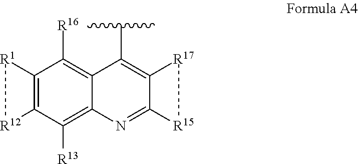

In some embodiments, A is optionally substituted quinazoline, wherein 2 or more of the substituents may be connected (e.g. as shown in the dashed lines) to form a further fused ring with quinazoline. For example, in some embodiments, R¹-A is represented by Formula A5:

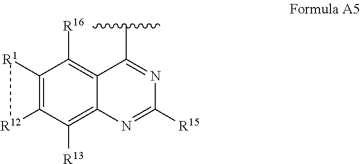

With respect to any relevant structural representation, such as Formula A1a, A1b, A2, A3, A4, or A5, R¹², R¹³, R¹⁵, R¹⁶, and R¹⁷ are independently H or any substituent, such as a substituent having a molecular weight of 15-200 Da, 15-150 Da, 15-100 Da, or 15-50 Da. In some embodiments R¹², R¹³, R¹⁵, R¹⁶, and R¹⁷ are independently H; F; Cl; Br; I; C₁₋₆H₀₋₁₆N₀₋₃O₀₋₃F₀₋₃; C₀₋₃N₁₋₃O₀₋₃H₀₋₁₀; or C₀₋₃N₀₋₃O₁₋₃H₀₋₁₀. In some embodiments R¹², R¹³, R¹⁵, R¹⁶, and R¹⁷ are independently H, C₁₋₃ alkyl, F, Cl, Br, or CN.

With respect to any relevant structural representation, such as Formula A1a, A1b, A2, or A3, R¹⁴ is independently H or any substituent, such as a substituent having a molecular weight of 15-200 Da, 15-150 Da, 15-100 Da, or 15-50 Da. In some embodiments R¹⁴ is independently H; F; Cl; Br; I; C₁₋₆H₀₋₁₆N₀₋₃O₀₋₃F₀₋₃; C₀₋₃N₁₋₃H₀₋₁₀; or C₀₋₃N₀₋₃O₁₋₃H₀₋₁₀. In some embodiments, R¹⁴ is independently H, C₁₋₃ alkyl, F, Cl, Br, CN (except in Formula A2), —C≡C—R wherein R is H or C₁₋₃ alkyl, —(CH₂)ₙNR^{A}R^{B}, —(CH₂)ₙ—C₆₋₁₀ aryl, or —(CH₂)ₙ-(5 to 10 membered heteroaryl having 1, 2, or 3 heteroatoms selected from N, O, or S), wherein said aryl or heteroaryl is optionally substituted by one, two, or three C₁₋₆ alkyl, deuterium, halogen, CN, OH, or C₁₋₆alkoxy group, or any combination thereof.

With respect to any relevant structural representation, such as Formula A1, A2, A3, or A4, in some embodiments R¹² is —C(═O)NH—R^{E}, wherein R^{E} is H, or a substituent with a molecular weight less than 50 Da, such as —OH.

With respect to any relevant structural representation, such as Formula A1, A2, A3, or A4, in some embodiments, R¹³ is H.

With respect to any relevant structural representation, such as Formula A1, A2, A3, or A4, in some embodiments, R¹⁴ is H. In some embodiments, R¹⁴ is —C≡C—CH₃. In some embodiments, R¹⁴ is —Br. In some embodiments, R¹⁴ is —CN.

With respect to any relevant structural representation, such as Formula A1, A2, A3, or A4, in some embodiments, R¹⁵ is H.

With respect to any relevant structural representation, such as Formula A1, A2, A3, or A4, in some embodiments, R¹⁶ is H.

With respect to any relevant structural representation, such as Formula A3, R¹⁷ is H or any substituent, such as a substituent having a molecular weight of 15-200 Da, 15-150 Da, 15-100 Da, or 15-50 Da. In some embodiments, R¹⁷ is H; F; Cl; Br; I; C₁₋₆H₀₋₁₆N₀₋₃O₀₋₃F₀₋₃; C₀₋₃N₁₋₃O₀₋₃H₀₋₁₀; or C₀₋₃N₀₋₃O₁₋₃H₀₋₁₀. In some embodiments R¹⁷ is H.

With respect to Formula 1, in some embodiments D is: optionally substituted 5-oxopyrrolidinyl; optionally substituted 2-oxooxazolidinyl; optionally substituted 2-oxoimidazolidinyl; optionally substituted octahydrocyclopenta[c]pyrrolyl; optionally substituted azetidinyl; optionally substituted 4-oxohexahydropyrrolo[3,4-c]pyrrol-2(1H)-yl, optionally substituted piperidine, optionally substituted cyclopentane, optionally substituted piperazine, optionally substituted 1H-1,2,3-triazole, optionally substituted 2-oxa-8-azaspiro[4.5]decane, or optionally substituted pyrrolidine.

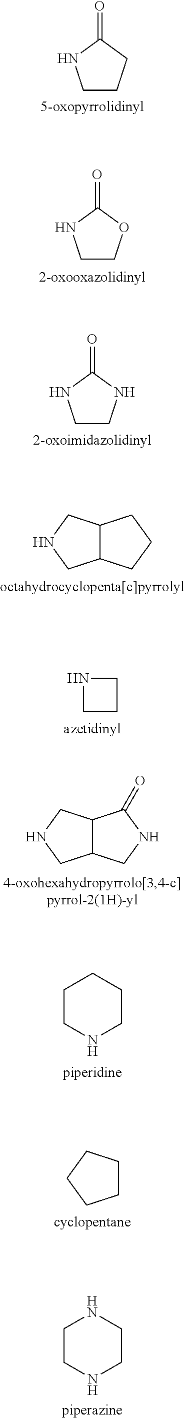

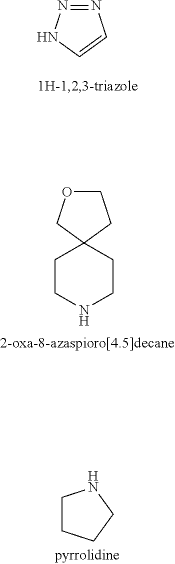 [SMILES: c1c[nH]nn1 ; C1CC2(CCN1)CCOC2 ; C1CCNC1]

In some embodiments, D has an —NH₂ substituent.

In some embodiments, D has an —OH substituent.

In some embodiments, D has a —CH₃ substituent.

In some embodiments, D has a —CH₂CH₃ substituent.

In some embodiments, D has a —CH₂CH₂CH₃ substituent.

In some embodiments, D has a —CH₂NH₂ substituent.

In some embodiments, D has both —CH₃ and —CH₂NH₂ substituents on the same ring C-atom.

In some embodiments, D has both —NH₂ and —OH substituents.

In some embodiments, D has both —NH₂ and —CH₂CH₃ substituents.

In some embodiments, D has both —NH₂ and —CH₃ substituents.

In some embodiments, D has a —F substituent.

In some embodiments, D has both —F and —CH₃ substituents.

In some embodiments, D has both —F and —CH₂CH₃ substituents.

In some embodiments, D has a —NH—C(O)—CH₂—CN substituent.

In some embodiments, D has a —C(O)—CH₂—CN substituent.

For some compounds, D is represented by formula D1:

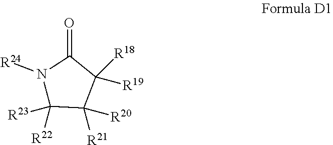

With respect to any relevant structural representation, such as Formula D1, R¹⁸, R¹⁹, R²⁰, R²¹, R²², R²³, and R²⁴ are independently a covalent bond to L, R², H, or any substituent, such as a substituent having a molecular weight of 15-200 Da, 15-150 Da, 15-100 Da, or 15-50 Da. In some embodiments R¹⁸, R¹⁹, R²⁰, R²¹, R²², R²³, R²⁴, and R²⁵ are independently H; F; Cl; Br; I; C₁₋₆H₀₋₁₆N₀₋₃O₀₋₃F₀₋₃; C₀₋₃N₁₋₃O₀₋₃H₀₋₁₀; or C₀₋₃N₀₋₃O₁₋₃H₀₋₁₀. In some embodiments R¹⁸, R¹⁹, R²⁰, R²¹, R²², R²³, and R²⁴ are independently H, C₁₋₄alkyl, C₁₋₃ alkyl-NH₂, or F.

With respect to any relevant structural representation, such as Formula D1, in some embodiments, R¹⁸ is H. In some embodiments, R¹⁸ is F.

With respect to any relevant structural representation, such as Formula D1, in some embodiments, R¹⁹ is H.

With respect to any relevant structural representation, such as Formula D1, in some embodiments, R²⁰ is H. In some embodiments, R²⁰ is methyl. In some embodiments, R²⁰ is ethyl. In some embodiments, R²⁰ is propyl. In some embodiments, R²⁰ is cyclopropyl.

With respect to any relevant structural representation, such as Formula D1, in some embodiments, R²¹ is H.

With respect to any relevant structural representation, such as Formula D1, in some embodiments R²² is a covalent bond to L.

With respect to any relevant structural representation, such as Formula D1, in some embodiments, R²³ is H.

With respect to any relevant structural representation, such as Formula D1, in some embodiments, R²⁴ is H. In some embodiments, R²⁴ is covalent bond to R¹.

In some embodiments, R¹ and L attach to A such that 4 ring atoms of A directly connect R¹ to L. For example, if A is isoquinolinyl, R¹ and L may attach to A as shown below.

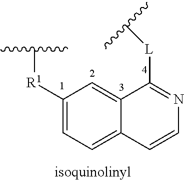

In some embodiments, R is H.

In some embodiments, R is —OCH₃, —OCH₂CH₃, or —OCH(CH₃)₂.

In some embodiments, R is —NHCH₃.

In some embodiments, R is —NH₂.

In some embodiments, L is —O—CH₂—.

In some embodiments, L is —NH—CH₂—.

In some embodiments, L is —CH₂—CH₂—.

In some embodiments, L is

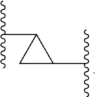 [SMILES: *C1CC1*]

In some embodiments, L is —S(O)₂—CH₂—.

In some embodiments, L is —NH—.

In some embodiments, L is —S—.

In some embodiments, L is —S(O)₂—.

In some embodiments, L is a bond.

In some embodiments, L is a bond, and the N ring atom of the ring D is directly connected to the ring A.

In some embodiments, R² is H.

In some embodiments, R² is —C(O)—.

In some embodiments, R² is a direct covalent bond to R¹.

In some embodiments, there is a covalent bond between R¹ and R², and R¹—R² is —OCH₂CH₂OCH₂—.

In some embodiments, there is a covalent bond between R¹ and R², and R¹—R² is —OCH₂CH₂OCH₂CH₂—.

In some embodiments, there is a covalent bond between R¹ and R², and R¹—R² is —OCH₂CH₂OCH₂CH₂OCH₂—.

In some embodiments, there is a covalent bond between R¹ and R², and R¹—R² is —OCH₂CH₂OCH₂C(O)—.

In some embodiments, R^{A} is H.

In some embodiments, R^{B} is H.

In some embodiments, R^{C} is H.

In some embodiments, R^{D} is H.

Some embodiments include optionally substituted 5-((5-oxopyrrolidin-2-yl)methoxy)-1,3-dihydro-2H-imidazo[4,5-g]isoquinolin-2-one, optionally substituted 7-methoxy-1-((5-oxopyrrolidin-2-yl)methoxy)isoquinolin-6-yl carbamate, optionally substituted 7-(methylamino)-1-((5-oxopyrrolidin-2-yl)methoxy)isoquinoline-6-carboxamide, optionally substituted N-hydroxy-7-methoxy-1-((5-oxopyrrolidin-2-yl)methoxy)isoquinoline-6-carboxamide, optionally substituted 7-methoxy-1-(((5-oxopyrrolidin-2-yl)methyl)amino)isoquinoline-6-carboxamide, optionally substituted 5-((5-oxopyrrolidin-2-yl)methoxy)-1H-pyrrolo[3,4-g]isoquinoline-1,3(2H)-dione, optionally substituted (E)-3-(hydroxyimino)-5-((5-oxopyrrolidin-2-yl)methoxy)-1,3-dihydro-2H-pyrrolo[2,3-g]isoquinolin-2-one, optionally substituted 5-(((6-(2,2-difluoroacetyl)-7-methoxyisoquinolin-1-yl)oxy)methyl)pyrrolidin-2-one, optionally substituted 4⁵-oxo-2,6,9-trioxa-1(1,7)-isoquinolina-4(2,3)-pyrrolidinacyclononaphane-1⁶-carboxamide, optionally substituted 4⁵-oxo-2,6,9-trioxa-1(1,7)-isoquinolina-4(2,1)-pyrrolidinacyclononaphane-1⁶-carboxamide, optionally substituted 4⁵-oxo-2,7,10-trioxa-1(1,7)-isoquinolina-4(2,3)-pyrrolidinacyclodecaphane-1⁶-carboxamide, optionally substituted 4⁵-oxo-2,7,10-trioxa-1(1,7)-isoquinolina-4(2,1)-pyrrolidinacyclodecaphane-1⁶-carboxamide, optionally substituted 1²-oxo-3,5,8-trioxa-1(4,3)-oxazolidina-4(1,7)-isoquinolinacyclononaphane-4⁶-carboxamide, optionally substituted 4²-oxo-2,6,9-trioxa-1(1,7)-isoquinolina-4(4,3)-imidazolidinacyclononaphane-1⁶-carboxamide, optionally substituted (4^{3a}R,4^{6a}R)-5-oxo-4¹,4²,4³,4^{3a},4⁴,4⁵,4⁶,4^{6a}-octahydro-2,7,10-trioxa-1(1,7)-isoquinolina-4(6,2)-cyclopenta[c]pyrrolacyclodecaphane-1⁶-carboxamide, optionally substituted 5-oxo-2,7,10-trioxa-1(1,7)-isoquinolina-4(3,1)-azetidinacyclodecaphane-1⁶-carboxamide, optionally substituted 6-methoxy-1-(2-(5-oxopyrrolidin-2-yl)ethyl)-1H-indole-5-carboxamide, optionally substituted (4^{3a}R,4^{6a}S)-4³,5-dioxo-4¹,4²,4³,4^{3a},4⁴,4⁵,4⁶,4^{6a}-octahydro-2,7,10-trioxa-1(1,7)-isoquinolina-4(1,5)-pyrrolo[3,4-c]pyrrolacyclodecaphane-1⁶-carboxamide, optionally substituted 7-methoxy-1-(2-(5-oxopyrrolidin-2-yl)cyclopropyl)isoquinoline-6-carboxamide, optionally substituted 7-methoxy-9-((5-oxopyrrolidin-2-yl)methoxy)-1H-imidazo[1,2-a]indole-6-carboxamide, optionally substituted 6-methoxy-1-(2-(5-oxopyrrolidin-2-yl)ethyl)-1H-benzo[d]imidazole-5-carboxamide, optionally substituted 7-methoxy-9-((5-oxopyrrolidin-2-yl)methoxy)naphtho[2,3-b]thiophene-6-carboxamide, optionally substituted 7-methoxy-9-((5-oxopyrrolidin-2-yl)methoxy)thiazolo[3,2-a]indole-6-carboxamide, optionally substituted 7-methoxy-1-methyl-9-((5-oxopyrrolidin-2-yl)methoxy)-1H-benzo[f]indole-6-carboxamide, optionally substituted 3-methoxy-5-(((5-oxopyrrolidin-2-yl)methyl)sulfonyl)-2-naphthamide, optionally substituted 5-methoxy-3-((5-oxopyrrolidin-2-yl)methoxy)-1H-indole-6-carboxamide, optionally substituted 3-amino-6-((5-oxopyrrolidin-2-yl)methoxy)benzo[g]quinoxalin-2(1H)-one, optionally substituted 7-methoxy-9-((5-oxopyrrolidin-2-yl)methoxy)naphtho[2,3-b]furan-6-carboxamide, optionally substituted 6-((5-oxopyrrolidin-2-yl)methoxy)-3,4-dihydrobenzo[g]quinoxalin-2(1H)-one, optionally substituted 7-methoxy-9-((5-oxopyrrolidin-2-yl)methoxy)oxazolo[3,2-a]indole-6-carboxamide, optionally substituted 6-((5-oxopyrrolidin-2-yl)methoxy)benzo[g]quinoxalin-2(1H)-one, optionally substituted (S)-7-methoxy-1-((5-oxopyrrolidin-2-yl)methoxy)isoquinoline-6-carbothioamide, optionally substituted (S)—S-(7-methoxy-1-((5-oxopyrrolidin-2-yl)methoxy)isoquinolin-6-yl) carbamothioate, optionally substituted (S)—O-(7-methoxy-1-((5-oxopyrrolidin-2-yl)methoxy)isoquinolin-6-yl) carbamothioate, optionally substituted (S)-1-(7-methoxy-1-((5-oxopyrrolidin-2-yl)methoxy)isoquinolin-6-yl)thiourea, optionally substituted (S)-7-methoxy-1-((5-oxopyrrolidin-2-yl)methoxy)isoquinoline-6-carbothioic S-acid, optionally substituted (S,E)-1-(7-methoxy-1-((5-oxopyrrolidin-2-yl)methoxy)isoquinolin-6-yl)-2-methylguanidine, optionally substituted S-methyl (S)-(7-methoxy-1-((5-oxopyrrolidin-2-yl)methoxy)isoquinolin-6-yl)carbamothioate, optionally substituted methyl (S)-(7-methoxy-1-((5-oxopyrrolidin-2-yl)methoxy)isoquinolin-6-yl)carbamate, optionally substituted (S)-1-(3-aminopiperidin-1-yl)-7-methoxyisoquinoline-6-carboxamide, optionally substituted (S)-1-(3-hydroxypiperidin-1-yl)-7-methoxyisoquinoline-6-carboxamide, optionally substituted 1-(4-aminopiperidin-1-yl)-7-methoxyisoquinoline-6-carboxamide, optionally substituted 1-((1S,3S)-3-aminocyclopentyl)-6-methoxy-1H-indole-5-carboxamide, optionally substituted (S)-7-methoxy-1-(((5-oxopyrrolidin-2-yl)methyl)amino)-4-(prop-1-yn-1-yl)isoquinoline-6-carboxamide, optionally substituted (4²,4³R)-4³-ethyl-4⁵-oxo-2,6,9-trioxa-1(1,7)-isoquinolina-4(2,1)-pyrrolidinacyclononaphane-1⁶-carboxamide, optionally substituted (4²,4³R)-4³-methyl-4⁵-oxo-2,6,9-trioxa-1(1,7)-isoquinolina-4(2,1)-pyrrolidinacyclononaphane-1⁶-carboxamide, optionally substituted (4²S,4³R)-4⁵-oxo-4³-propyl-2,6,9-trioxa-1(1,7)-isoquinolina-4(2,1)-pyrrolidinacyclononaphane-1⁶-carboxamide, optionally substituted 1-(4-(aminomethyl)-4-methylpiperidin-1-yl)-7-methoxyisoquinoline-6-carboxamide, optionally substituted 1-((3R,4S)-3-amino-4-ethylpyrrolidin-1-yl)-7-methoxyisoquinoline-6-carboxamide, optionally substituted 1-((3R,5S)-3-amino-5-hydroxypiperidin-1-yl)-7-methoxyisoquinoline-6-carboxamide, optionally substituted (S)-1-(3-aminopiperazin-1-yl)-7-methoxyisoquinoline-6-carboxamide, optionally substituted (4²S,4³R)-4³-methyl-4⁵-oxo-1⁴-(prop-1-yn-1-yl)-2,6,9-trioxa-1(1,7)-isoquinolina-4(2,1)-pyrrolidinacyclononaphane-1⁶-carboxamide, optionally substituted 41H-2,6,9-trioxa-1(1,7)-isoquinolina-4(5,1)-triazolacyclononaphane-1⁶-carboxamide, optionally substituted (S)-2-amino-7-methoxy-5-(((5-oxopyrrolidin-2-yl)methyl)amino)-3H-imidazo[4,5-c]isoquinoline-8-carboxamide, optionally substituted (S)-2-amino-7-methoxy-5-(((5-oxopyrrolidin-2-yl)methyl)amino)thiazolo[5,4-c]isoquinoline-8-carboxamide, optionally substituted (S)-2-amino-7-methoxy-5-(((5-oxopyrrolidin-2-yl)methyl)amino)oxazolo[5,4-c]isoquinoline-8-carboxamide, optionally substituted 1-((((2S,3R)-3-ethyl-5-oxopyrrolidin-2-yl)methyl)amino)-7-methoxyisoquinoline-6-carboxamide, optionally substituted (4²S,4³S,4⁴S)-4⁴-fluoro-4³-methyl-4⁵-oxo-2,6,9-trioxa-1(1,7)-isoquinolina-4(2,1)-pyrrolidinacyclononaphane-1⁶-carboxamide, optionally substituted (4²S,4³,4⁴S)-4³-ethyl-4⁴-fluoro-4⁵-oxo-2,6,9-trioxa-1(1,7)-isoquinolina-4(2,1)-pyrrolidinacyclononaphane-1⁶-carboxamide, optionally substituted 1-((3S,4R)-4-amino-3-methyl-2-oxa-8-azaspiro[4.5]decan-8-yl)-7-methoxyisoquinoline-6-carboxamide, optionally substituted (S)-1-(3-(2-cyanoacetamido)piperidin-1-yl)-7-methoxyisoquinoline-6-carboxamide, optionally substituted (R)-7-methoxy-1-(piperidin-3-ylamino)isoquinoline-6-carboxamide, optionally substituted (R)-1-((1-(2-cyanoacetyl)piperidin-3-yl)amino)-7-methoxyisoquinoline-6-carboxamide, optionally substituted (R)-7-methoxy-1-(piperidin-3-ylthio)isoquinoline-6-carboxamide, optionally substituted (R)-1-((1-(2-cyanoacetyl)piperidin-3-yl)thio)-7-methoxyisoquinoline-6-carboxamide, optionally substituted (R)-7-methoxy-1-(piperidin-3-ylsulfonyl)isoquinoline-6-carboxamide, optionally substituted (R)-1-((1-(2-cyanoacetyl)piperidin-3-yl)sulfonyl)-7-methoxyisoquinoline-6-carboxamide, optionally substituted (R)-1-((1-(2-cyanoacetyl)pyrrolidin-3-yl)amino)-7-methoxyisoquinoline-6-carboxamide, optionally substituted (S)-1-(3-aminopiperidin-1-yl)-7-methoxy-4-(prop-1-yn-1-yl)isoquinoline-6-carboxamide, optionally substituted 1-(4-aminopiperidin-1-yl)-7-methoxy-4-(prop-1-yn-1-yl)isoquinoline-6-carboxamide, optionally substituted (S)-1-(3-aminopiperidin-1-yl)-4-bromo-7-methoxyisoquinoline-6-carboxamide, optionally substituted (S)-1-(3-aminopiperidin-1-yl)-4-cyano-7-methoxyisoquinoline-6-carboxamide, optionally substituted (R)-7-methoxy-1-(pyrrolidin-3-ylamino)isoquinoline-6-carboxamide, optionally substituted 7-methoxy-1-((((2S,3R)-3-methyl-5-oxopyrrolidin-2-yl)methyl)amino)-4-(prop-1-yn-1-yl)isoquinoline-6-carboxamide, optionally substituted 1-((((2S,3R)-3-ethyl-5-oxopyrrolidin-2-yl)methyl)amino)-7-methoxy-4-(prop-1-yn-1-yl)isoquinoline-6-carboxamide, optionally substituted 1-((3S,5S)-3-amino-5-ethylpiperidin-1-yl)-7-methoxy-4-(prop-1-yn-1-yl)isoquinoline-6-carboxamide, optionally substituted 1-((3S,5S)-3-amino-5-ethylpiperidin-1-yl)-7-methoxyisoquinoline-6-carboxamide, optionally substituted 1-((3S,4R)-3-amino-4-ethylpiperidin-1-yl)-7-methoxyisoquinoline-6-carboxamide, optionally substituted 1-((3S,4R)-3-amino-4-ethylpiperidin-1-yl)-7-methoxy-4-(prop-1-yn-1-yl)isoquinoline-6-carboxamide, optionally substituted 1-((((3S,4S)-3-ethyl-4-fluoro-5-oxopyrrolidin-2-yl)methyl)amino)-7-methoxyisoquinoline-6-carboxamide, optionally substituted 1-((((3S,4S)-4-fluoro-3-methyl-5-oxopyrrolidin-2-yl)methyl)amino)-7-methoxyisoquinoline-6-carboxamide, optionally substituted 1-((((3S,4S)-3-ethyl-4-fluoro-5-oxopyrrolidin-2-yl)methyl)amino)-7-methoxy-4-(prop-1-yn-1-yl)isoquinoline-6-carboxamide, or optionally substituted 1-((((3S,4S)-4-fluoro-3-methyl-5-oxopyrrolidin-2-yl)methyl)amino)-7-methoxy-4-(prop-1-yn-1-yl)isoquinoline-6-carboxamide.

Some embodiments include any compound or any structure described herein, wherein any compound or any structure described herein may be optionally substituted.

Some embodiments include one of the compounds below in Table I, wherein any one of the below compounds may be optionally substituted.

TABLE I

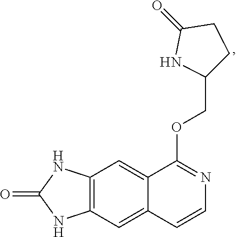 [SMILES: O=C1CCC(COc2nccc3cc4[nH]c(=O)[nH]c4cc23)N1]

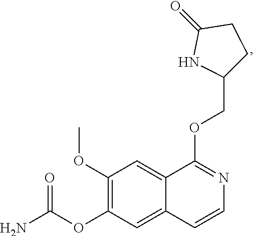 [SMILES: COc1cc2c(OCC3CCC(=O)N3)nccc2cc1OC(N)=O]

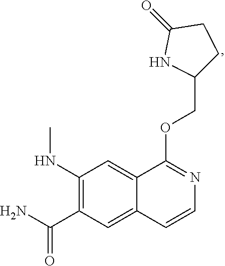 [SMILES: CNc1cc2c(OCC3CCC(=O)N3)nccc2cc1C(N)=O]

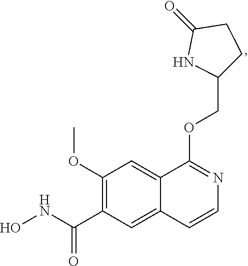 [SMILES: COc1cc2c(OCC3CCC(=O)N3)nccc2cc1C(=O)NO]

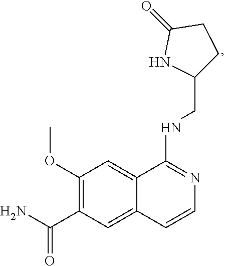 [SMILES: COc1cc2c(NCC3CCC(=O)N3)nccc2cc1C(N)=O]

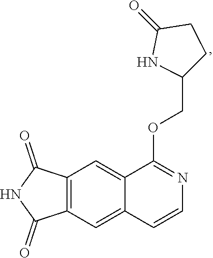 [SMILES: O=C1CCC(COc2nccc3cc4c(cc23)C(=O)NC4=O)N1]

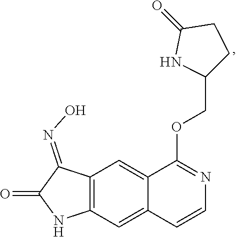 [SMILES: O=C1CCC(COc2nccc3cc4c(cc23)/C(=N\O)C(=O)N4)N1]

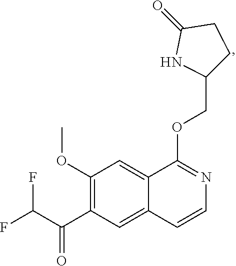 [SMILES: COc1cc2c(OCC3CCC(=O)N3)nccc2cc1C(=O)C(F)F]

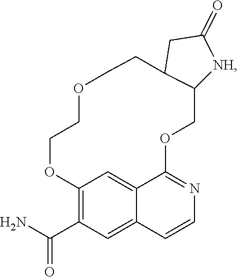 [SMILES: NC(=O)c1cc2ccnc3c2cc1OCCOCCC1CC(=O)NC1CO3]

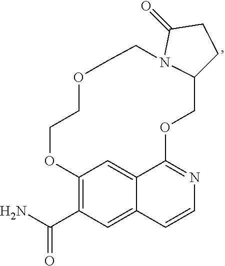 [SMILES: NC(=O)c1cc2ccnc3c2cc1OCCOCCN1C(=O)CCC1CO3]

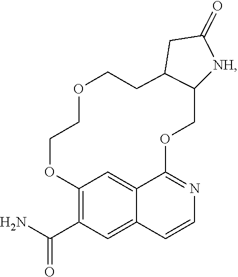 [SMILES: NC(=O)c1cc2ccnc3c2cc1OCCOCCC1CC(=O)NC1CO3]

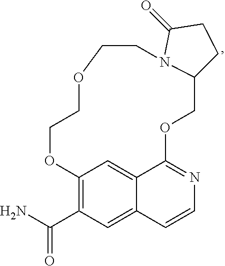 [SMILES: NC(=O)c1cc2ccnc3c2cc1OCCOCCN1C(=O)CCC1CO3]

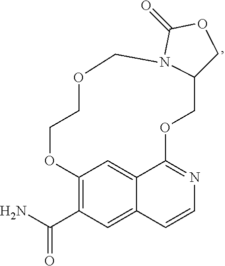 [SMILES: NC(=O)c1cc2ccnc3c2cc1OCCOCN1C(=O)OCC1CO3]

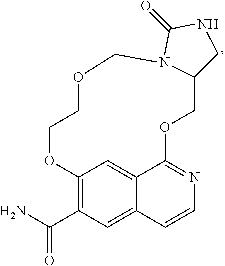 [SMILES: NC(=O)c1cc2ccnc3c2cc1OCCOCN1C(=O)NCC1CO3]

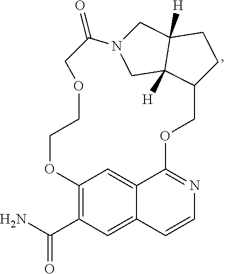 [SMILES: NC(=O)c1cc2ccnc3c2cc1OCCOCC(=O)N1C[C@@H]2CCC(CO3)[C@@H]2C1]

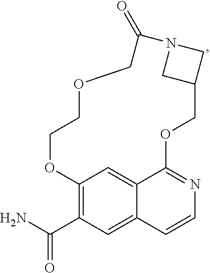 [SMILES: NC(=O)c1cc2ccnc3c2cc1OCCOCC(=O)N1CC(CO3)C1]

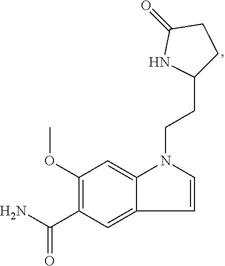 [SMILES: COc1cc2c(ccn2CCC2CCC(=O)N2)cc1C(N)=O]

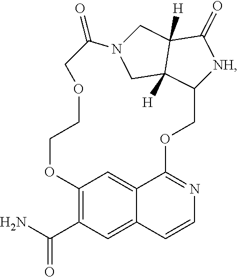 [SMILES: NC(=O)c1cc2ccnc3c2cc1OCCOCC(=O)N1C[C@@H]2C(=O)NC(CO3)[C@@H]2C1]

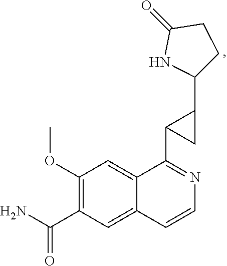 [SMILES: COc1cc2c(C3CC3C3CCC(=O)N3)nccc2cc1C(N)=O]

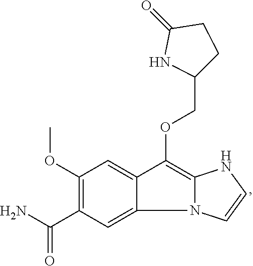 [SMILES: COc1cc2c(OCC3CCC(=O)N3)c3[nH]ccn3c2cc1C(N)=O]

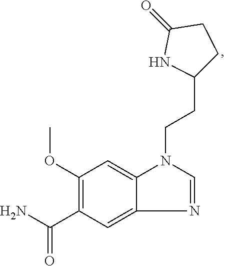 [SMILES: COc1cc2c(cc1C(N)=O)ncn2CCC1CCC(=O)N1]

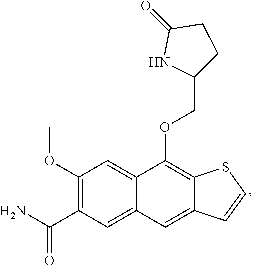 [SMILES: COc1cc2c(OCC3CCC(=O)N3)c3sccc3cc2cc1C(N)=O]

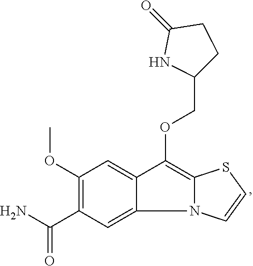 [SMILES: COc1cc2c(OCC3CCC(=O)N3)c3sccn3c2cc1C(N)=O]

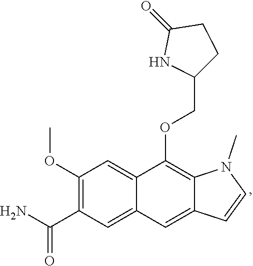 [SMILES: COc1cc2c(OCC3CCC(=O)N3)c3c(ccn3C)cc2cc1C(N)=O]

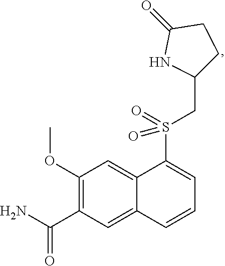 [SMILES: COc1cc2c(S(=O)(=O)CC3CCC(=O)N3)cccc2cc1C(N)=O]

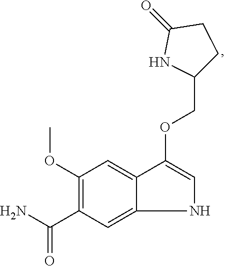 [SMILES: COc1cc2c(OCC3CCC(=O)N3)c[nH]c2cc1C(N)=O]

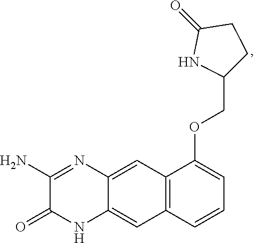 [SMILES: Nc1nc2cc3c(OCC4CCC(=O)N4)cccc3cc2[nH]c1=O]

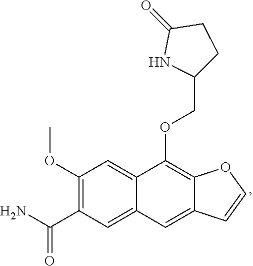 [SMILES: COc1cc2c(OCC3CCC(=O)N3)c3occc3cc2cc1C(N)=O]

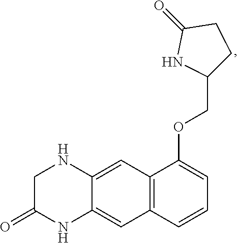 [SMILES: O=C1CNc2cc3c(OCC4CCC(=O)N4)cccc3cc2N1]

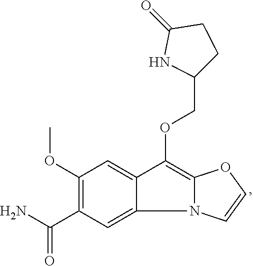 [SMILES: COc1cc2c(OCC3CCC(=O)N3)c3occn3c2cc1C(N)=O]

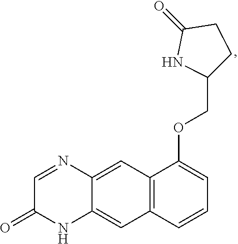 [SMILES: O=C1CCC(COc2cccc3cc4[nH]c(=O)cnc4cc23)N1]

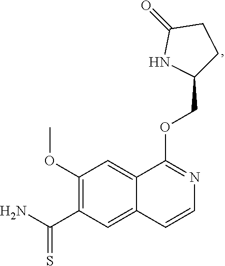 [SMILES: COc1cc2c(OC[C@@H]3CCC(=O)N3)nccc2cc1C(N)=S]

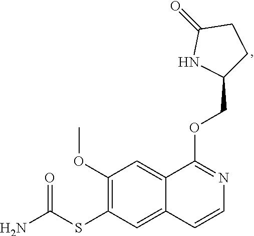 [SMILES: COc1cc2c(OC[C@@H]3CCC(=O)N3)nccc2cc1SC(N)=O]

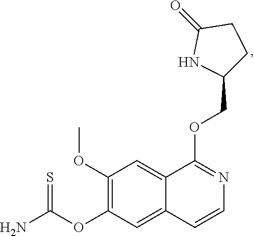 [SMILES: COc1cc2c(OC[C@@H]3CCC(=O)N3)nccc2cc1OC(N)=S]

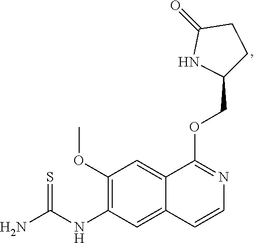 [SMILES: COc1cc2c(OC[C@@H]3CCC(=O)N3)nccc2cc1NC(N)=S]

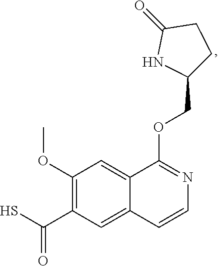 [SMILES: COc1cc2c(OC[C@@H]3CCC(=O)N3)nccc2cc1C(=O)S]

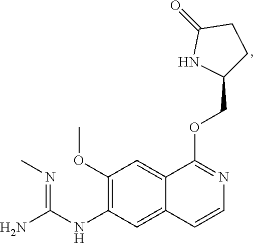 [SMILES: C/N=C(\N)Nc1cc2ccnc(OC[C@@H]3CCC(=O)N3)c2cc1OC]

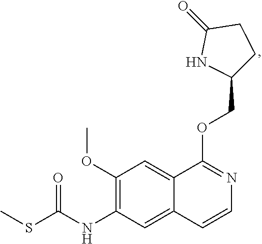 [SMILES: COc1cc2c(OC[C@@H]3CCC(=O)N3)nccc2cc1NC(=O)SC]

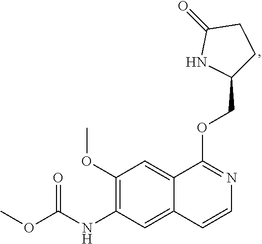 [SMILES: COC(=O)Nc1cc2ccnc(OC[C@@H]3CCC(=O)N3)c2cc1OC]

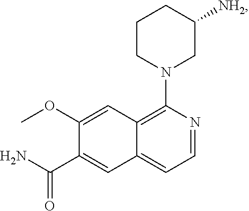 [SMILES: COc1cc2c(N3CCC[C@H](N)C3)nccc2cc1C(N)=O]

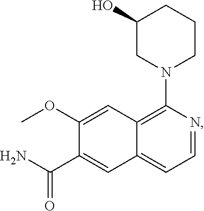 [SMILES: COc1cc2c(N3CCC[C@H](O)C3)nccc2cc1C(N)=O]

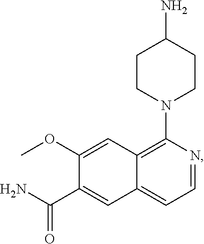 [SMILES: COc1cc2c(N3CCC(N)CC3)nccc2cc1C(N)=O]

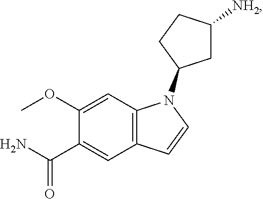 [SMILES: COc1cc2c(ccn2[C@H]2CC[C@H](N)C2)cc1C(N)=O]

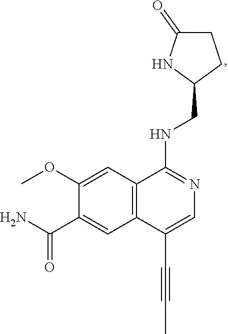 [SMILES: CC#Cc1cnc(NC[C@@H]2CCC(=O)N2)c2cc(OC)c(C(N)=O)cc12]

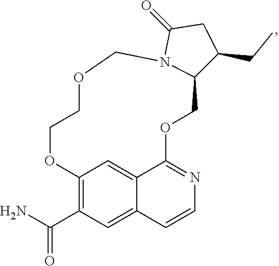 [SMILES: CC[C@@H]1CC(=O)N2COCCOc3cc4c(nccc4cc3C(N)=O)OC[C@H]12]

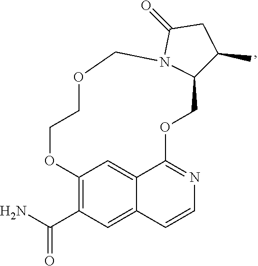 [SMILES: C[C@@H]1CC(=O)N2COCCOc3cc4c(nccc4cc3C(N)=O)OC[C@H]12]

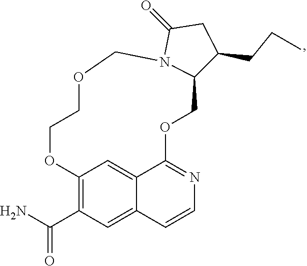 [SMILES: CCC[C@@H]1CC(=O)N2COCCOc3cc4c(nccc4cc3C(N)=O)OC[C@H]12]

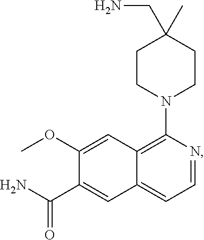 [SMILES: COc1cc2c(N3CCC(C)(CN)CC3)nccc2cc1C(N)=O]

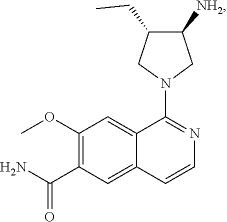 [SMILES: CC[C@H]1CN(c2nccc3cc(C(N)=O)c(OC)cc23)C[C@@H]1N]

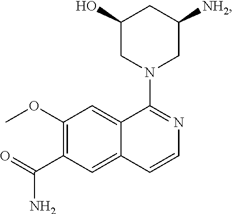 [SMILES: COc1cc2c(N3C[C@H](N)C[C@H](O)C3)nccc2cc1C(N)=O]

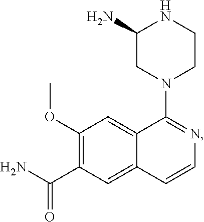 [SMILES: COc1cc2c(N3CCN[C@H](N)C3)nccc2cc1C(N)=O]

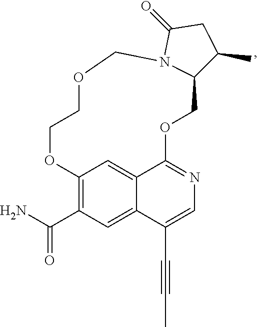 [SMILES: CC#Cc1cnc2c3cc(c(C(N)=O)cc13)OCCOCN1C(=O)C[C@@H](C)[C@H]1CO2]

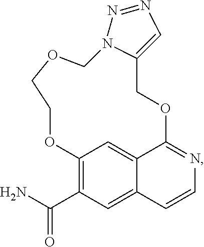 [SMILES: NC(=O)c1cc2ccnc3c2cc1OCCOCn1nncc1CO3]

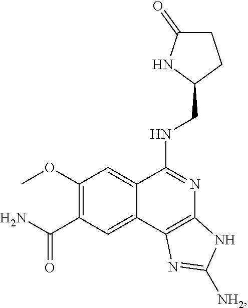 [SMILES: COc1cc2c(NC[C@@H]3CCC(=O)N3)nc3[nH]c(N)nc3c2cc1C(N)=O]

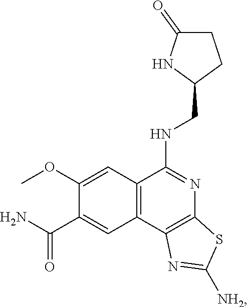 [SMILES: COc1cc2c(NC[C@@H]3CCC(=O)N3)nc3sc(N)nc3c2cc1C(N)=O]

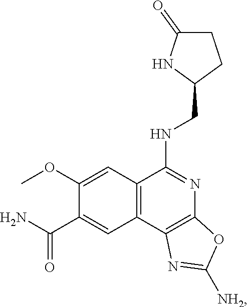 [SMILES: COc1cc2c(NC[C@@H]3CCC(=O)N3)nc3oc(N)nc3c2cc1C(N)=O]

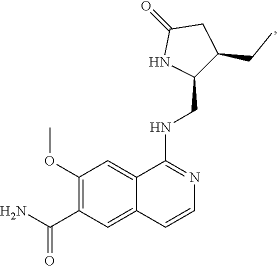 [SMILES: CC[C@@H]1CC(=O)N[C@@H]1CNc1nccc2cc(C(N)=O)c(OC)cc12]

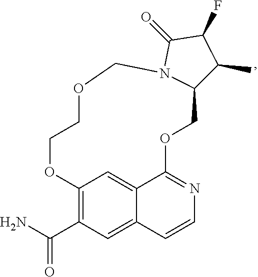 [SMILES: C[C@@H]1[C@H](F)C(=O)N2COCCOc3cc4c(nccc4cc3C(N)=O)OC[C@H]12]

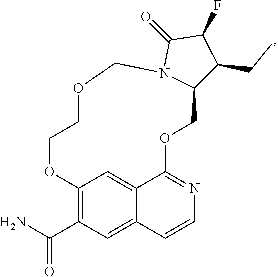 [SMILES: CC[C@@H]1[C@H](F)C(=O)N2COCCOc3cc4c(nccc4cc3C(N)=O)OC[C@H]12]

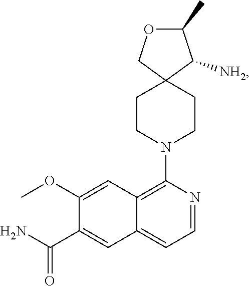 [SMILES: COc1cc2c(N3CCC4(CC3)CO[C@@H](C)[C@@H]4N)nccc2cc1C(N)=O]

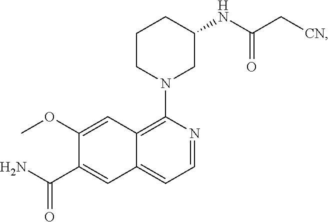 [SMILES: COc1cc2c(N3CCC[C@H](NC(=O)CC#N)C3)nccc2cc1C(N)=O]

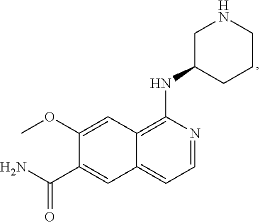 [SMILES: COc1cc2c(N[C@@H]3CCCNC3)nccc2cc1C(N)=O]

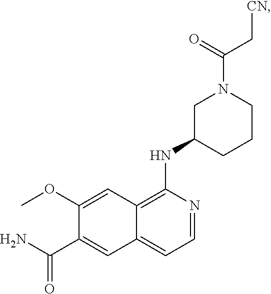 [SMILES: COc1cc2c(N[C@@H]3CCCN(C(=O)CC#N)C3)nccc2cc1C(N)=O]

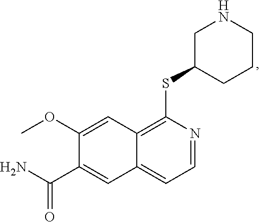 [SMILES: COc1cc2c(S[C@@H]3CCCNC3)nccc2cc1C(N)=O]

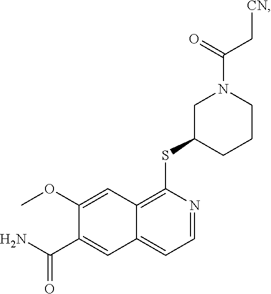 [SMILES: COc1cc2c(S[C@@H]3CCCN(C(=O)CC#N)C3)nccc2cc1C(N)=O]

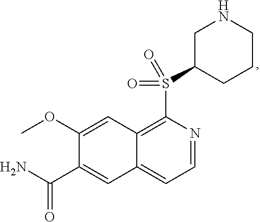 [SMILES: COc1cc2c(S(=O)(=O)[C@@H]3CCCNC3)nccc2cc1C(N)=O]

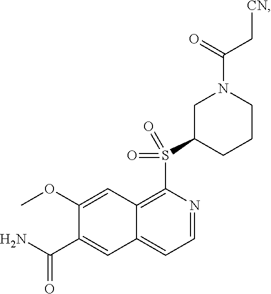 [SMILES: COc1cc2c(S(=O)(=O)[C@@H]3CCCN(C(=O)CC#N)C3)nccc2cc1C(N)=O]

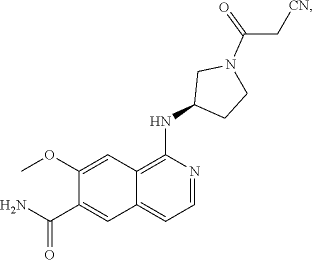 [SMILES: COc1cc2c(N[C@@H]3CCN(C(=O)CC#N)C3)nccc2cc1C(N)=O]

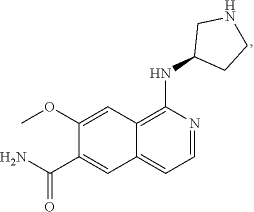 [SMILES: COc1cc2c(N[C@@H]3CCNC3)nccc2cc1C(N)=O]

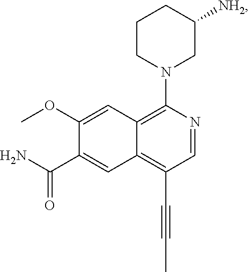 [SMILES: CC#Cc1cnc(N2CCC[C@H](N)C2)c2cc(OC)c(C(N)=O)cc12]

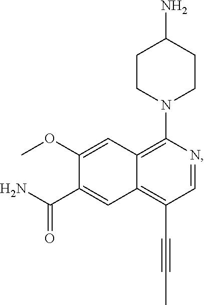 [SMILES: CC#Cc1cnc(N2CCC(N)CC2)c2cc(OC)c(C(N)=O)cc12]

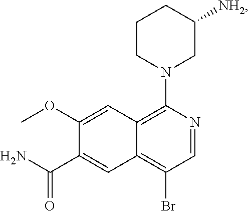 [SMILES: COc1cc2c(N3CCC[C@H](N)C3)ncc(Br)c2cc1C(N)=O]

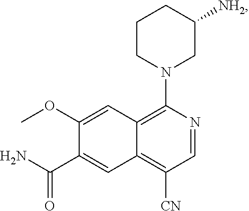 [SMILES: COc1cc2c(N3CCC[C@H](N)C3)ncc(C#N)c2cc1C(N)=O]

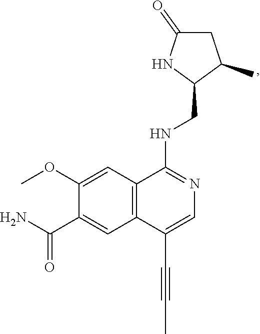 [SMILES: CC#Cc1cnc(NC[C@H]2NC(=O)C[C@H]2C)c2cc(OC)c(C(N)=O)cc12]

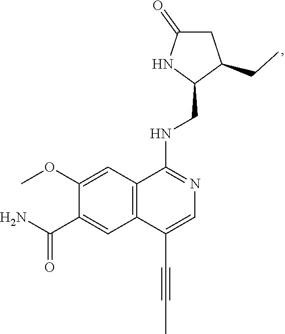 [SMILES: CC#Cc1cnc(NC[C@H]2NC(=O)C[C@H]2CC)c2cc(OC)c(C(N)=O)cc12]

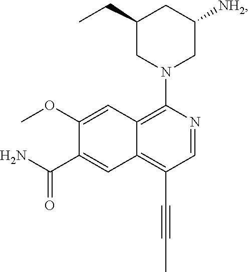 [SMILES: CC#Cc1cnc(N2C[C@@H](N)C[C@H](CC)C2)c2cc(OC)c(C(N)=O)cc12]

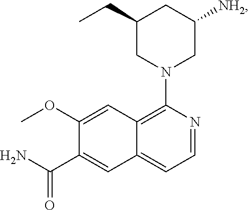 [SMILES: CC[C@H]1C[C@H](N)CN(c2nccc3cc(C(N)=O)c(OC)cc23)C1]

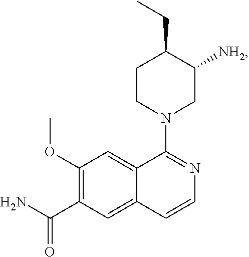 [SMILES: CC[C@@H]1CCN(c2nccc3cc(C(N)=O)c(OC)cc23)C[C@H]1N]

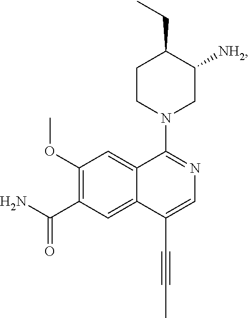 [SMILES: CC#Cc1cnc(N2CC[C@@H](CC)[C@H](N)C2)c2cc(OC)c(C(N)=O)cc12]

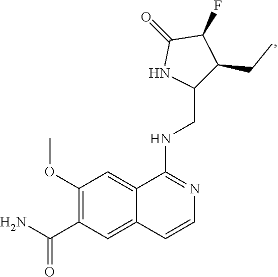 [SMILES: CC[C@H]1C(CNc2nccc3cc(C(N)=O)c(OC)cc23)NC(=O)[C@H]1F]

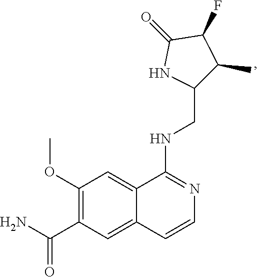 [SMILES: COc1cc2c(NCC3NC(=O)[C@@H](F)[C@H]3C)nccc2cc1C(N)=O]

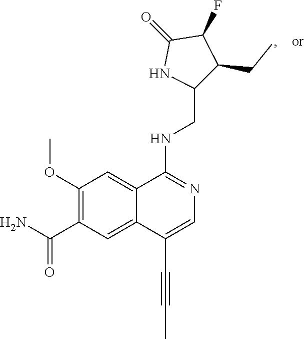 [SMILES: CC#Cc1cnc(NCC2NC(=O)[C@@H](F)[C@H]2CC)c2cc(OC)c(C(N)=O)cc12]

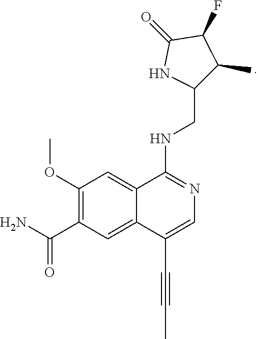 [SMILES: CC#Cc1cnc(NCC2NC(=O)[C@@H](F)[C@H]2C)c2cc(OC)c(C(N)=O)cc12]

A pharmaceutical composition comprising a compound of Formula 1 may be adapted for oral, or parental, such as intravenous, intramuscular, topical, intraperitoneal, nasal, buccal, sublingual, or subcutaneous administration, or for administration via respiratory tract in the form of, for example, an aerosol or an air-suspended fine powder. The dosage of a compound of Formula 1 may vary depending on the route of administration, body weight, age, the type and condition of the disease being treated. A pharmaceutical composition provided herein may optionally comprise two or more compounds of the Formula 1 without an additional therapeutic agent, or may comprise an additional therapeutic agent (i.e., a therapeutic agent other than a compound provided herein). For example, the subject compounds can be used in combination with at least one other therapeutic agent. Therapeutic agents include, but are not limited to antibiotics, antiemetic agents, antidepressants, and antifungal agents, anti-inflammatory agents, antiviral agents, and anticancer agents that are known in the art. The pharmaceutical composition may be used for the treatment of cancer, autoimmune, inflammatory diseases, and autoinflammatory conditions related to IRAK overexpression in patients. The term “patient” herein means a mammal (e.g., a human or an animal). In some embodiments, the patient has cancer.

The pharmaceutical composition described herein can be prepared by combining a compound of Formula 1 with at least one pharmaceutical acceptable inert ingredient, such as a carrier, excipient, filler, lubricant, flavoring agent, buffer, etc., selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

Some embodiments include a method of treating a disease or disorder associated with IRAK4 overexpression comprising administering a therapeutically effective amount of a compound of Formula 1, or a pharmaceutical composition comprising a compound of Formula 1 to a patient in need thereof. The term a “therapeutically effective amount” herein refers to an amount of a subject compound, or a pharmaceutical composition containing a subject compound, sufficient to be effective in inhibiting IRAK4 enzyme and thus providing a benefit in the treatment of cancer, autoimmune, inflammatory diseases, and autoinflammatory conditions related to IRAK overexpression, to delay or minimize symptoms associated with cancer, autoimmune, inflammatory diseases, and autoinflammatory conditions related to IRAK4 overexpression, or to ameliorate a disease or infection or cause thereof. The term “treatment” refers to causing a therapeutically beneficial effect, such as ameliorating existing symptoms, ameliorating the underlying causes of symptoms, postponing, preventing the further development of a disorder, or reducing the severity of symptoms that are otherwise expected to develop without treatment.

### Experimental Section:

### General Synthetic Methods:

The compounds of the present invention, or their pharmaceutically acceptable salts, can be synthesized using the methods described below in schemes 1-7. It will be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that optimum reaction conditions may vary with the particular reactants or solvents used, but such conditions can be determined by the person skilled in the art, using routine optimization procedures. Additionally, one skilled in the art will recognize that in many cases, these compounds will be mixtures of stereoisomers that may be separated at various stages of the synthetic schemes using conventional techniques, such as, but limited to, crystallization, normal-phase chromatography, reversed phase chromatography and chiral chromatography, to afford single enantiomers. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3rd Edition 1999. The schemes 1-5 are representative of methods useful in synthesizing the compounds of the present invention. They are not to constrain the scope of the invention in any way.

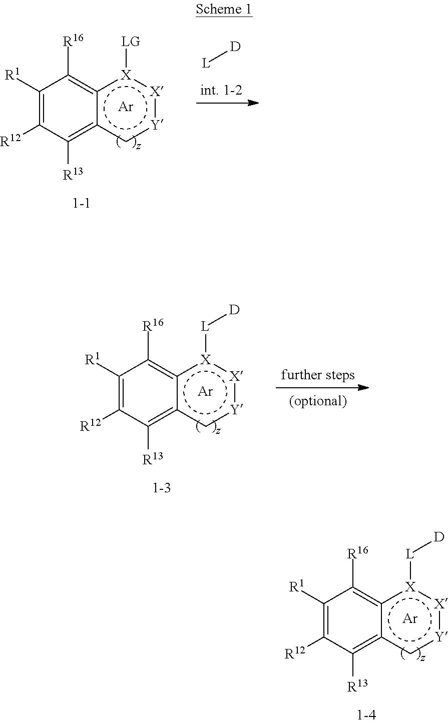

Wherein X, X′ and Y′ are each independently C or N; z=0 or 1; LG is a leaving group such as Cl.

Scheme 1 illustrates a method for preparing compounds of Formula I. Compound 1-1 with a displaceable leaving group (such as chloro) in the presence of a suitable base (such Cs₂CO₃, NaH, ^{t}BuOK, KHMDS) is treated with compound 1-2 to provide a product of formula 1-3. If desired, further transformations may be performed to provide a product of formula 1-4. For example, the compound of Formula 1-3 wherein R¹²═CN may be subjected to a nitrile hydrolysis reaction to provide a compound of Formula 1-4 in which R¹²═CONH₂. In other cases, the compound of Formula 1-3 wherein R¹²═CO₂H may be further treated with reagents, such as NH₂OH, MeSO₂NH₂, NH₂CN, NaSH, to derivatize functional groups. Alternatively compounds of Formula 1-3 wherein R¹² is halogen, such as Br or I, for example, may be subjected to transformations in a variety of ways known to those skilled in the art, for example, such as treatment with TMSN₃ with copper catalyst, or Pd-catalyzed Buchwald-Hartwig amination, to introduce amine group, which may be further derivatized to furnish a product of Formula 1-4, wherein R¹² is a functionalized amine or thiol such as acetyl amine, carbamoyl amine, methoxy formyl amine, amidine, methylthio formyl amine, carbamothioate, and thiourea.

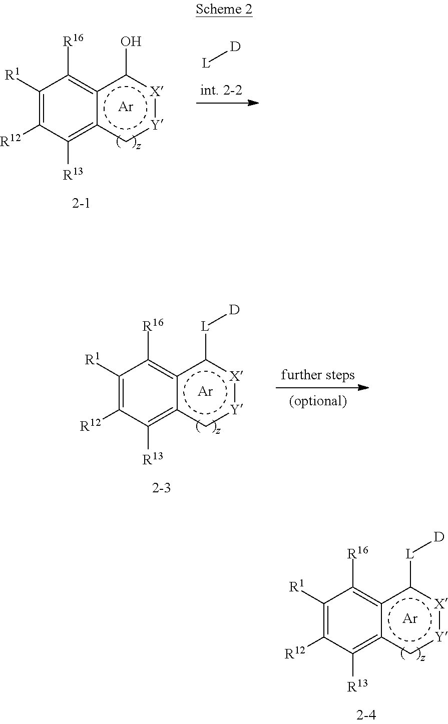

Scheme 2 illustrates another method for the preparation of compounds of Formula I. This method provides for the alkylation of a compound of Formula 2-1 with a compound of Formula 2-2 using methods in a variety of ways known to those skilled in the art, such as Mitsunobu reaction for example, to furnish a product of Formula 2-3. Alternatively, the alkylation of a compound of Formula 2-1 may be carried out in the presence of a base using a compound of Formula 2-2 with a leaving group, such as TsO. Further transformations of R¹² in the compound of Formula 2-3 may be performed to provide a product of formula 2-4, as described in Scheme 1.

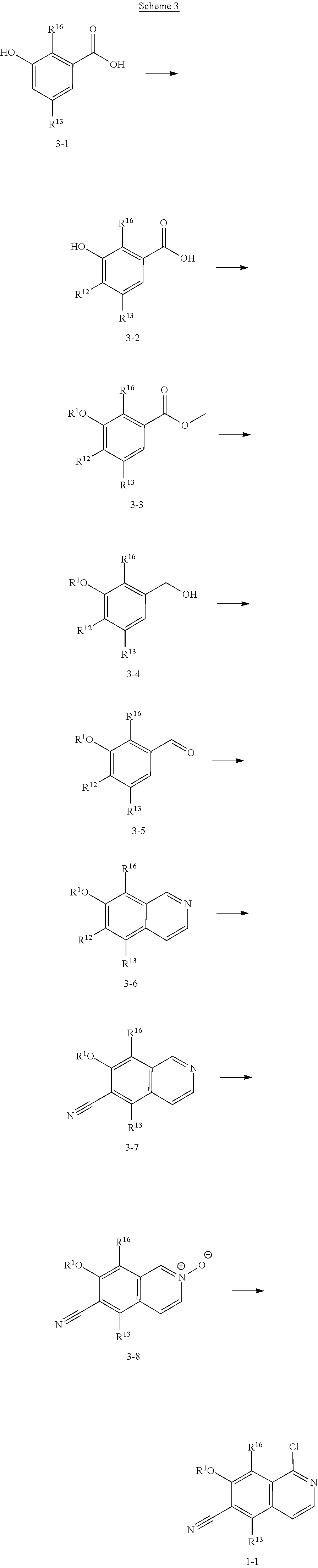

Scheme 3 illustrates a method for preparing compounds of Formula 1-1, as illustrated above. Subsequently a compound of Formula 3-1 is halogenated by reacting with halogen, for example I₂, and then alkylated with an alkylating reagent, for example MeI, to provide the ester of Formula 3-3. The resulting ester is then reduced to a compound of Formula 3-4 by reacting with a suitable reducing agent, such as NaBH₄ or LiBH₄, in a solvent such as THF. Using methods known to those skilled in the art, the alcohol of Formula 3-4 is oxidized to an aldehyde of Formula 3-5. The isoquinoline ring is formed subsequently by reacting with an aminoacetaldehyde acetal followed by the treatment with boron trifluoride etherate, as described in Synthetic Communications 1999, 29 (9), p. 1617. The resulting isoquinoline of Formula 3-6 is cyanated, to afford a nitrile of Formula 3-7. Oxidation with a suitable oxidizing agent, for example H₂O₂ or m-CPBA, yields an isoquinoline N-oxide of Formula 3-8. Halogenation by methods known to those skilled in the art, frequently by with POCl₃, furnishes the intermediate of Formula 1-1 (LG=Cl).

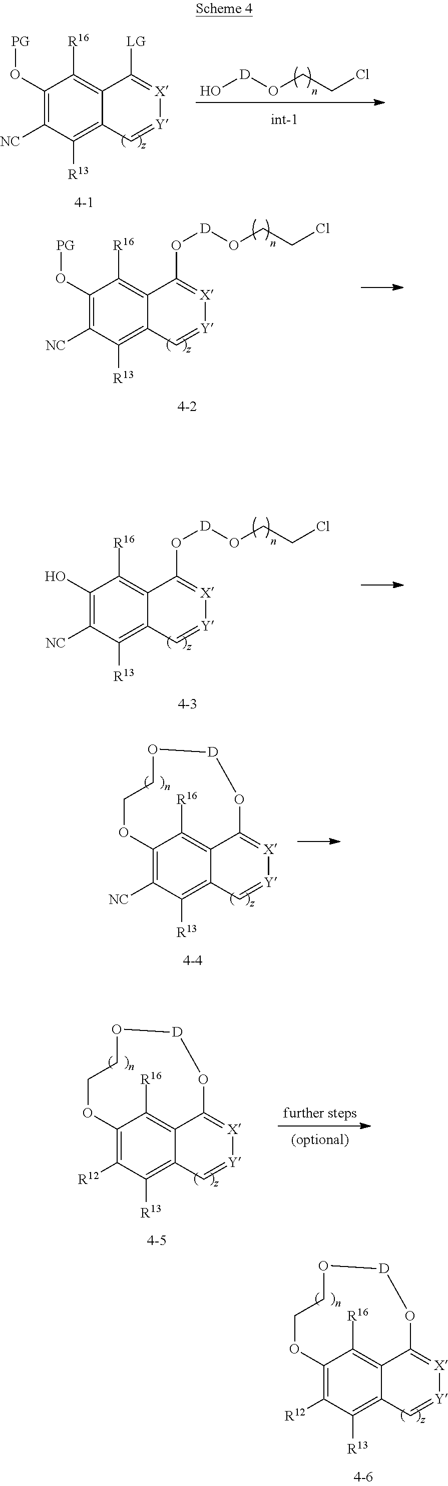

Wherein PG is a protecting group such as Bn.

Scheme 4 illustrates a method for preparing compounds of Formula I suited to these instances in which a macrocyclic ring is formed. Compound of Formula 4-1 with a displaceable leaving group (such as chloro, for example) in the presence of a suitable base (such Cs₂CO₃, NaH, ^{t}BuOK, KHMDS) is treated with int-1 to provide a product of formula 4-2. Further transformations such as de-protection and base-catalyzed displacement using methods known to those skilled in the art are performed to furnish a product of Formula 4-4, wherein CN may be subjected to a nitrile hydrolysis reaction to provide a compound of Formula 4-5 in which R¹²═CONH₂. In other cases, the compound of Formula 4-5 wherein R¹²═CO₂H may be further treated with reagents, such as NH₂OH, MeSO₂NH₂, NH₂CN, to derivatize functional groups.

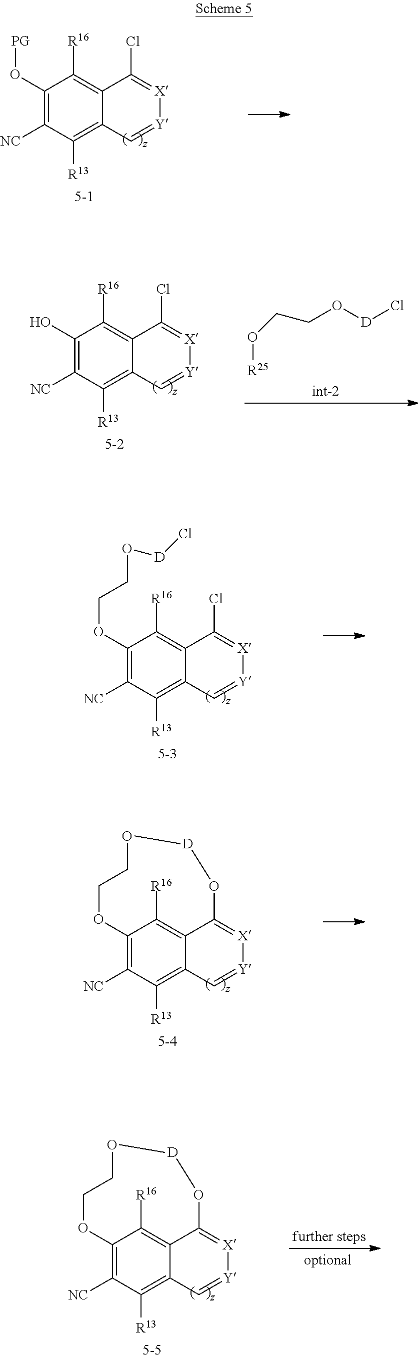

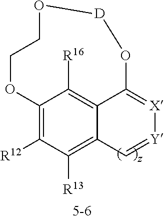

Alternatively, as illustrated in Scheme 5, the order of reactions can be modified to change the overall synthesis to allow for variations at different positions of the molecule at different stages of the preparation. For example, in Scheme 5, compound of Formula 5-1 is de-protected first, and then reacted with int-2 using methods known to those skilled in the art, such as Mitsunobo reaction (R²⁵=H) or displacement reaction (R²⁵=Ts or Ms) for example, to provide compound of Formula 5-2. Cyclization of compound of Formula 5-3, followed by further transformations can afford the compounds of Formula 5-5. In some cases, the compound of Formula 5-5 may be further derivatized to compound of Formula 5-6, as described in Scheme 1.

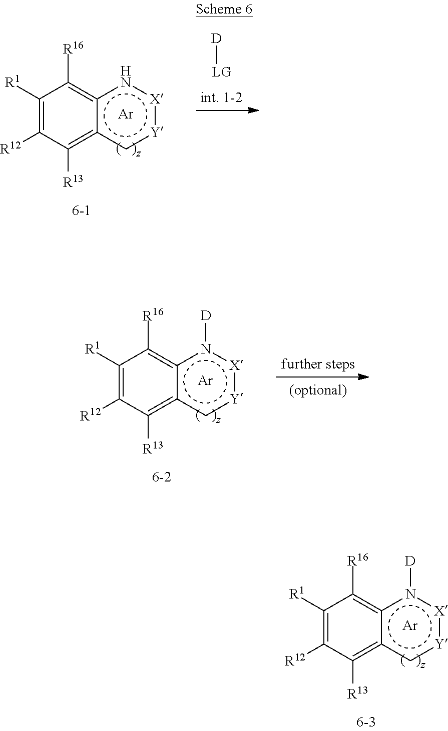

Scheme 6 illustrates a method for preparing compounds of Formula I. Compound of formula 6-1 reacted with compound of formula 1-2 on which there is a leaving group (such as chloro, for example) in the presence of a suitable base (such as Cs₂CO₃, NaH, ^{t}BuOK, KHMDS, KOH) to provide a product of formula 6-2. If desired, further transformations may be performed to provide a product of formula 6-3. For example, the compound of Formula 6-2 wherein R¹²=CN may be subjected to a nitrile hydrolysis reaction to provide a compound of Formula 6-3 in which R¹²=CON H₂.

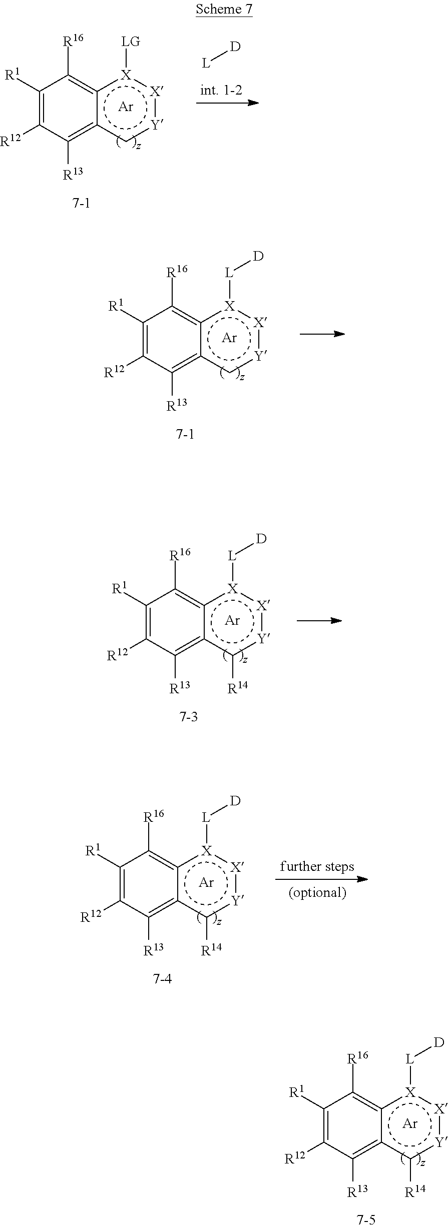

Scheme 7 illustrates a method for preparing compounds of Formula I. Compound 7-1 with a displaceable leaving group (such as chloro, for example) in the presence of a suitable base (such Cs₂CO₃, NaH, ^{t}BuOK, KHMDS) is treated with compound 1-2 to provide a product of formula 7-2. Then compound of formula 7-2 was halogenated to provide compound of formula 7-3 wherein R¹⁴ is a halogen, such as Br or I. Compound of formula 7-3 may be subjected to transformations in a variety of ways known to those skilled in the art, for example such as treatment with TMSN₃ with copper catalyst, or metal-catalyzed coupling reaction to form carbon-carbon bond, or amine.

### Examples of Experimental Procedures:

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate. Products were generally dried undervacuum before being carried on to further reactions or submitted for biological testing. Mass spectrometry data is reported from liquid chromatography-mass spectrometry (LCMS) instrumentation. Mass spectra, MS (m/z), were recorded using either electrospray ionization (ESI) or atmospheric pressure chemical ionization (APCI). Where relevant and unless otherwise stated the m/z data provided are for isotopes 19F, 35Cl, 79Br and 127I. Chemical shifts for nuclear magnetic resonance (NMR) data are expressed in parts per million (ppm, 5) referenced to residual peaks from the deuterated solvents employed, using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d6-DMSO, derterodimethylsulphoxide; and CD₃OD, deuteromethanol.

In general, reactions were followed by thin layer chromatography (TLC) and/or liquid chromatography-mass spectrometry (LCMS), and subjected to work-up when appropriate. Purification was carried out by chromatographic and/or HPLC. Unless noted otherwise, all reactants were obtained commercially.

### Example 1

### Preparation of 7-methoxy-1-((5-oxopyrrolidin-2-yl)methoxy)isoquinolin-6-yl carbamate (II)

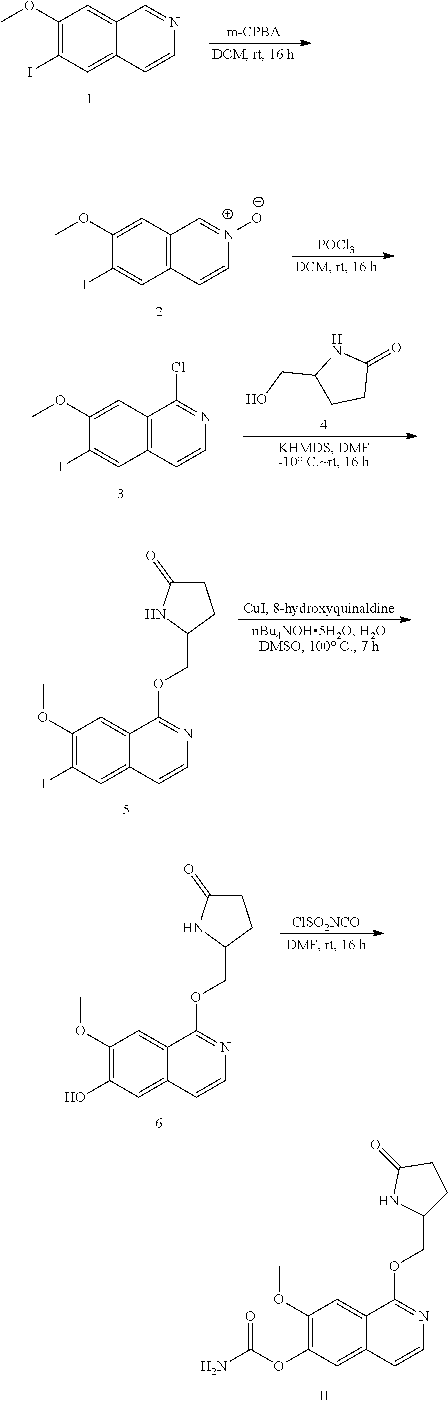

### Step 1: Preparation of 6-iodo-7-methoxyisoquinoline 2-oxide (2)

To a solution of compound 1 (300 mg) in DCM (20 mL) was added m-CPBA (272 mg) at rt. The mixture was stirred 3 h before it was washed with sat. NaHCO₃ and Na₂S₂O₃. DCM layer was dried with anhydrous Na₂SO₄, and then concentrated to give crude compound 2 (400 mg, yield 100%).

### Step 2: Preparation of 1-chloro-6-iodo-7-methoxyisoquinoline (3)

To a solution of compound 2 (2.6 g) in DCM (50 mL) at rt was added POCl₃ (1.3 g) slowly. The resulting mixture was stirred at rt for 16 h, followed by the standard work up procedure to give compound 3 (1.18 g, yield 43%) as a white solid. LC-MS: [M+H]⁺ 320.

### Step 3: Preparation of 5-(((6-iodo-7-methoxyisoquinolin-1-yl)oxy)methyl)pyrrolidin-2-one (5)

To a solution of compound 3 (1.5 g) and compound 4 (1.08 g) in dry DMF (100 mL) at −10° C. was added KHMDS (18.8 mL, 1 M). The resulting mixture was stirred at rt for 16 h before it was quenched with Sat. NH₄Cl. The mixture was extracted with DCM. The organic layer was dried over Na₂SO₄ and concentrated. The residue was triturated with PE/EA. The solid was collected and dried to provide compound 5 (700 mg, yield 37%) as a yellow solid. LC-MS: [M+H]⁺ 302.

### Step 4: Preparation of 5-(((6-hydroxy-7-methoxyisoquinolin-1-yl) oxy) methyl) pyrrolidin-2-one (6)

To a solution of compound 1 (500 mg, Example 1, compound 5) in DMSO/H₂O (4 mL/6 mL) was added CuI (24 mg), ⁿBu₄NOH.5H₂O (2.44 g) and 8-hydroxyquinaldine (40 mg). The reaction mixture was stirred at 90° C. for 6 h under Ar, followed by the standard work up procedure to provide compound 6 (160 mg, 44% yield) as a yellow oil.

### Step 5: Preparation of 7-methoxy-1-((5-oxopyrrolidin-2-yl) methoxy) isoquinolin-6-yl carbamate (II)

To a solution of compound 6 (140 mg) in DMF (3 mL) was added CIS₂NCO (137 mg) at 0° C. The reaction was stirred at rt for 16 h under Ar, followed by the standard work up procedure to give desired compound (II) as a white solid (17 mg, 10%). LC-MS: [M+H]⁺ 332.1 ¹H NMR (400 MHz, DMSO-d₆) δ 10.13 (br s, 1H), 7.91 (br s, 1H), 7.75 (d, J=5.6 Hz, 1H), 7.43 (br s, 1H), 7.30 (s, 1H), 7.14 (d, J=5.6 Hz, 1H), 7.11 (s, 1H), 4.75-4.64 (m, 2H), 4.57-4.49 (m, 1H), 3.89 (s, 3H), 2.96-2.82 (m, 1H), 2.64-2.55 (m, 1H), 2.34-2.26 (m, 1H), 2.10-1.99 (m, 1H).

### Example 2

### Preparation of N-hydroxy-7-methoxy-1-((5-oxopyrrolidin-2-yl) methoxy) isoquinoline-6-carboxamide (IV)

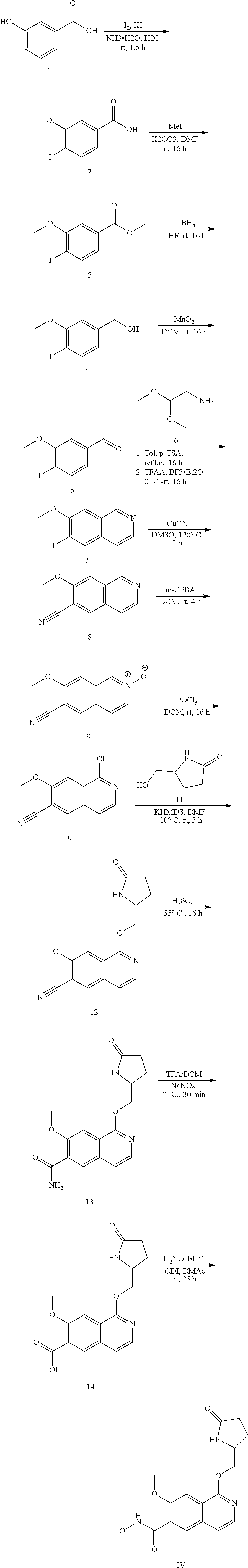

### Step 1: Preparation of 3-hydroxy-4-iodobenzoic acid (2)

To a solution of compound 1 (200 g) in NH₃.H₂O (2.9 L) was added I₂ (338 g) in portions and KI (264 g) in H₂O (1.5 L) by funnel. The mixture was stirred at rt for 1.5 h. To the mixture was added con. HCl slowly at 20-35° C. and the mixture was stirred for 10 min (pH<2). The precipitate was collected and recrystallized with ethanol/H₂O to give crude compound 2 (141 g, yield 37%) as a slightly yellow solid.

### Step 2: Preparation of methyl 4-iodo-3-methoxybenzoate (3)

To a solution of compound 2 (140 g) in DMF (400 mL) was added K₂CO₃ (293 g). The reaction mixture was cooled to 0° C. and MeI (301 g) was added. When the reaction is finished, solid was removed and water was added. Compound 3 (125 g, yield 81%) was obtained as a yellow solid after the standard work up procedure.

### Step 3: Preparation of (4-iodo-3-methoxyphenyl)methanol (4)

To a solution of LiBH₄ (23.0 g) in THF (400 mL) was added dropwise compound 3 (155 g) in THF (400 mL) at rt. The reaction mixture was stirred at rt overnight. After the standard work up procedure, compound 4 (128 g, yield 89%) was obtained as a white solid.

### Step 4: Preparation of 4-iodo-3-methoxybenzaldehyde (5)

To a solution of compound 4 (128 g) in DCM (500 mL) was added MnO₂ (337 g). The reaction mixture was stirred at rt for 16 h under Ar. The mixture was filtered and the filtrate was evaporated to give compound 5 (120 g, yield 94%) as a yellow solid.

### Step 5: Preparation of 6-iodo-7-methoxyisoquinoline (7)

To a mixture of compound 5 (104 g) in toluene (1 L) was added compound 6 (50.0 g) and p-TSA (6.8 g). The mixture was stirred at 150° C. using a Dean-stark strap for 16 h before it was cooled to 0° C. TFAA (250 g) followed by BF₃.Et₂O (169 g) were added dropwise at 0° C. The mixture was stirred at rt for 16 h before it was poured into 2 M HCl solution. The precipitate was suspended in EtOAc and saturated Na₂CO₃ solution. The organic layer was washed with brine, dried over Na₂SO₄ and concentrated to give compound 7 (46.5 g, yield 40%) as a white solid.

### Step 6: Preparation of 7-methoxyisoquinoline-6-carbonitrile (8)

To a solution of compound 7 (3.0 g) in DMSO (30 mL) was added CuCN (2.07 g). The reaction mixture was stirred at 120° C. for 16 h. EtOAc (40 mL) was added, and the insoluble solid was suspended in NH₃.H₂O (40 mL) and EtOAc. The insoluble solid in water phase was collected, washed with DCM/MeOH (9/1), and the insoluble solid was filtered off. The combined organic extracts were washed with brine, dried over Na₂SO₄, and concentrated in vacuo to provide compound 8 (1.8 g, yield 93%) as a yellow solid.

### Step 7: Preparation of 6-cyano-7-methoxyisoquinoline 2-oxide (9)

The title compound was synthesized using the same method as Example 1 step 1, except Compound 8 was used.

### Step 8: Preparation of 1-chloro-7-methoxyisoquinoline-6-carbonitrile (10)

The title compound was synthesized using the same method in Example 1 step 2, except compound 9 was used.

### Step 9: Preparation of 7-methoxy-1-((5-oxopyrrolidin-2-yl) methoxy) isoquinoline-6-carbonitrile (12)

The title compound was synthesized using the same method as Example 1 step 3, except compound 10 was used.

### Step 10: Preparation of 7-methoxy-1-((5-oxopyrrolidin-2-yl) methoxy) isoquinoline-6-carboxamide (13)

A solution of compound 12 (600 mg) in H₂SO₄ (6 mL) was stirred at 55° C. for 16 h. The reaction mixture was cooled to rt before it was added dropwise to 30 mL ice-cold con. NH₃.H₂O. The precipitated solid was collected, dissolved in DCM/MeOH (10/1), filtered again and the filtrate was concentrated to give compound 13 (610 mg, yield 96%) as a yellow solid.

### Step 11: Preparation of 7-methoxy-1-((5-oxopyrrolidin-2-yl) methoxy) isoquinoline-6-carboxylic acid (14)

To a solution of compound 13 (300 mg) in TFA/DCM (10 mL/2.5 mL) at 0° C. was added NaNO₂ (330 mg). The reaction mixture was stirred at 0° C. for 30 min before it was quenched by ice-water. Compound 14 (400 mg, yield 100%) was obtained after the standard work up procedure.

### Step 12: Preparation of N-hydroxy-7-methoxy-1-((5-oxopyrrolidin-2-yl) methoxy) isoquinoline-6-carboxamide (IV)

To a solution of compound 14 (200 mg) in DMAc (2.5 mL) was added CDI (189 mg). NH₂OH.HCl (162 mg) was added 1 h later, and the mixture was stirred at rt for 24 h. The reaction solution was directly purified by prep-HPLC to give desired compound (33 mg, yield 16%) as white solid. LC-MS: [M+H]⁺ 332. ¹H NMR (400 MHz, DMSO-d₆) δ 10.80 (br s, 1H), 9.29 (br s, 1H), 8.15 (s, 1H), 7.98 (s, 1H), 7.90 (d, J=5.6 Hz, 1H), 7.62 (s, 1H), 7.42 (d, J=5.6 Hz, 1H), 4.51-4.44 (m, 1H), 4.30-4.23 (m, 1H), 4.03 (br s, 1H), 3.95 (s, 3H), 2.38-2.11 (m, 4H), 1.92-1.88 (m, 1H).

### Example 3

### Preparation of 7-methoxy-1-(((5-oxopyrrolidin-2-yl) methyl) amino) isoquinoline-6-carboxamide (VII)

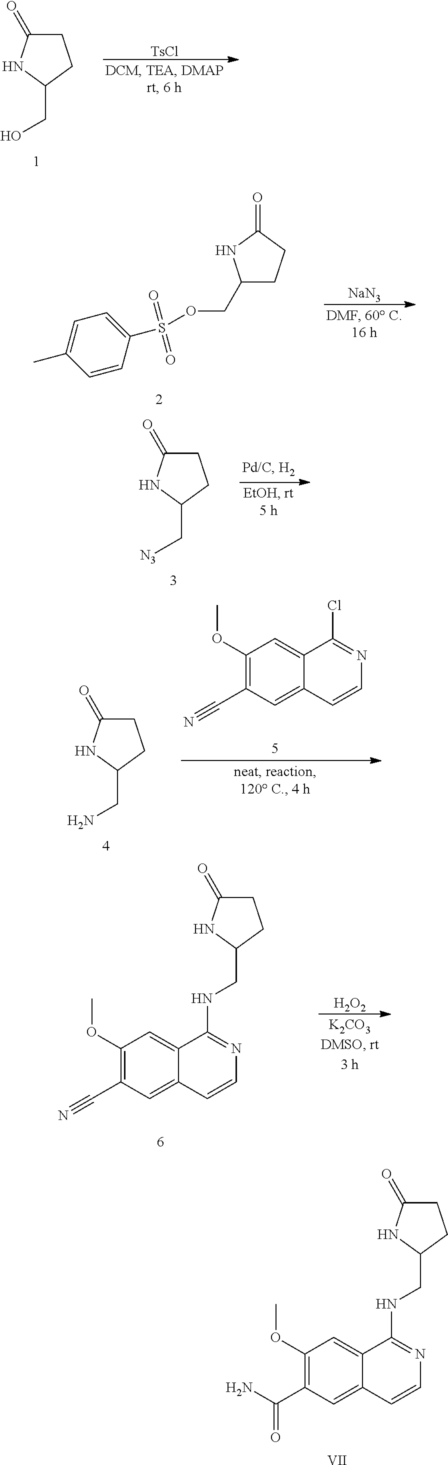

### Step 1: Preparation of (5-oxopyrrolidin-2-yl) methyl 4-methylbenzenesulfonate (2)

To the mixture of compound 1 (5.0 g) and TsCl (10.6 g) in DCM (10 mL) were added DMAP (1.1 g) and TEA (5.6 g) under 0° C. The mixture was stirred at 0° C. for 0.5 h before it was warmed to rt. The mixture was stirred at rt for 6 h, followed by standard work up procedure to give compound 2 as a white solid (9.0 g, yield 77%).

### Step 2: Preparation of 5-(azidomethyl) pyrrolidin-2-one (3)

To a solution of compound 2 (4.0 g) in DMF (30 mL) was added NaN₃ (1.44 g). The mixture was stirred at 60° C. under N₂ overnight. After cooled to rt, DCM was added to the mixture. After standard work up procedure, compound 3 was obtained as a colorless oil (1.97 g, yield 95%).

### Step 3: Preparation of 5-(aminomethyl) pyrrolidin-2-one (4)

To a solution of compound 3 (1.97 g) in EtOH (55 mL) was added Pd/C (10% wt, 400 mg). The resulting mixture was stirred at rt under 1 atm of H₂ atmosphere for 5 h. The mixture was filtered and the filtrate was concentrated and purified to give compound 4 as a yellow oil (620 mg, yield 15%).

### Step 4: Preparation of 7-methoxy-1-(((5-oxopyrrolidin-2-yl) methyl) amino) isoquinoline-6-carbonitrile (6)

The mixture of compound 5 (600 mg) and compound 4 (558 mg) in CH₃CN (3.5 mL) was stirred at 90° C. for 20 min. The solvent was removed and the residue was heated at 120° C. under N₂ atmosphere for 4 h. The purification was carried by column chromatography to give compound 6 as a yellow solid (130 mg, yield 8%)

### Step 5: Preparation of 7-methoxy-1-(((5-oxopyrrolidin-2-yl) methyl) amino) isoquinoline-6-carboxamide (VII)

To a solution of compound 6 (200 mg) in DMSO (3 mL) was added K₂CO₃ (466 mg) and H₂O₂ (30%, 780 uL). The mixture was stirred under N₂ atmosphere for 3 h before it was quenched by Me₂S (1320 uL). EA was added and the mixture was filtered. The filtrate was concentrated, and then purified by prep-HPLC to give compound 7 as a yellow solid (100 mg, yield 47%). LC-MS: [M+H]⁺ 315

¹H NMR (400 MHz, DMSO) δ 8.03 (s, 1H), 7.81-7.77 (m, 3H), 7.68 (s, 1H), 7.66 (br s, 1H), 7.46 (t, J=5.6 Hz, 1H), 6.94 (d, J=6.0 Hz, 1H), 3.99 (s, 3H), 3.96-3.85 (m, 1H), 3.61-3.46 (m, 2H), 2.28-2.03 (m, 3H), 1.93-1.78 (m, 1H).

### Example 4

### Preparation of 4⁵-oxo-2,7,10-trioxa-1(1,7)-isoquinolina-4(2,1)-pyrrolidinacyclodecaphane-1⁶-carboxamide (IX)

### Step 1: Preparation of 2-(2-chloroethoxy)ethyl 4-methylbenzenesulfonate (2)

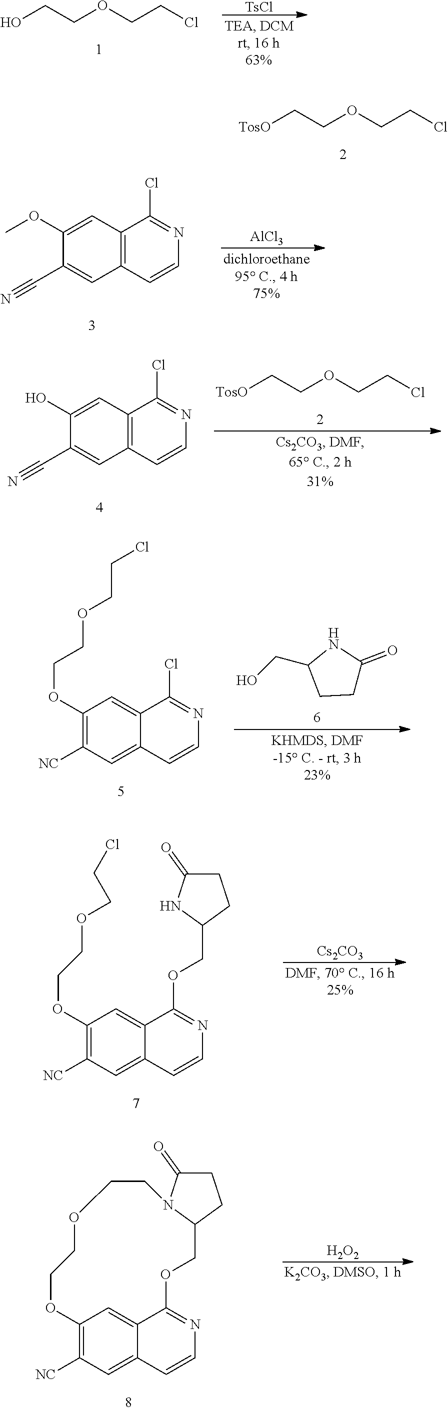

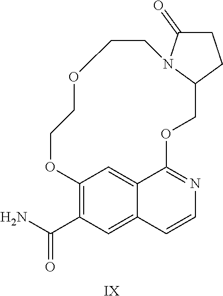

Compound 2 (1.4 g, yield 63%, colorless oil) was prepared in the same manner as compound 2 in example 3, except compound 1 was used.

### Step 2: Preparation of 1-chloro-7-hydroxyisoquinoline-6-carbonitrile (4)

To a solution of compound 3 (1.7 g) in dichloroethane was added AlCl₃ (970 mg) at rt. The mixture was stirred at 95° C. for 4 h before it was quenched by water. Compound 4 (1.2 g, yield 75%) was obtained after standard work up procedure as a yellow solid. LC-MS: [M+H]⁺ 205.

### Step 3: Preparation of 1-chloro-7-(2-(2-chloroethoxy)ethoxy)isoquinoline-6-carbonitrile (5)

To a solution of compound 4 (1.5 g) and compound 2 (2.0 g) in DMF was added Cs₂CO₃ (4.80 g) at rt. The mixture was stirred at 65° C. for 2 h. Compound 5 (700 mg, yield 31%) as a yellow solid was obtained after standard work up procedure. LC-MS: 311 [M+H]⁺.

### Step 4: Preparation of 7-(2-(2-chloroethoxy)ethoxy)-1-((5-oxopyrrolidin-2-yl)methoxy)isoquinoline-6-carbonitrile (7)

Compound 7 (200 mg, yield 23%) as a yellow solid was prepared in the same manner as compound 12 in example 2, except compound 5 was used. LC-MS: [M+H]⁺ 390

### Step 5: Preparation of 4⁵-oxo-2,7,10-trioxa-1(1,7)-isoquinolina-4(2,1) pyrrolidinacyclodecaphane-1⁶-carbonitrile (8)

To a solution of compound 7 (200 mg) in anhydrous DMF (50 mL) at rt was added Cs₂CO₃ (334 mg). The resulting mixture was stirred at 70° C. for 16 h. Compound 8 (45 mg, yield 25%) was obtained after standard work up procedure as a yellow solid. LC-MS: [M+H]⁺ 354

### Step 6: Preparation of 4⁵-oxo-2,7,10-trioxa-1(1,7)-isoquinolina-4(2,1)-pyrrolidinacyclodecaphane-1⁶-carboxamide (IX)

Compound IX (28 mg, yield 59%) was synthesized in the same manner as compound VII. LC-MS: [M+H]⁺ 372. ¹H NMR (400 MHz, CD₃OD) δ 8.31 (d, 1H), 7.97 (br s, 2H), 7.89 (s, 1H), 7.64 (s, 1H), 7.02 (d, 1H), 4.26 (m, 1H), 4.11 (t, 2H), 3.90-4.01 (m, 2H), 3.79 (t, 2H), 3.65 (m, 2H), 3.30-3.42 (m, 2H), 2.06-2.23 (m, 3H), 1.81 (m, 1H).

### Example 5

### Preparation of (S)-7-methoxy-1-((5-oxopyrrolidin-2-yl)methoxy)isoquinoline-6-carbothioamide (XI)

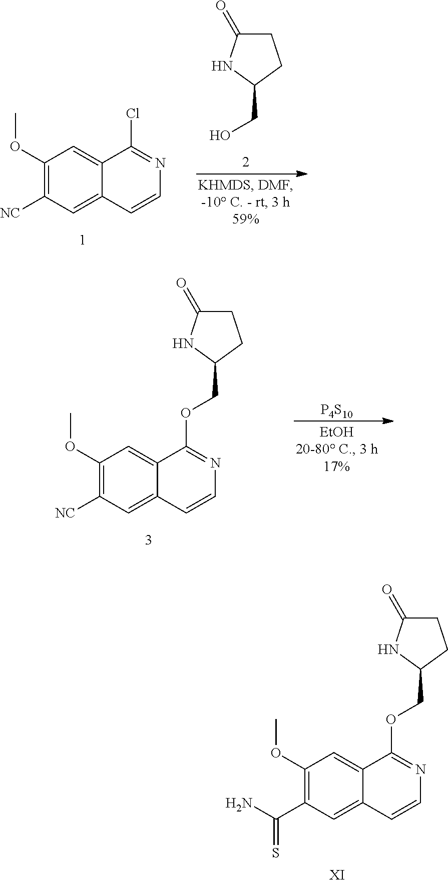

### Step 1: Preparation of (S)-7-methoxy-1-((5-oxopyrrolidin-2-yl) methoxy) isoquinoline-6-carbonitrile (3)

Compound 3 (400 mg, yield 59%) as a light yellow solid was prepared in the same manner as compound 12 in example 2, except compound 2 was used.

### Step 2: (S)-7-methoxy-1-((5-oxopyrrolidin-2-yl) methoxy) isoquinoline-6-carbothioamide (XI)

A solution of P₄S₁₀ (2.1 g) in EtOH was stirred at rt for 0.5 h. Compound 3 (140 mg) was added and the resulting solution was stirred at 80° C. for 2.5 h. The reaction solution was concentrated and purified by prep-HPLC to give desired compound XI (27 mg, 17%) as a white solid. LC-MS: 332 [M+H]⁺. ¹H NMR (400 MHz, DMSO-d₆) 10.16 (br s, 1H), 9.58 (br s, 1H), 8.14 (s, 1H), 7.94 (s, 1H), 7.88 (d, J=5.6 Hz, 1H), 7.58 (s, 1H), 7.39 (d, J=6.0 Hz, 1H), 4.51-4.47 (m, 1H), 4.31-4.27 (m, 1H), 4.07-4.01 (m, 1H), 3.94 (s, 3H), 2.34-2.17 (m, 3H), 1.92-1.87 (m, 1H).

### Example 6

### Preparation of (S)-7-methoxy-1-((5-oxopyrrolidin-2-yl) methoxy) isoquinoline-6-carbothioic S-acid (XIII)

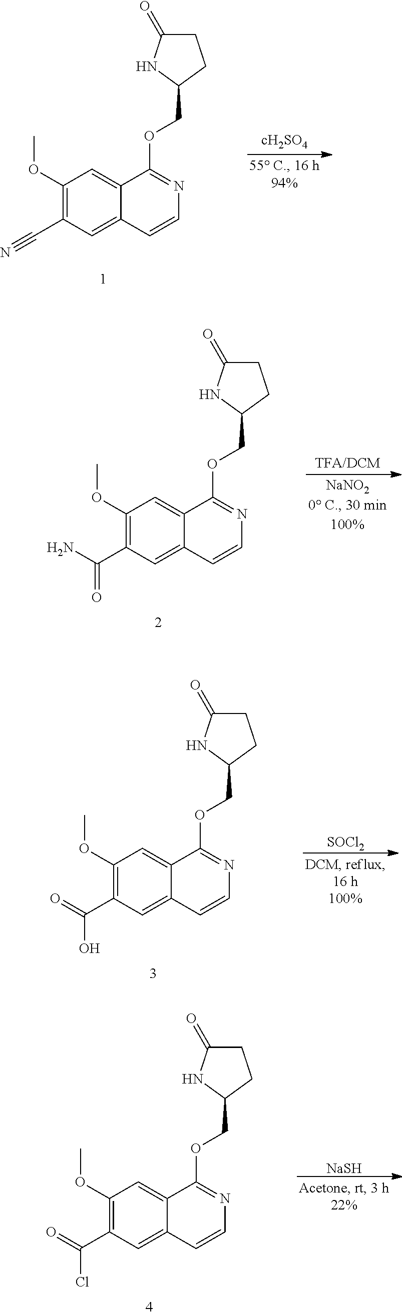 [SMILES: COc1cc2c(OC[C@@H]3CCC(=O)N3)nccc2cc1C#N ; COc1cc2c(OC[C@@H]3CCC(=O)N3)nccc2cc1C(N)=O ; COc1cc2c(OC[C@@H]3CCC(=O)N3)nccc2cc1C(=O)O ; "SOCl2" ; COc1cc2c(OC[C@@H]3CCC(=O)N3)nccc2cc1C(=O)Cl ; "NaSH"]

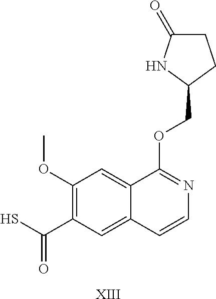

### Step 1: Preparation of (S)-7-methoxy-1-((5-oxopyrrolidin-2-yl) methoxy) isoquinoline-6-carboxamide (2)

Compound 2 (200 mg, yield 94%) as a yellow solid was synthesized in the same manner as compound 13 in example 2, except compound 1 was used.

### Step 2: Preparation of (S)-7-methoxy-1-((5-oxopyrrolidin-2-yl) methoxy) isoquinoline-6-carboxylic acid (3)

Compound 3 (265 mg, yield 100%) as a yellow solid was prepared in the same manner as compound 14 in example 2, except compound 2 was used.

### Step 3: Preparation of (S)-7-methoxy-1-((5-oxopyrrolidin-2-yl) methoxy) isoquinoline-6-carbonyl chloride (4)

A solution of Compound 3 (150 mg) and SOCl₂ (2.5 mL) was stirred at reflux for 16 h. Solvent was removed to give compound 4 (180 mg, yield 100%) as a colorless gum. LC-MS: [M+H]⁺ 331.

### Step 4: Preparation of (S)-7-methoxy-1-((5-oxopyrrolidin-2-yl) methoxy) isoquinoline-6-carbothioic S-acid (XIII)

To a solution of NaSH (450 mg) in H₂O (5 mL) was added compound 4 (180 mg) in acetone (25 mL). The reaction mixture was stirred at rt for 3 h. The reaction solution was concentrated and purified by prep-HPLC to give desired compound XIII (40 mg, yield 22%) as a yellow solid.

LC-MS: [M+H]⁺ 333. ¹H NMR (400 MHz, DMSO-d₆) δ 8.11 (s, 1H), 7.77 (d, J=5.6 Hz, 1H), 7.47 (s, 1H), 7.40 (s, 1H), 7.24 (d, J=6.0 Hz, 1H), 4.45-4.41 (m, 1H), 4.31-4.27 (m, 1H), 4.04-4.01 (m, 1H), 3.81 (s, 3H), 2.35-2.16 (m, 3H), 1.92-1.87 (m, 1H).

### Example 7

### Preparation of 5-oxo-2,7,10-trioxa-1(1,7)-isoquinolina-4(3,1)-azetidinacyclodecaphane-16-carboxamide (XIV)

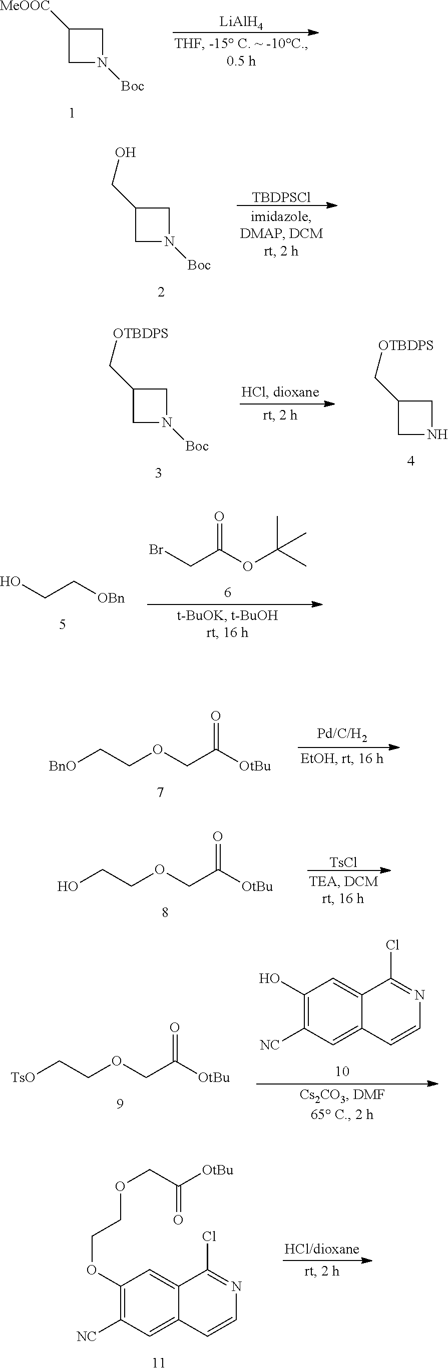

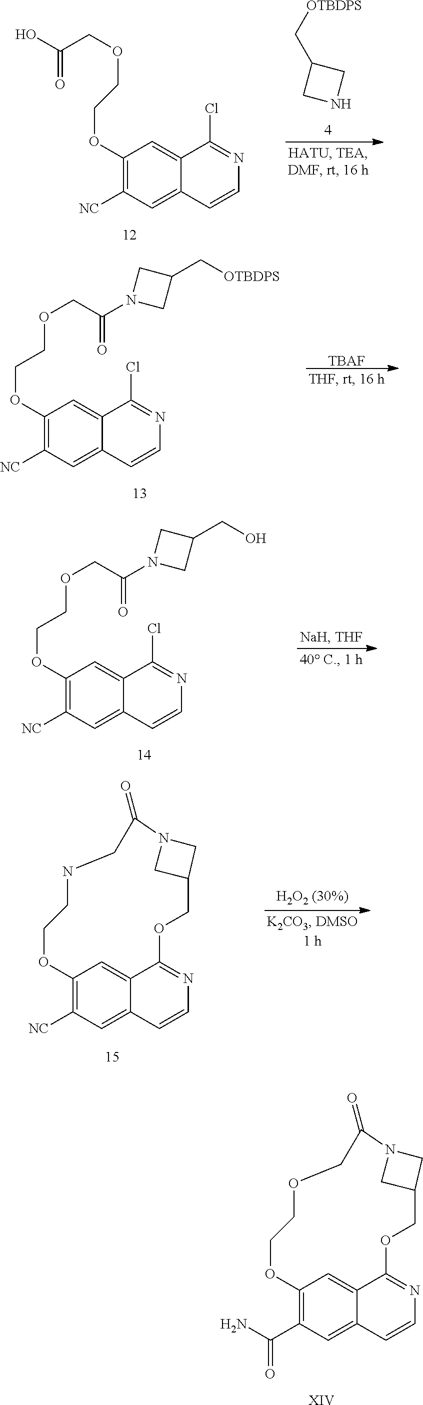

### Step 1: preparation of tert-butyl 3-(hydroxymethyl) azetidine-1-carboxylate (2)

To a solution of compound 1 (2.5 g) in anhydrous THF (30 mL) was added LiAH₄ (883 mg) at −15° C. The mixture was stirred at −15° C.-10° C. for 0.5 h, followed by standard work up procedure to give compound 2 (1.5 g, yield 69%) as a colorless oil.

### Step 2: preparation of tert-butyl 3-(((tert-butyldiphenylsilyl) oxy) methyl) azetidine-1-carboxylate (3)

To a solution of compound 2 (3.4 g), imidazole (2.7 g) and DMAP (222 mg) in anhydrous DCM (50 mL) was added dropwise TBDPSCl (5.2 g) at rt. The mixture was stirred at rt for 2 h. Compound 3 (6.5 g, yield 84%) was obtained after standard work up procedure.

### Step 3: preparation of 3-(((tert-butyldiphenylsilyl)oxy)methyl)azetidine hydrochloride (4)

Compound 3 (5.5 g) was dissolved in HCl/dioxane (4 mol/L in dioxane, 50 mL) at rt. The mixture was stirred at rt for 2 h, and then it was concentrated. The residue was dissolved in DCM, and the pH of the solution was adjusted to >7 with TEA. Solvent was removed and the residue was purified by flash column chromatography to give compound 4 (2.3 g, yield 55%) as a colorless oil.

### Step 4: preparation of tert-butyl 2-(2-(benzyloxy)ethoxy)acetate (7)

To a solution of compound 5 (10.0 g) in ^{t}BuOH (100 mL) was added ^{t}BuOK (8.1 g) at rt. The mixture was stirred at rt for 0.5 h, and then compound 6 (12.8 g) was added. The mixture was stirred at rt for 16 h. Compound 7 (7.0 g, yield 40%) was obtained after standard work up procedure.

### Step 5: preparation of tert-butyl 2-(2-hydroxyethoxy) acetate (8)

To a solution of compound 7 (7 g) in ethanol (70 mL) was added 10% Pd/C (700 mg) at rt. The mixture was stirred at rt for 16 h under H₂. The mixture was filtered and the filtrate was concentrated under the reduced pressure to give compound 8 (4.0 g, yield 87%) as a colorless oil.

### Step 6: preparation of tert-butyl 2-(2-(tosyloxy)ethoxy)acetate (9)

Compound 9 (2.7 g, yield 36%) as a colorless oil was prepared in the same manner as compound 2 in example 4, except compound 8 was used.

### Step 7: preparation of tert-butyl 2-(2-((1-chloro-6-cyanoisoquinolin-7-yl) oxy)ethoxy)acetate (11)

The title compound (2.1 g, yield 77%) as a white solid was synthesized using same method as compound 5 in example 4, except compound 9 was used.

### Step 8: preparation of 2-(2-((1-chloro-6-cyanoisoquinolin-7-yl)oxy)ethoxy)acetic acid (12)

Compound 11 (1.4 g) was dissolved in HCl/dioxane (4 mol/L in dioxane, 10 mL) at rt. The mixture was stirred at rt for 2 h. The precipitation was collected, and dried to give compound 12 (1.0 g, yield 85%) as a yellow solid.

### Step 9: preparation of 7-(2-(2-(3-(((tert-butyldiphenylsilyl)oxy)methyl)azetidin-1-yl)-2-oxoethoxy)ethoxy)-1-chloroisoquinoline-6-carbonitrile (13)

To a solution of compound 12 (200 mg), TEA (265 mg) and HATU (497 mg) in DMF (10 mL) was added compound 4 (232 mg) at rt. The mixture was stirred at rt for 16 h. After standard work up procedure, compound 13 (230 mg, yield 58%) was obtained as a white solid.

### Step 10: preparation of 1-chloro-7-(2-(2-(3-(hydroxymethyl)azetidin-1-yl)-2-oxoethoxy)ethoxy)isoquinoline-6-carbonitrile (14)

To a solution of compound 13 (230 mg) in THF (50 mL) at rt was added TBAF (0.75 mL, 1 M solution in THF). The resulting mixture was stirred at rt for 16 h. After the standard work up procedure, compound 14 (45 mg, yield 32%) was obtained as a white solid.

### Step 11: Preparation of 5-oxo-2,7,10-trioxa-1(1,7)-isoquinolina-4(3,1)-azetidinacyclodecaphane-16-carbonitrile (15)

The tile compound 15 (12 mg, yield 44%, white solid) was synthesized according to example 4 step 5, except NaH and compound 14 were used.

### Step 12: Preparation of 5-oxo-2,7,10-trioxa-1(1,7)-isoquinolina-4(3,1)-azetidinacyclodecaphane-16-carboxamide (XIV)

Compound XIV was prepared in the same manner as compound IX. LC-MS: [M+H]⁺ 358.

¹H NMR (400 MHz, CD₃OD) δ 8.34 (s, 1H), 8.10 (s, 1H), 7.90 (d, J=5.6 Hz, 1H), 7.36 (d, J=5.6 Hz, 1H), 4.94-4.92 (m, 2H), 4.83-4.74 (m, 2H), 4.57-4.50 (m, 2H), 4.40-4.36 (m, 1H), 4.28-4.24 (m, 1H), 4.14-3.98 (m, 3H), 3.83 (d, J=13.2 Hz, 1H), 3.16-3.12 (m, 1H).

### Example 8

### Preparation of 1²-oxo-3,5,8-trioxa-1(4,3)-oxazolidina-4(1,7)-isoquinolinacyclononaphane-4⁶-carboxamide (XVII)

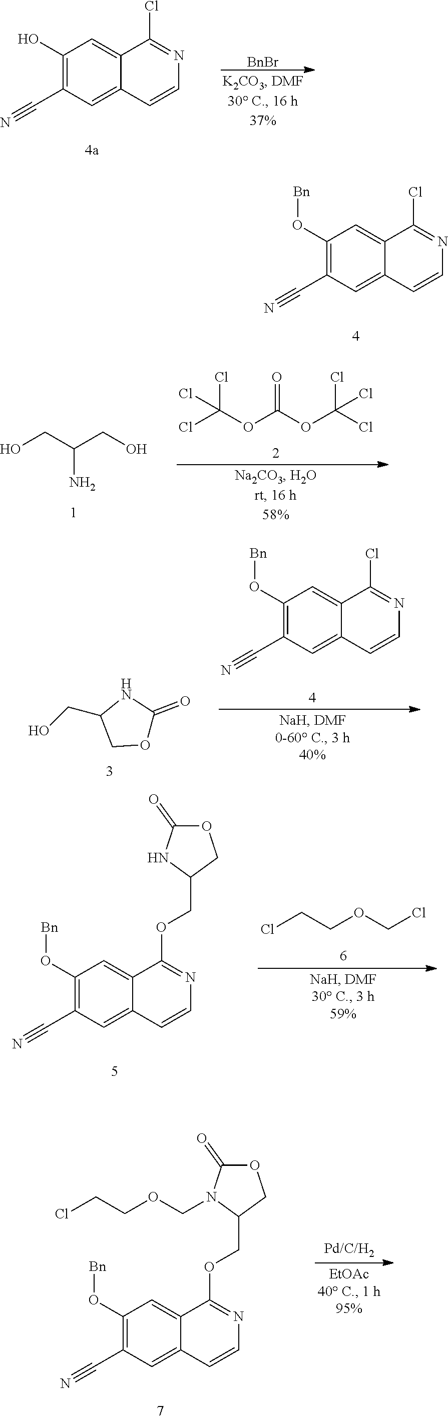

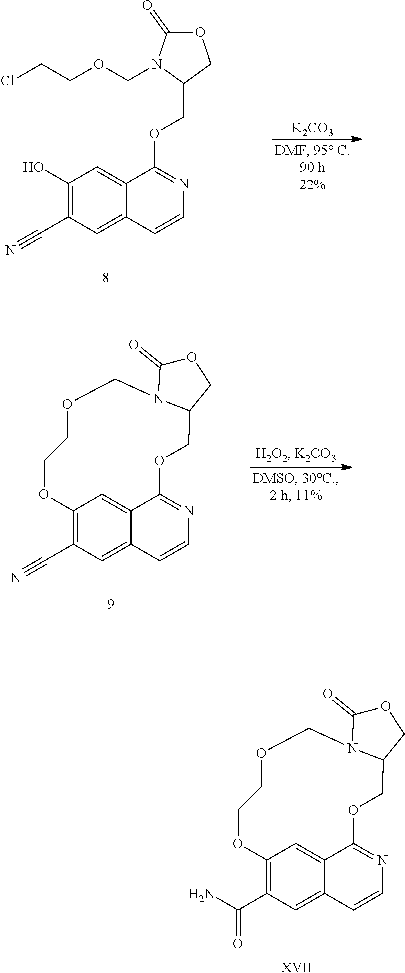

### Step 1: Preparation of 7-(benzyloxy)-1-chloroisoquinoline-6-carbonitrile (4)

To a solution of compound 4a (3.1 g) in DMF (30 mL) were added K₂CO₃ (8.4 g) and BnBr (10.4 g). The mixture was stirred at 30° C. for 16 h. Compound 4 (1.6 g, yield 37%) was obtained after standard work up procedure as a yellow solid.

### Step 2: preparation of 4-(hydroxymethyl) oxazolidin-2-one (3)

To a solution of compound 1 (20.0 g) in H₂O (100 mL) were added Na₂CO₃ (78 g) and compound 2 (21.5 g). The reaction mixture was stirred at rt for 16 h. Compound 3 (15.0 g, yield 58%) was obtained after standard work up procedure as a white solid.

### Step 3: preparation of 7-(benzyloxy)-1-((2-oxooxazolidin-4-yl)methoxy)isoquinoline-6-carbonitrile (5)

Compound 5 (51 mg, yield 40%) was prepared in the same manner as compound 7 in example 4, except compound 3, compound 4 and NaH were used.

### Step 4: preparation of 7-(benzyloxy)-1-((3-((2-chloroethoxy)methyl)-2-oxooxazolidin-4-yl)methoxy)isoquinoline-6-carbonitrile (7)

To a solution of NaH (15 mg, 60%) in DMF (1 mL) was added compound 5 (30 mg) at 0° C. The mixture was stirred at 0° C. for 10 min. Compound 6 (60 mg) was added and the solution was stirred at 30° C. for 3 h. Compound 7 (22 mg, yield 59%) was obtained after standard work up procedure as a gray solid.

### Step 5: preparation of 1-((3-((2-chloroethoxy)methyl)-2-oxooxazolidin-4-yl)methoxy)-7-hydroxyisoquinoline-6-carbonitrile (8)

A mixture of compound 7 (300 mg) and Pd/C (150 mg, 10%) in EtOAc (15 mL) was stirred at 40° C. for 1 h under H₂ atmosphere. The reaction mixture was filtered and concentrated to give compound 8 (230 mg, yield 95%) as a yellow solid.

### Step 6: Preparation of 1²-oxo-3,5,8-trioxa-1(4,3)-oxazolidina-4(1,7)-isoquinolinacyclononaphane-4⁶-carbonitrile (9)

A mixture of compound 8 (100 mg) and K₂CO₃ (110 mg) in DMF (10 mL) was stirred at 95° C. for 90 h under N₂ atmosphere. Compound 9 (20 mg, 22% yield) was obtained as a gray solid after standard work up procedure, and used as the crude.

### Step 7: Preparation of 12-oxo-3,5,8-trioxa-1(4,3)-oxazolidina-4(1,7)-isoquinolinacyclononaphane-46-carboxamide (XVII)

The title compound was synthesized using same method as Example 4 step 6, except compound 9 was used. LC-MS: [M+H]⁺ 360. ¹H NMR (400 MHz, CDCl₃) δ 8.71 (s, 1H), 8.52 (s, 1H), 8.00 (d, J=5.6 Hz, 1H), 7.78 (br s, 1H), 7.43 (d, J=5.6 Hz, 1H), 5.89 (br s, 1H), 4.98-4.92 (m, 1H), 4.88-4.86 (m, 1H), 4.79-4.73 (m, 2H), 4.63-4.59 (m, 1H), 4.55-4.51 (m, 1H), 4.45-4.39 (m, 2H), 4.22-4.15 (m, 2H), 3.81-3.76 (m, 1H).

### Example 9

### Preparation of (S)-4⁵-oxo-2,6,9-trioxa-1(1,7)-isoquinolina-4(2,1)-pyrrolidinacyclononaphane-1⁶-carboxamide (XVIII)

### Step 1: preparation of (S)-5-(((tert-butyldiphenylsilyl)oxy)methyl)pyrrolidin-2-one (2)

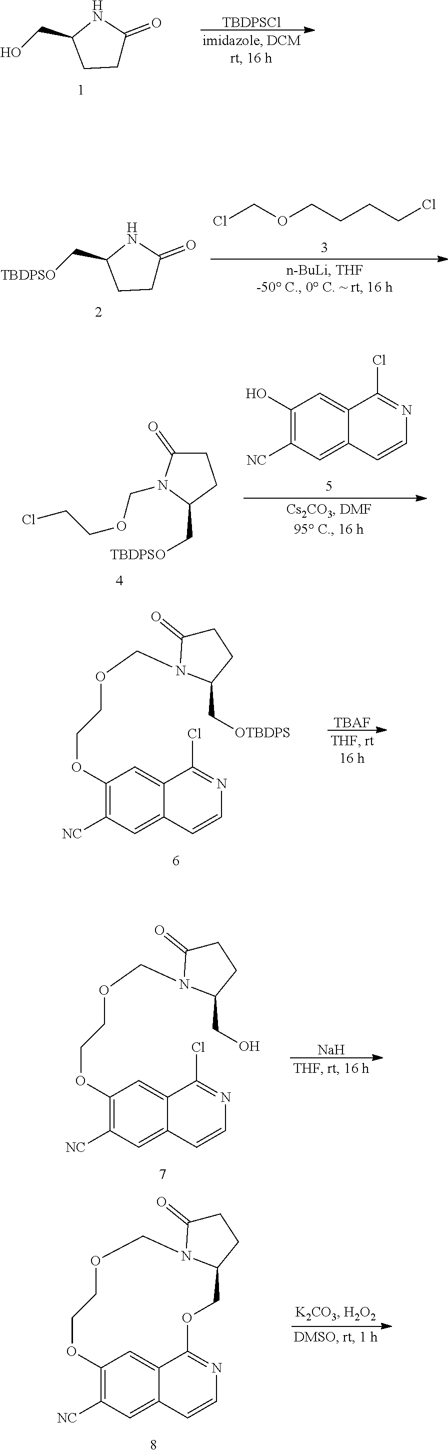

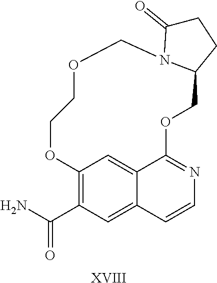

Compound 2 (2.5 g, yield 82%) was prepared in the same manner as compound 3 in example 7, except compound 1 was used.

### Step 2: preparation of (S)-5-(((tert-butyldiphenylsilyl)oxy)methyl)-1-((2-chloroethoxy)methyl)pyrrolidin-2-one (4)

To a solution of compound 2 (2.2 g) in anhydrous THF (50 mL) was added dropwise n-BuLi (1.6 mol/L in hexane, 4.78 mL) at −50° C. The mixture was stirred at −50 to −10° C. for 0.5 h, then compound 3 (884 mg) was added at −10° C. The mixture was stirred at rt for 16 h. Compound 4 (1.7 g, yield 61%) was obtained after standard work up procedure.

### Step 3: preparation of (S)-7-(2-((2-(((tert-butyldiphenylsilyl)oxy)methyl)-5-oxopyrrolidin-1-yl)methoxy)ethoxy)-1-chloroisoquinoline-6-carbonitrile (6)

The title compound was synthesized using the same method as Example 4 Step 3, except compound 4 was used.

### Step 4: preparation of (S)-1-chloro-7-(2-((2-(hydroxymethyl)-5-oxopyrrolidin-1-yl)methoxy)ethoxy)isoquinoline-6-carbonitrile (7)

The tile compound was synthesized using the same method in Example 7 Step 10, except compound 6 was used.

### Step 5: Preparation of (S)-4⁵-oxo-2,6,9-trioxa-1(1,7)-isoquinolina-4(2,1)-pyrrolidinacyclononaphane-1⁶-carbonitrile (8)

Compound 8 (173 mg, yield 48%) was synthesized in the same manner as compound 15 in example 7, except compound 7 was used.

### Step 6: Preparation of (S)-4⁵-oxo-2,6,9-trioxa-1(1,7)-isoquinolina-4(2,1)-pyrrolidinacyclononaphane-1⁶-carboxamide (XVIII)

The title compound was synthesized using the same method as Example 7 Step 12, except compound 8 was used. LC-MS: [M+H]⁺ 358. ¹H NMR (400 MHz, DMSO) δ 8.47 (s, 1H), 8.26 (s, 1H), 7.96 (d, J=5.6 Hz, 1H), 7.83 (br s, 1H), 7.74 (br s, 1H), 7.53 (d, J=6.0 Hz, 1H), 4.79-4.65 (m, 4H), 4.53-4.49 (m, 1H), 4.27-4.22 (m, 1H), 4.05-4.00 (m, 1H), 3.97-3.91 (m, 1H), 3.74-3.70 (m, 1H), 2.43-2.40 (m, 1H), 2.32-2.24 (m, 1H), 2.19-2.15 (m, 1H), 1.82-1.77 (m, 1H).

### Example 10

### Preparation of (4²S, 4³R)-4³-ethyl-4⁵-oxo-2,6,9-trioxa-1 (1,7)-isoquinolina-4 (2,1)-pyrrolidinacyclononaphane-1⁶-carboxamide (XIX)

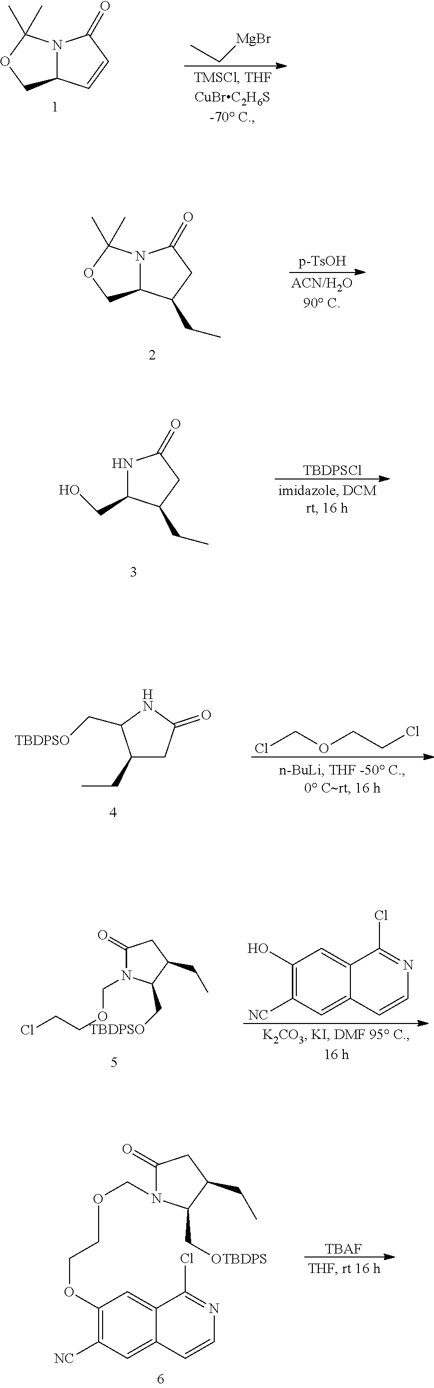

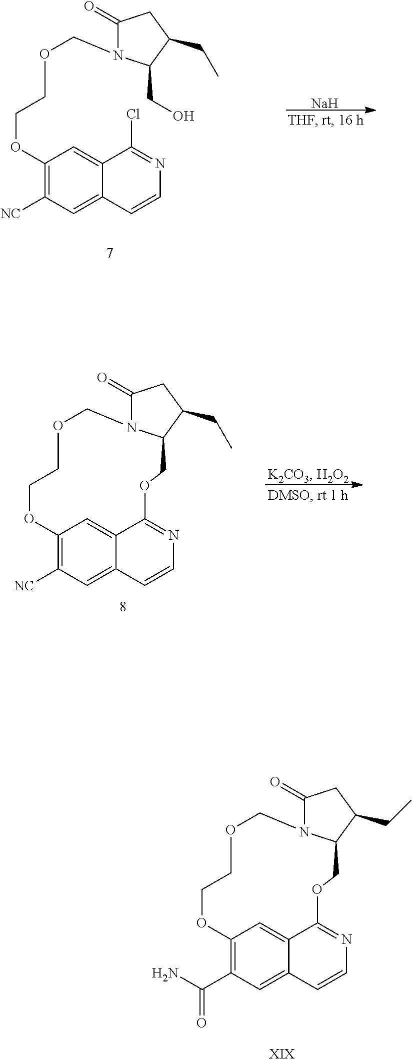

### Step 1: Synthesis of (7R)-7-ethyl-3, 3-dimethyltetrahydro-3H, 5H-pyrrolo[1,2-c]oxazol-5-one (2)

A suspension of cuprous bromide-dimethyl sulfide complex (10.3 g) and compound 1 (3 g) in THF (120 mL) was cooled to −70° C. and a solution of MgEtBr (33.3 mL, 3 M) was added slowly. The mixture was stirred for 20 min at −70° C. then TMSCl (6 mL) was added slowly. After the addition was complete, the mixture was maintained for another 1 h before being allowed to warm to room temperature. The title compound was obtained after standard work up procedure as a colorless oil (2.0 g, yield 57%).

### Step 2: Synthesis of (4R, 5S)-4-ethyl-5-(hydroxymethyl) pyrrolidin-2-one (3)

To a stirred solution of compound 2 (640 mg) in 8.1 mL CH₃CN and 0.9 mL of water was added p-TsOH (300 mg). The reaction mixture was heated at 90° C. for 2 h. The reaction mixture was cooled to r.t, concentrated, and the residue was purified by chromatography to give the title compound (430 mg, yield 86%).

### Step 3: Synthesis of (4R, 5S)-5-(((tert-butyldiphenylsilyl) oxy) methyl)-4-ethylpyrrolidin-2-one (4)

The title compound was synthesized using same method in Example 7 Step 2, except compound 3 was used.

### Step 4: Synthesis of (4R, 5S)-5-(((tert-butyldiphenylsilyl) oxy) methyl)-1-((2-chloroethoxy) methyl)-4-ethylpyrrolidin-2-one (5)

Compound 5 was synthesized in the same manner as compound 4 in example 10, except compound 4 was used.

### Step 5: Synthesis of 7-(2-(((2S, 3R)-2-(((tert-butyldiphenylsilyl) oxy) methyl)-3-ethyl-5-oxopyrrolidin-1-yl) methoxy) ethoxy)-1-chloroisoquinoline-6-carbonitrile (6)

Compound 6 (white solid, 330 mg, yield 90%) was synthesized in the same manner as compound 6 in example 10, except compound 5 was used.

### Step 6: Synthesis of 1-chloro-7-(2-(((2S, 3R)-3-ethyl-2-(hydroxymethyl)-5-oxopyrrolidin-1-yl) methoxy) ethoxy) isoquinoline-6-carbonitrile (7)

The title compound was synthesized using the same method as example 10 step 4, except compound 6 was used.

### Step 7: Synthesis of (4²S, 4³R)-4³-ethyl-4⁵-oxo-2, 6, 9-trioxa-1 (1, 7)-isoquinolina-4 (2,1)-pyrrolidinacyclononaphane-1⁶-carbonitrile (8)

Compound 8 (white solid, 115 mg, yield 70%) was prepared in the same manner as compound 8 in example 10, except compound 7 was used.

### Step 8: Synthesis of (4²S, 4³R)-4³-ethyl-4⁵-oxo-2, 6, 9-trioxa-1 (1,7)-isoquinolina-4 (2,1)-pyrrolidinacyclononaphane-1⁶-carboxamide (XIX)

The title compound was prepared in the same manner as compound XVIII, except compound 8 was used. LC-MS: [M+H]⁺ 386.1. ¹H NMR (400 MHz, DMSO) ¹H NMR (400 MHz, DMSO) δ 8.53 (s, 1H), 8.24 (s, 1H), 7.97 (d, J=5.9 Hz, 1H), 7.84 (s, 1H), 7.74 (s, 1H), 7.55 (d, J=5.9 Hz, 1H), 4.83 (d, J=9.6 Hz, 1H), 4.75 (dd, J=10.1, 4.3 Hz, 1H), 4.64-4.51 (m, 3H), 4.33 (dd, J=10.8, 6.7 Hz, 1H), 3.94 (t, J=6.1 Hz, 1H), 3.88 (s, 2H), 2.37-2.30 (m, 2H), 1.73-1.52 (m, 2H), 0.95 (t, J=7.3 Hz, 3H).

### Example 11

### Preparation of (S)-1-(3-aminopiperidin-1-yl)-7-methoxyisoquinoline-6-carboxamide (XX)

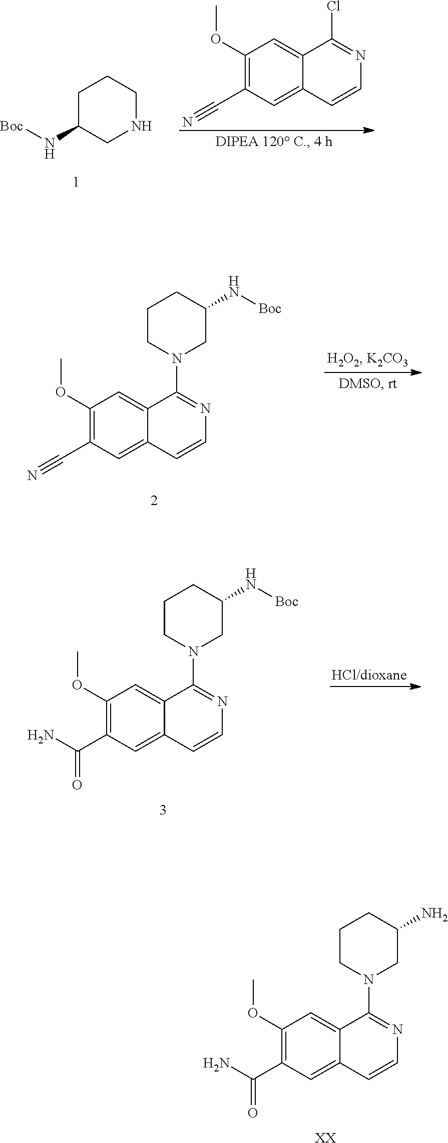

### Step 1: Synthesis of tert-butyl (S)-(1-(6-cyano-7-methoxyisoquinolin-1-yl) piperidin-3-yl) carbamate (2)

To a solution of 1-chloro-7-methoxyisoquinoline-6-carbonitrile (218 mg) in DIPEA (4.0 mL) was added compound 1 (300 mg). The mixture was stirred at 120° C. for 8 h. The title compound 2 was obtained as a yellow solid (170 mg, yield 45%) after standard work up procedure.

### Step 2: Synthesis of tert-butyl (S)-(1-(6-carbamoyl-7-methoxyisoquinolin-1-yl) piperidin-3-yl) carbamate (3)

Compound 3 (yellow solid, 81 mg, yield: 77%) was prepared in the same manner as compound XVIII, except compound 2 was used.

### Step 3: Synthesis of (S)-1-(3-aminopiperidin-1-yl)-7-methoxyisoquinoline-6-carboxamide (XX)

A mixture of compound 3 (81 mg) in 4M HCl/dioxane (3 mL) was stirred for 2 h at rt. The title compound XX was obtained (23 mg, yield 38%) after standard work up procedure.

LC-MS: [M+H]⁺ 301.1. ¹H NMR (400 MHz, CDCl₃) δ 8.58 (s, 1H), 8.19-8.11 (m, 1H), 7.83 (brs, 1H), 7.58 (s, 2H), 7.39-7.33 (m, 1H), 6.04 (brs, 1H), 4.06 (s, 3H), 3.61-3.48 (m, 2H), 3.25-2.89 (m, 3H), 2.05-1.81 (m, 2H), 1.48-1.27 (m, 2H).

### Example 12

### Preparation of (S)-1-(3-hydroxypiperidin-1-yl)-7-methoxyisoquinoline-6-carboxamide (XXI)

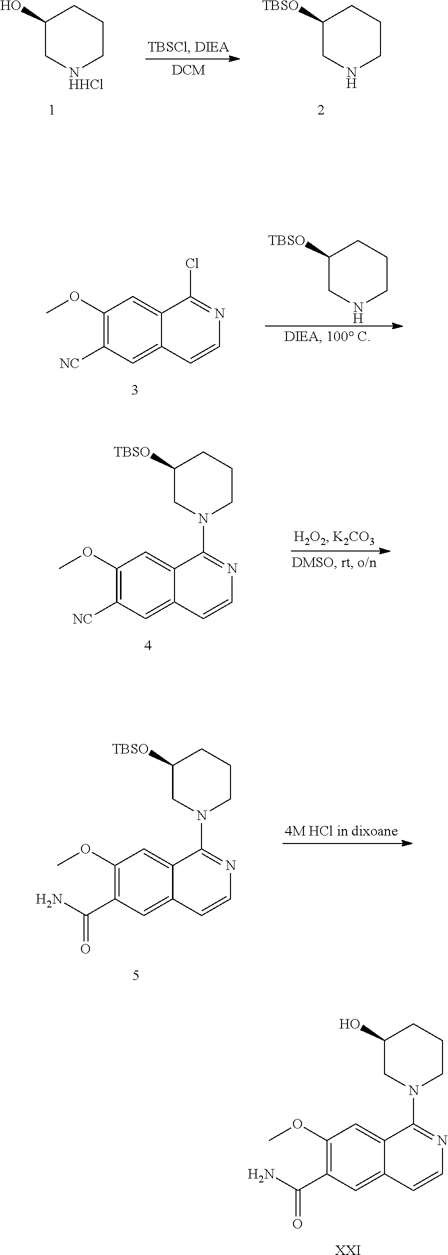

### Step 1: Synthesis of (S)-3-((tert-butyldimethylsilyl) oxy) piperidine (2)

Compound 2 (1.6 g, yield 51%) was prepared in the same manner as compound 4 in example 10, except compound 1 and TBSCl were used.

### Step 2: Synthesis of (S)-1-(3-((tert-butyldimethylsilyl) oxy) piperidin-1-yl)-7-methoxyisoquinoline-6-carbonitrile (4)

Compound 4 (colorless solid, 340 mg, yield 62%) was prepared in the sample manner as compound 2 in example 11, except compound 2 was used.

### Step 3: Synthesis of (S)-1-(3-((tert-butyldimethylsilyl) oxy) piperidin-1-yl)-7-methoxyisoquinoline-6-carboxamide (5)

Compound 5 (yellow solid, 305 mg, yield 97%) was synthesized in the same manner as compound 3 in example 11, except compound 4 was used.

### Step 4: Synthesis of (S)-1-(3-hydroxypiperidin-1-yl)-7-methoxyisoquinoline-6-carboxamide (XXI)

Compound XXI (yellow solid, 200 mg, yield 92%) was synthesized in the same manner as compound XX in example 11, except compound 5 was used. LC-MS: [M+H]⁺ 302.1. ¹H NMR (400 MHz, DMSO-d₆) ¹H NMR (400 MHz, DMSO) δ 8.17 (s, 1H), 8.01 (d, J=5.6 Hz, 1H), 7.84 (s, 1H), 7.70 (s, 1H), 7.51 (s, 1H), 7.39 (d, J=5.7 Hz, 1H), 4.98 (d, J=4.7 Hz, 1H), 3.99 (s, 3H), 3.87 (dd, J=8.0, 3.9 Hz, 1H), 3.54 (d, J=9.7 Hz, 1H), 3.46 (d, J=12.7 Hz, 1H), 3.00 (t, J=10.1 Hz, 1H), 2.88 (dd, J=11.9, 8.3 Hz, 1H), 2.02-1.82 (m, 2H), 1.71 (m, 1H), 1.53-1.37 (m, 1H).

### Example 13

### Preparation of (S)-7-methoxy-1-(((5-oxopyrrolidin-2-yl) methyl) amino)-4-(prop-1-yn-1-yl) isoquinoline-6-carboxamide (XXII)

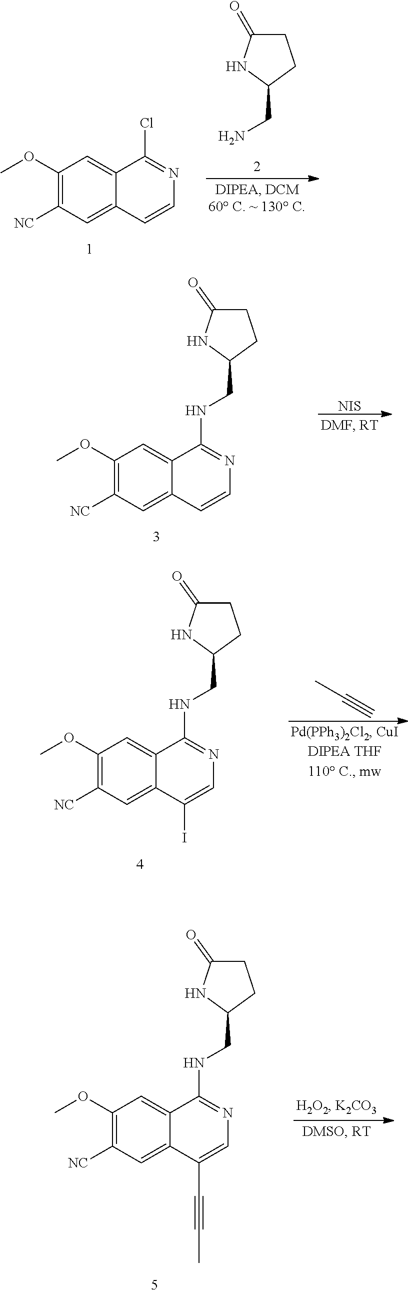

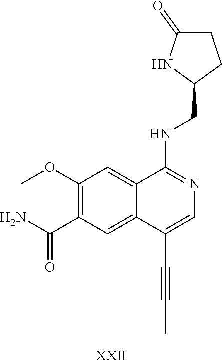

### Step 1: Synthesis of (S)-7-methoxy-1-(((5-oxopyrrolidin-2-yl) methyl) amino) isoquinoline-6-carbonitrile (3)

Compound 3 (yellow solid, 490 mg, yield 72%) was prepared in the same manner as compound 2 in example 11, except compound 2 was used.

### Step 2: Synthesis of (S)-4-iodo-7-methoxy-1-(((5-oxopyrrolidin-2-yl) methyl) amino) isoquinoline-6-carbonitrile (4)

To a solution of compound 3 (490 mg) in DMF (15 mL) was added NIS (409 mg) at rt. The resulting mixture was stirred at rt for 1 h. Title compound was obtained as a yellow solid (570 mg, yield 82%) after standard work up procedure.

### Step 3: Synthesis of (S)-7-methoxy-1-(((5-oxopyrrolidin-2-yl) methyl) amino)-4-(prop-1-yn-1-yl) isoquinoline-6-carbonitrile (5)

To a suspension of compound 4 (42 mg) in THF (2 mL) was added propyne (4 mL), Pd(PPh₃)₂Cl₂ (5 mg), CuI (5 mg), DIPEA (26 mg) at rt and the mixture was replaced with N₂ three times. The mixture was heated to 110° C. on microwave reactor for 2 h. Title compound was obtained as a yellow solid (16.7 mg, yield 50%) after standard work up procedure. LC-MS: [M+H]⁺ 335.1.

### Step 4: Synthesis of (S)-7-methoxy-1-(((5-oxopyrrolidin-2-yl) methyl) amino)-4-(prop-1-yn-1-yl) isoquinoline-6-carboxamide (XXII)

Compound XXII (yellow solid, 81 mg, yield 77%) was prepared in the same manner as compound XX, except compound 5 was used. LC-MS: [M+H]⁺ 353.2. ¹H NMR (400 MHz, DMSO-d₆) δ 8.32-3.31 (m, 1H), 7.99 (s, 1H), 7.90-7.88 (m, 3H), 7.80-7.77 (m, 2H), 4.1 (s, 3H), 3.86-3.78 (m, 1H), 3.62-3.55 (m, 2H), 2.31-2.12 (m, 2H), 2.1 (s, 3H), 2.01-1.81 (m, 2H).

### Example 14

### Preparation of 1-((1S, 3S) 3-aminocyclopentyl)-6-methoxy-1H-indole-5-carboxamide (XXIII)

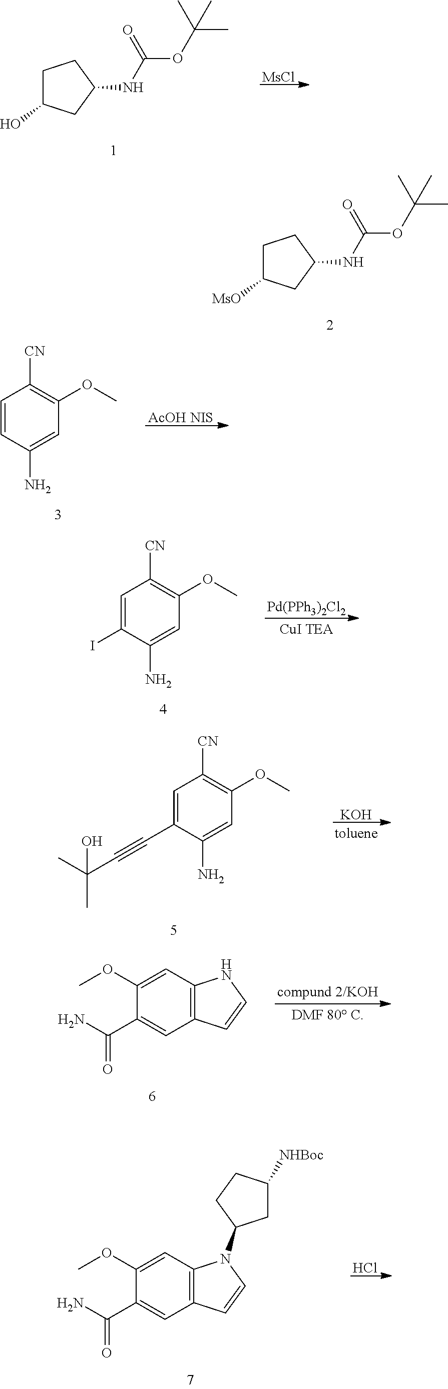

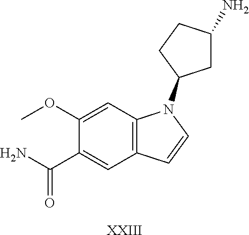

### Step 1: Synthesis of (1R, 3S)-3-((tert-butoxycarbonyl) amino) cyclopentyl methanesulfonate (2)

Compound 2 was prepared (white solid, 208 mg) in the same manner as compound 2 in example 3, except compound 1 and MsCl were used.

### Step 2: Synthesis of 4-amino-5-iodo-2-methoxybenzonitrile (4)

Compound 4 (2.02 g, yield 74%) was prepared in the same manner as compound 4 in example 14, except compound 3 and AcOH were used.

### Step 3: Synthesis of 4-amino-5-(3-hydroxy-3-methylbut-1-yn-1-yl)-2-methoxybenzonitrile (5)

Compound 5 was prepared (yellow solid, 1.03 g, yield 88%) in the same manner as compound 5 in example 14, except compound 4 and TEA were used.

### Step 4: Synthesis of 6-methoxy-1H-indole-5-carboxamide (6)

A mixture of compound 5 (1.03 g) and KOH (0.75 g) in toluene (20 mL) was stirred 12 h at 120° C. Then the mixture was concentrated and purified to give the title compound (361 mg, yield 47%).

### Step 5: Synthesis of tert-butyl ((1S, 3S)-3-(5-carbamoyl-6-methoxy-1H-indol-1-yl) cyclopentyl) carbamate (7)

A mixture of compound 6 (100 mg), compound 2 (180 mg) and KOH (140 mg) in DMF (5 ml) was stirred 1 h at 80° C. Then the mixture was concentrated and purified by prep-TLC to give the title compound 7 (66 mg, yield 35%).

### Step 6: Synthesis of 1-((1S, 3S)-3-aminocyclopentyl)-6-methoxy-1H-indole-5-carboxamide (XXIII)

The title compound XXIII (37 mg, yield 100%) was synthesized in the same manner as compound XX, except compound 7 was used. LC-MS: [M+H]⁺ 274.1.

### Example 15

### Preparation of (4²S,4³R)-4⁵-oxo-4³-propyl-2,6,9-trioxa-1 (1,7)-isoquinolina-4 (2,1)-pyrrolidinacyclononaphane-1⁶-carboxamide (XXIV)

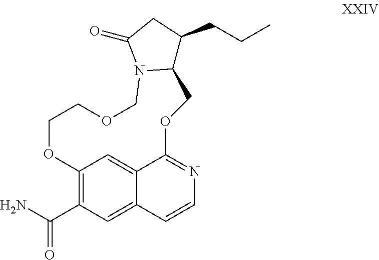 [SMILES: CCC[C@@H]1CC(=O)N2COCCOc3cc4c(nccc4cc3C(N)=O)OC[C@H]12]

Compound XXIV (25 mg, yield 52.9%) was synthesized in the same manner as compound XIX, except PrMgBr was used. LC-MS: [M+H]⁺ 400.3. ¹H NMR (400 MHz, CDCl₃) δ 8.68 (s, 1H), 8.62 (s, 1H), 8.01 (d, J=5.9 Hz, 1H), 7.82 (s, 1H), 7.41 (d, J=5.9 Hz, 1H), 5.92 (s, 1H), 4.98 (dd, J=14.2, 7.0 Hz, 1H), 4.88 (d, J=9.5 Hz, 1H), 4.71 (dd, J=10.8, 2.0 Hz, 1H), 4.63 (d, J=9.5 Hz, 1H), 4.59 (dd, J=14.2, 3.1 Hz, 1H), 4.41 (dd, J=10.8, 6.8 Hz, 1H), 4.06 (dd, J=12.7, 7.4 Hz, 1H), 3.99 (t, J=6.0 Hz, 1H), 3.92 (dd, J=13.4, 3.3 Hz, 1H), 2.66-2.57 (m, 1H), 2.51 (d, J=8.4 Hz, 2H), 1.41 (m, 4H), 0.99 (t, J=7.3 Hz, 3H).

### Example 16

### Preparation of (4²S, 4³R)-4³-methyl-4⁵-oxo-2, 6, 9-trioxa-1 (1,7)-isoquinolina-4 (2,1)-pyrrolidinacyclononaphane-1⁶-carboxamide (XXV)

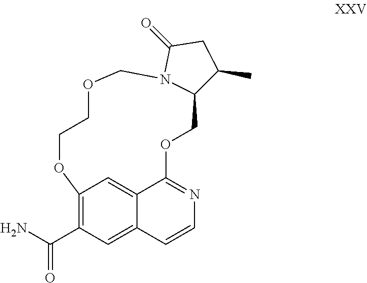 [SMILES: C[C@@H]1CC(=O)N2COCCOc3cc4c(nccc4cc3C(N)=O)OC[C@H]12]

Compound XXV (white solid, 73 mg, yield 67%) was synthesized in the same manner as compound XIX, except MeMgBr was used. LC-MS: [M+H]⁺ 372.1. ¹H NMR: (400 MHz, DMSO-d₆) δ 8.52 (s, 1H), 8.25 (s, 1H), 7.97 (d, J=5.8 Hz, 1H), 7.83 (s, 1H), 7.74 (s, 1H), 7.54 (d, J=5.8 Hz, 1H), 4.77 (dd, J=14.0, 7.1 Hz, 1H), 4.66 (td, J=5.9, 5.2, 2.0 Hz, 3H), 4.51 (dd, J=13.8, 3.9 Hz, 1H), 4.31 (dd, J=10.9, 7.4 Hz, 1H), 4.08-3.99 (m, 1H), 3.95 (dd, J=13.4, 7.1 Hz, 1H), 3.69 (dd, J=13.3, 4.0 Hz, 1H), 2.65 (p, J=7.2 Hz, 1H), 2.43 (dd, J=16.2, 8.0 Hz, 1H), 2.21 (dd, J=16.2, 7.9 Hz, 1H), 1.12 (d, J=7.0 Hz, 3H).

### Example 17

### 1-(4-(aminomethyl)-4-methylpiperidin-1-yl)-7-methoxyisoquinoline-6-carboxamide (XXVI)

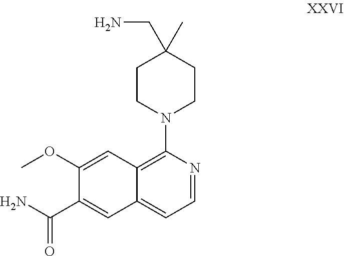 [SMILES: COc1cc2c(N3CCC(C)(CN)CC3)nccc2cc1C(N)=O]

Compound XXVI (yellow solid, 69 mg, yield 48%) was synthesized in the same manner as compound XX in example 11, except tert-butyl ((4-methylpiperidin-4-yl) methyl) carbamate was used. LC-MS: [M+H]⁺ 329.4. ¹H NMR (400 MHz, DMSO) δ 8.17 (s, 1H), 8.02 (d, J=5.6 Hz, 1H), 7.84 (s, 1H), 7.70 (s, 1H), 7.38 (d, J=5.8 Hz, 1H), 7.37 (s, 1H), 3.98 (s, 3H), 3.39 (m, 2H), 3.23-3.11 (m, 4H), 2.49 (s, 2H), 1.77-1.64 (m, 2H), 1.47 (d, J=14.6 Hz, 2H), 0.97 (s, 3H).

### Example 18

### Preparation of 1-(4-aminopiperidin-1-yl)-7-methoxyisoquinoline-6-carboxamide (XXVII)

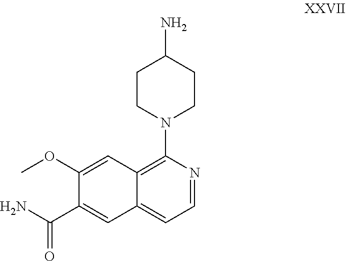 [SMILES: COc1cc2c(N3CCC(N)CC3)nccc2cc1C(N)=O]

Compound XXVII (10 mg, yield 34%) was synthesized in the same manner as compound XX in example 11, except benzyl piperidin-4-ylcarbamate was used. LC-MS: [M+H]⁺ 301.4.

### Example 19

### Preparation of 1-((((2S, 3R)-3-ethyl-5-oxopyrrolidin-2-yl) methyl) amino)-7-methoxyisoquinoline-6-carboxamide (XXVIII)

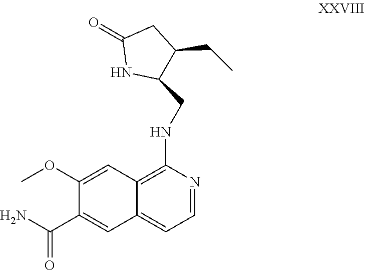 [SMILES: CC[C@@H]1CC(=O)N[C@@H]1CNc1nccc2cc(C(N)=O)c(OC)cc12]

Compound XXVIII (white solid, 33 mg, yield 52%) was prepared in the sample manner as compound XX in example 11, except compound (4R,5S)-5-(aminomethyl)-4-ethylpyrrolidin-2-one was used. LC-MS: [M+H]⁺ 343.1. ¹H NMR: (400 MHz, DMSO-d₆) δ 8.03 (s, 1H), 7.96 (s, 1H), 7.84-7.76 (m, 2H), 7.74 (s, 1H), 7.69-7.63 (m, 1H), 7.38 (t, J=5.3 Hz, 1H), 6.93 (d, J=5.8 Hz, 1H), 4.00 (s, 3H), 3.97-3.88 (m, 1H), 3.83 (ddd, J=13.1, 5.8, 4.0 Hz, 1H), 2.39 (q, J=7.9 Hz, 1H), 2.20 (dd, J=16.2, 8.2 Hz, 1H), 2.08-1.97 (m, 1H), 1.65-1.58 (m, 2H), 1.43 (ddd, J=13.7, 9.1, 7.2 Hz, 1H), 0.95 (t, J=7.3 Hz, 3H).

### Example 20

### Preparation of (R)-7-methoxy-1-(piperidin-3-ylamino) isoquinoline-6-carboxamide (XXIX)

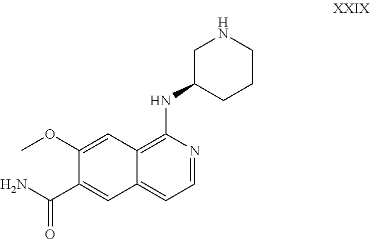 [SMILES: COc1cc2c(N[C@@H]3CCCNC3)nccc2cc1C(N)=O]

Compound XXIX (76 mg, yield 66%) was synthesized in the same manner as compound XX in example 11, except tert-butyl (R)-3-aminopiperidine-1-carboxylate was used.

LC-MS: [M+H]⁺ 301.4. ¹H NMR (400 MHz, DMSO-d₆) δ 9.82 (s, 1H), 8.92 (m, 1H), 8.70 (s, 1H), 8.24-8.19 (d, J=20.0 Hz, 1H), 7.92-7.82 (d, J=40 Hz, 2H), 7.62 (s, 1H), 7.22 (s, 1H), 4.53 (s, 1H), 4.07 (s, 3H), 3.21-3.17 (m, 2H), 3.15-3.00 (m, 2H), 2.00-1.91 (m, 2H), 1.90-1.82 (m, 2H).

### Example 21

### Preparation of (R)-1-((1-(2-cyanoacetyl) piperidin-3-yl) amino)-7-methoxyisoquinoline-6-carboxamide (XXX)

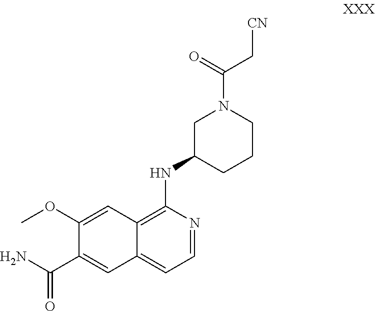 [SMILES: COc1cc2c(N[C@@H]3CCCN(C(=O)CC#N)C3)nccc2cc1C(N)=O]

Compound XXX (3 mg, yield 12%) was synthesized in the same manner as compound XXXI in example 22, except compound XXIX was used. LC-MS: [M+H]⁺ 368.3. ¹H NMR (400 MHz, DMSO-d₆) δ 8.04 (d, J=5.9 Hz, 1H), 7.87-7.76 (m, 2H), 7.68 (d, J=12.8 Hz, 2H), 7.08 (d, J=38.6 Hz, 1H), 6.98 (dd, J=12.8, 5.7 Hz, 1H), 4.41 (dd, J=129.1, 11.1 Hz, 1H), 4.11 (dd, J=31.5, 12.8 Hz, 3H), 3.99 (s, 3H), 3.66 (d, J=12.1 Hz, 1H), 2.98 (dd, J=20.6, 10.2 Hz, 2H), 2.82-2.55 (m, 1H), 2.10 (s, 1H), 1.90-1.40 (m, 2H).

### Example 22

### Preparation of (R)-1-((1-(2-cyanoacetyl) piperidin-3-yl) thio)-7-methoxyisoquinoline-6-carboxamide (XXXI)

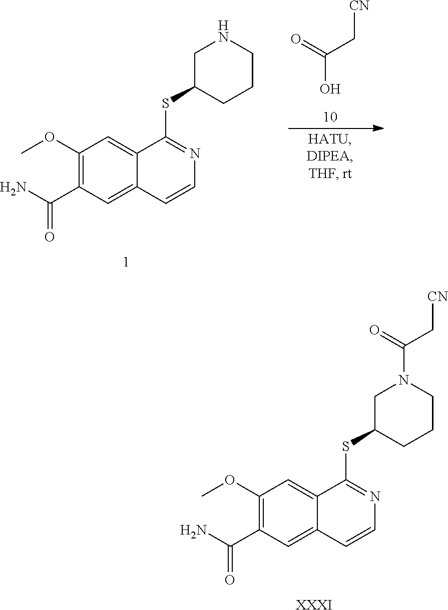

To a suspension of compound 1 (190 mg, synthesized in the same manner as compound XX in example 11, except tert-butyl (R)-3-mercaptopiperidine-1-carboxylate was used) in DCM (10 mL) was added compound 10 (61 mg) and HATU (341 mg) at rt. And then, DIPEA (232 mg) was added dropwise into the mixture at rt. The reaction was stirred overnight at rt. The title compound was obtained (85 mg, yield 37%) after standard work up procedure. LC-MS [M+H]⁺: 385.3. ¹H NMR (400 MHz, DMSO-d₆) δ 8.30 (dd, J=18.2, 5.6 Hz, 1H), 8.23 (d, J=3.6 Hz, 1H), 7.88 (s, 1H), 7.76 (s, 1H), 7.64 (dd, J=9.5, 5.7 Hz, 1H), 7.34 (s, 1H), 4.34-4.15 (m, 1H), 4.13-4.03 (m, 3H), 4.00 (d, J=1.5 Hz, 3H), 3.58-3.07 (m, 3H), 2.23-2.13 (m, 1H), 1.89-1.58 (m, 3H).

### Example 23

### Preparation of (R)-7-methoxy-1-(pyrrolidin-3-ylamino)isoquinoline-6-carboxamide (XXXII)

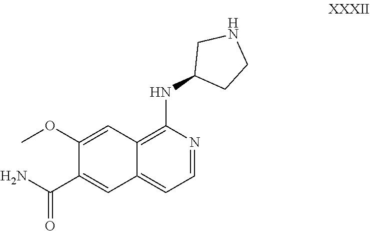 [SMILES: COc1cc2c(N[C@@H]3CCNC3)nccc2cc1C(N)=O]

Compound XXXII (25 mg, yield 36%) was synthesized in the same manner as compound XX in example 11, except tert-butyl (R)-3-aminopyrrolidine-1-carboxylate was used. LC-MS [M+H]⁺: 287.1

### Example 24

### Preparation of (R)-7-methoxy-1-(pyrrolidin-3-ylamino)isoquinoline-6-carboxamide (XXXIII)

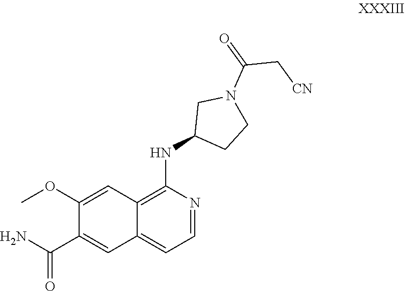 [SMILES: COc1cc2c(N[C@@H]3CCN(C(=O)CC#N)C3)nccc2cc1C(N)=O]

Compound XXXIII (69 mg, yield 17%) was synthesized in the same manner as compound XXXI in example 22, except compound XXXII was used. LC-MS: [M+H]⁺ 354.1. ¹H NMR (400 MHz, DMSO-d₆) δ 8.05 (d, J=1.6 Hz, 1H), 8.73-8.71 (m, 2H), 7.70-7.66 (m, 2H), 7.31-7.29 (m, 1H), 7.02-7.00 (m, 1H), 4.77-4.62 (m, 1H), 3.99 (s, 3H), 3.68-3.61 (m, 2H), 3.59-3.40 (m, 3H), 3.17-3.12 (m, 1H), 2.34-2.23 (m, 1H), 2.14-2.05 (m, 1H).

### Example 25

### Preparation of Preparation of preparation of (S)-1-(3-aminopiperidin-1-yl)-7-methoxy-4-(prop-1-yn-1-yl)soquinoline-6-carboxamide (XXXIV)

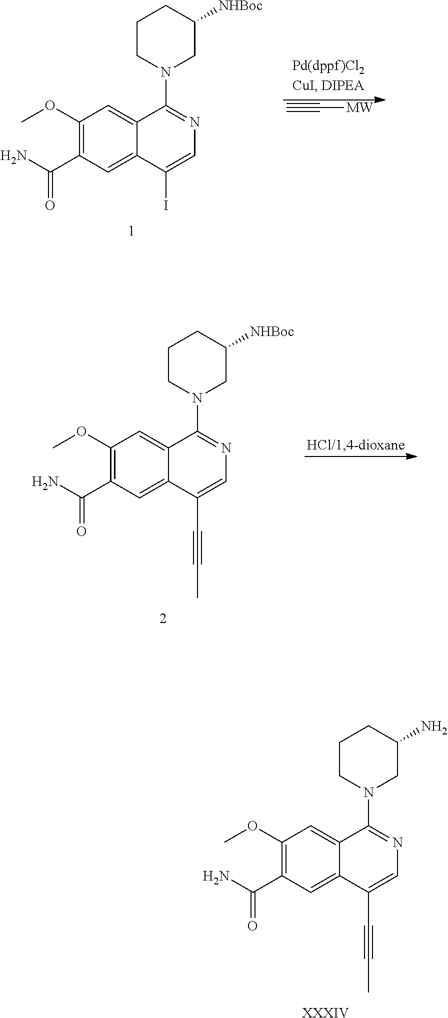

### Step 1: Preparation of tert-butyl (S)-(1-(6-carbamoyl-7-methoxy-4-(prop-1-yn-1-yl)isoquinolin-1-yl)piperidin-3-yl)carbamate

The title compound (110 mg, yield 90%) was synthesized in the same manner as compound 5 in example 13, except compound 1 was used. LC-MS: [M+H]⁺ 439.4 Step 2: Preparation of (S)-1-(3-aminopiperidin-1-yl)-7-methoxy-4-(prop-1-yn-1-yl) soquinoline-6-carboxamide (XXXIV)

The title compound (15 mg, yield 66%) was synthesized in the same manner as compound XX in example 11, except compound 2 was used. LC-MS: [M+H]⁺ 339.4. ¹H NMR (400 MHz, DMSO-d₆) δ 8.37 (s, 4H), 8.20 (s, 1H), 8.11 (s, 1H), 7.53-7.39 (m, 1H), 4.07 (s, 1H), 4.03 (s, 3H), 3.73 (d, J=13.1 Hz, 2H), 3.27-3.13 (m, 2H), 2.20 (d, J=3.2 Hz, 3H), 2.04 (s, 2H), 1.74 (dd, J=19.9, 10.6 Hz, 2H).

### Example 26

### Preparation of(S)-1-(3-aminopiperidin-1-yl)-4-bromo-7-methoxyisoquinoline-6-carboxamide (XXXV)

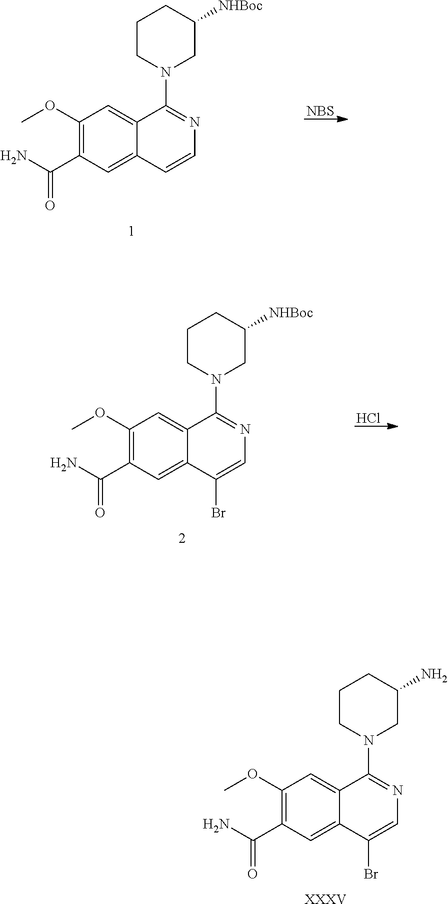

### Step 1: Preparation of tert-butyl (S)-(1-(4-bromo-6-carbamoyl-7-methoxyisoquinolin-1-yl)piperidin-3-yl)carbamate

Compound 2 (60.3 mg, yield 100%) was synthesized in the same manner as compound 4 in example 13, except NBS was used.

### Step 2: preparation of (S)-1-(3-aminopiperidin-1-yl)-4-bromo-7-methoxyisoquinoline-6-carboxamide (XXXV)

The title compound (44 mg, yield 100%) was synthesized in the same manner as compound XX in example 11, except compound 2 was used. LC-MS: [M+H]⁺ 379.2, ¹H NMR (400 MHz, D₂O) δ 7.99 (s, 1H), 7.86 (s, 1H), 7.17 (s, 1H), 3.95 (s, 3H), 3.75 (dd, J=13.9, 7.0 Hz, 1H), 3.64 (dd, J=15.8, 12.8 Hz, 2H), 3.48 (d, J=13.9 Hz, 1H), 3.12 (t, J=10.6 Hz, 1H), 2.16 (s, 1H), 2.00 (d, J=12.5 Hz, 1H), 1.88 (d, J=9.5 Hz, 1H), 1.72 (d, J=9.7 Hz, 1H).

### Example 27

### Preparation of (S)-1-(3-aminopiperidin-1-yl)-4-cyano-7-methoxyisoquinoline-6-carboxamide (XXXVI)

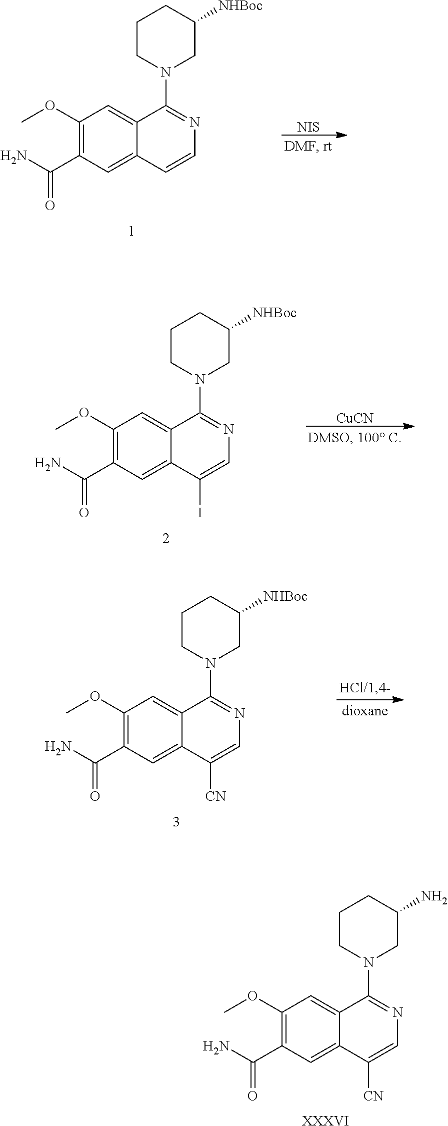

### Step 1: Preparation of tert-butyl (S)-(1-(6-carbamoyl-4-iodo-7-methoxyisoquinolin-1-yl) piperidin-3-yl) carbamate (2)

Compound 2 (130 mg, yield 49%) was synthesized in the same manner as compound 4 in example 13 except compound 1 was used.

### Step 2: Preparation of tert-butyl (S)-(1-(6-carbamoyl-4-cyano-7-methoxyisoquinolin-1-yl) piperidin-3-yl) carbamate (3)

To a solution of compound 2 (130 mg) in DMSO (5 mL) was added CuCN (46 mg) at rt. The mixture was stirred at 120° C. for 3 h. The title compound 3 was obtained as a yellow solid (65 mg, yield: 62%) after standard work up procedure. LC-MS: [M+H]⁺ 426.3

### Step 3: Preparation of (S)-1-(3-aminopiperidin-1-yl)-4-cyano-7-methoxyisoquinoline-6-carboxamide (XXXVI)

The title compound (15 mg, yield: 66%) was synthesized in the same manner as compound XX in example 11, except compound 3 was used. LC-MS: [M+H]⁺ 326.3. ¹H NMR (400 MHz, DMSO-d₆) δ 8.59 (d, J=2.9 Hz, 1H), 8.32 (s, 2H), 8.22 (s, 1H), 7.95 (d, J=30.0 Hz, 2H), 7.52 (s, 1H), 4.13 (m, 1H), 4.09 (s, 3H), 4.03 (m, 2H), 3.72 (d, J=12.9 Hz, 2H), 2.20-1.94 (m, 2H), 1.82-1.59 (m, 2H).

### Assays

Protocols that may be used to determine the recited potency for the compounds of the disclosure are described below.

The kinase activity of IRAK4 is measured by its ability to phosphorylate a fluorescently labeled synthetic peptide in the presence of ATP. The assay format is based on the Immobilized Metal Ion Affinity-Based Fluorescence Polarization (IMAP) platform developed by Molecular Devices. Briefly, reaction mixture (20 μL) contains the assay buffer (20 mM Tris.Cl, pH 7.2, 1 mM MgCl₂, 1 mM DTT, and 0.02% Tween 20), 0.5 nM GST tagged IRAK4 (SignalChem), 100 nM peptide substrate and 100 μM ATP. The amino acid sequence of the peptide substrate is 5FAM-RKRQGSVRRRVH-COOH (Cat #: RP7030, Molecular Devices). The reaction is initiated by adding substrates ATP and RP7030, and terminated by adding Stop solution (60 μL) after 30 minutes of incubation at 25° C. The Stop solution is prepared with IMAP Progressive Reagent A/B and Binding reagent according to vender's instruction. The extent of phosphorylation of the peptide is measured by changes in Fluorescence Polarization (FP) resulting from binding of phosphate group on the peptide with immobilized metal coordination complexes on the nanoparticles included in the Stop solution. Errors in the calculated IRAK4 IC₅₀ values range from 4-12% from duplicate experiments.

The testing results for representative compounds are summarized in Table II, wherein + represents the IC₅₀ value of <0.5 M; ++ represents the IC₅₀ value of 0.5-3 μM; and +++ represents the IC₅₀ value of 3-10 M.

TABLE II
IRAK4 Inhibitory Activity of Representative Examples

| Compound | IRAK4 |
|---|---|
| No | IC₅₀ |
| II | +++ |
| IV | +++ |
| VII | ++ |
| IX | +++ |
| XI | ++ |
| XIII | ++ |
| XIV | ++ |
| XVII | ++ |
| XVIII | + |
| XIX | ++ |
| XX | + |
| XXI | ++ |
| XXII | + |
| XXIII | ++ |
| XXIV | ++ |
| XXV | + |
| XXVI | ++ |
| XXVII | ++ |
| XXVIII | + |
| XXIX | ++ |
| XXX | +++ |
| XXXI | +++ |
| XXXII | ++ |
| XXXIII | ++ |
| XXXIV | + |
| XXXV | ++ |
| XXXVI | ++ |

Unless otherwise indicated, all numbers expressing quantities of ingredients, properties such as molecular weight, reaction conditions, and etc. used in herein are to be understood as being modified in all instances by the term “about.” Each numerical parameter should at least be construed in light of the number of reported significant digits and by applying ordinary rounding techniques. Accordingly, unless indicated to the contrary, the numerical parameters may be modified according to the desired properties sought to be achieved, and should, therefore, be considered as part of the disclosure. At the very least, the examples shown herein are for illustration only, not as an attempt to limit the scope of the disclosure.

The terms “a,” “an,” “the” and similar referents used in the context of describing embodiments of the present disclosure (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context.

The use of any and all examples, or exemplary language (e.g., “such as”) provided herein is intended merely to better illustrate embodiments of the present disclosure and does not pose a limitation on the scope of any claim. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the embodiments. Of course, variations on these described embodiments will become apparent to those of ordinary skill in the art upon reading the foregoing description. The inventor expects skilled artisans to employ such variations as appropriate, and the inventors intend for the embodiments of the present disclosure to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A compound represented by a formula: 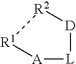
or a pharmaceutically acceptable salt thereof;
wherein a dashed line indicates the presence or absence of a covalent bond;
A is an optionally substituted fused bicyclic heteroaryl group, an optionally substituted naphthyl group, or an optionally substituted fused tricyclic heteroaryl group, wherein A contains a R¹ substituent;
L is a direct covalent bond, optionally substituted C₁₋₃H₂₋₆X₀₋₁, or X, wherein X is O, S, SO, SO₂, or NH;
D is an optionally substituted heterocyclic ring, or an optionally substituted fused or spiro bicyclic group;
R¹ is H, —NR^{A}R^{B}, —OR^{A}, —O—R^{A}—O—R^{B}, —O—R^{A}—O—R^{B}—O—R^{C}, —C(O)NR^{A}R^{B}, or —SR^{A};
R² is H, —C(O)— or a direct covalent bond to R¹; and
R^{A}, R^{B}, R^{C}, and R^{D} are independently H or C₁₋₁₂ hydrocarbyl.

2. The compound of claim 1, wherein R¹ and L attach to A such that 4 ring atoms of A directly connect R¹ to L.

3. The compound of claim 1, wherein A-L is A-L is A-S(O)₀₋₂C(R^{A})(R^{B})—, A-OC(R^{A})(R^{B})—, A-N(R^{C})C(R^{A})(R^{B})—, A-S(O)₀₋₂C(R^{A})(R^{B})—, 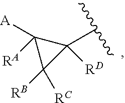
or A-C(R^{A})(R^{B})C(R^{C})(R^{D})—, A-N(R^{C})—, A-S(O)₀₋₂, or L is a direct covalent bond.

4. The compound of claim 1, 2, or 3, wherein A is optionally substituted 2-oxo-2,3-dihydro-1H-imidazo[4,5-g]isoquinolin-4-yl.

5. The compound of claim 1, 2, or 3, wherein A is optionally substituted isoquinolinyl.

6. The compound of claim 1, 2, or 3, wherein A is optionally substituted 1,3-dioxo-2,3-dihydro-1H-pyrrolo[3,4-g]isoquinolinyl

7. The compound of claim 1, 2, or 3, wherein A is optionally substituted 3-imino-2-oxo-2,3-dihydro-1H-pyrrolo[2,3-g]isoquinolinyl, or optionally substituted 3-(hydroxyimino)-2-oxo-2,3-dihydro-1H-pyrrolo[2,3-g]isoquinolin-yl.

8. The compound of claim 1, 2, or 3, wherein A is optionally substituted indolyl.

9. The compound of claim 1, 2, or 3, wherein A is optionally substituted benzoimidazolyl.

10. The compound of claim 1, 2, or 3, wherein A is optionally substituted 1H-imidazo[1,2-a]indolyl.

11. The compound of claim 1, 2, or 3, wherein A is optionally substituted naphtho[2,3-b]thiophenyl.

12. The compound of claim 1, 2, or 3, wherein A is optionally substituted thiazolo[3,2 a]indolyl.

13. The compound of claim 1, 2, or 3, wherein A is optionally substituted 1-H-benzo[f]indolyl.

14. The compound of claim 1, 2, or 3, wherein A is optionally substituted 2-oxo-1,2-dihydrobenzo[g]quinoxalinyl.

15. The compound of claim 1, 2, or 3, wherein A is optionally substituted 2-oxo-1,2,3,4-tetrahydrobenzo[g]quinoxalinyl.

16. The compound of claim 1 or 2, wherein A is optionally substituted naphtho[2,3-b]furanyl.

17. The compound of claim 1 or 2, wherein A is optionally substituted oxazolo[3,2-a]indolyl.

18. The compound of claim 1, 2, or 3, wherein A is optionally substituted 3H-imidazo[4,5-c]isoquinolin-2-amine.

19. The compound of claim 1, 2, or 3, wherein A is optionally substituted thiazolo[5,4-c]isoquinolin-2-amine.

20. The compound of claim 1, 2, or 3, wherein A is optionally substituted oxazolo[5,4-c]isoquinolin-2-amine.

21. The compound of claim 1, 2, or 3, wherein A is optionally substituted quinolone.

22. The compound of claim 1, 2, or 3, wherein A is optionally substituted 6-carbamoyl-7-methoxyisoquinolin-1-yl.

23. The compound of claim 1, 2, or 3, wherein A is 6-carbamoyl-7-methoxyisoquinolin-1-yl.

24. The compound of claim 1, 2, or 3, wherein A is optionally substituted 6-carbamoyl-7-methoxy-4-(prop-1-yn-1-yl)isoquinolin-1-yl.

25. The compound of claim 1, 2, or 3, wherein A is 6-carbamoyl-7-methoxy-4-(prop-1-yn-1-yl)isoquinolin-1-yl.

26. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —OC(O)NH₂ substituent.

27. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C(O)NH₂ substituent.

28. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C(O)NHOH substituent.

29. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C(O)NHS(O)₂CH₃ substituent.

30. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C(O)NHCN substituent.

31. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —OH substituent.

32. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C(O)CHF₂ substituent.

33. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —NHC(O)CH₃ substituent.

34. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —NH₂ substituent.

35. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C(S)NH₂ substituent.

36. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —SC(O)NH₂ substituent.

37. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —OC(S)NH₂ substituent.

38. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —NHC(S)NH₂ substituent.

39. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C(O)SH substituent.

40. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —NHC(═NCH₃)NH₂ substituent.

41. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —NHC(O)SCH₃ substituent.

42. The compound of claim 1, 2, 3, 4, 5, 6, 7, 8, 9, 10, 11, 12, 13, 14, 15, 16, 17, 18, 19, 20, 21, 22, 23, 24, or 25, wherein A has an —NHC(O)OCH₃ substituent.

43. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —C≡C—CH₃ substituent.

44. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —Br substituent.

45. The compound of claim 1, 2, 3, 4, 5, 6, 7, 8, 9, 10, 11, 12, 13, 14, 15, 16, 17, 18, 19, 20, 21, 22, 23, 24, or 25, wherein A has a —CN substituent.

46. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 5-oxopyrrolidinyl.

47. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 2-oxooxazolidinyl.

48. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 2-oxoimidazolidinyl.

49. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted octahydrocyclopenta[c]pyrrolyl.

50. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted azetidinyl.

51. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 4-oxohexahydropyrrolo[3,4-c]pyrrol-2(1H)-yl.

52. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted piperidine.

53. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted cyclopentane.

54. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted piperazine.

55. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 1H-1,2,3-triazole.

56. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 2-oxa-8-azaspiro[4.5]decane.

57. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted pyrrolidine.

58. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 5-oxopyrrolidin-2-yl.

59. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 3-ethyl-5-oxopyrrolidin-2-yl.

60. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is 3-ethyl-5-oxopyrrolidin-2-yl.

61. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is optionally substituted 3-aminopiperidin-1-yl.

62. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D is 3-aminopiperidin-1-yl.

63. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D has an —NH₂ substituent.

64. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D has an —OH substituent.

65. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D has a —CH₃ substituent.

66. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D has a —CH₂CH₃ substituent.

67. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D has a —CH₂CH₂CH₃ substituent.

68. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D has a —CH₂NH₂ substituent.

69. The compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45, wherein D has both —CH₃ and —CH₂NH₂ substituents on the same ring C-atom.

70. The compound of any preceding claim, wherein R^{A} is H.

71. The compound of any preceding claim, wherein R^{B} is H.

72. The compound of any preceding claim, wherein R^{C} is H.

73. The compound of any preceding claim, wherein R^{D} is H.

74. The compound of any preceding claim, wherein R¹ is H.

75. The compound of any preceding claim, wherein R¹ is —OCH₃.

76. The compound of any preceding claim, wherein R¹ is —NHCH₃.

77. The compound of any preceding claim, wherein R¹ is —NH₂.

78. The compound of any preceding claim, wherein L is —O—CH₂—.

79. The compound of any preceding claim, wherein L is —NH—CH₂—.

80. The compound of any preceding claim, wherein L is a bond.

81. The compound of any preceding claim, wherein L is a bond, and the N ring atom of the ring D is directly connected to the ring A.

82. The compound of any preceding claim, wherein R² is H.

83. The compound of any preceding claim, wherein R² is —C(O)—.

84. The compound of any preceding claim, wherein R² is a direct covalent bond to R¹.

85. The compound of any preceding claim, wherein there is a covalent bond between R¹ and R², and R¹—R² is —OCH₂CH₂OCH₂—.

86. The compound of any preceding claim, wherein there is a covalent bond between R¹ and R², and R¹—R² is —OCH₂CH₂OCH₂CH₂—.

87. The compound of any preceding claim, wherein there is a covalent bond between R¹ and R², and R¹—R² is —OCH₂CH₂OCH₂CH₂OCH₂—.

88. The compound of any preceding claim, wherein there is a covalent bond between R¹ and R², and R¹—R² is —OCH₂CH₂OCH₂C(O)—.

89. A compound, or a pharmaceutically acceptable salt thereof, wherein the compound is: optionally substituted 5-((5-oxopyrrolidin-2-yl)methoxy)-1,3-dihydro-2H-imidazo[4,5-g]isoquinolin-2-one, optionally substituted 7-methoxy-1-((5-oxopyrrolidin-2-yl)methoxy)isoquinolin-6-yl carbamate, optionally substituted 7-(methylamino)-1-((5-oxopyrrolidin-2-yl)methoxy)isoquinoline-6-carboxamide, optionally substituted N-hydroxy-7-methoxy-1-((5-oxopyrrolidin-2-yl)methoxy)isoquinoline-6-carboxamide, optionally substituted 7-methoxy-1-(((5-oxopyrrolidin-2-yl)methyl)amino)isoquinoline-6-carboxamide, optionally substituted 5-((5-oxopyrrolidin-2-yl)methoxy)-1H-pyrrolo[3,4-g]isoquinoline-1,3(2H)-dione, optionally substituted (E)-3-(hydroxyimino)-5-((5-oxopyrrolidin-2-yl)methoxy)-1,3-dihydro-2H-pyrrolo[2,3-g]isoquinolin-2-one, optionally substituted 5-(((6-(2,2-difluoroacetyl)-7-methoxyisoquinolin-1-yl)oxy)methyl)pyrrolidin-2-one, optionally substituted 4⁵-oxo-2,6,9-trioxa-1(1,7)-isoquinolina-4(2,3)-pyrrolidinacyclononaphane-1⁶-carboxamide, optionally substituted 4⁵-oxo-2,6,9-trioxa-1(1,7)-isoquinolina-4(2,1)-pyrrolidinacyclononaphane-1⁶-carboxamide, optionally substituted 4⁵-oxo-2,7,10-trioxa-1(1,7)-isoquinolina-4(2,3)-pyrrolidinacyclodecaphane-1⁶-carboxamide, optionally substituted 4⁵-oxo-2,7,10-trioxa-1(1,7)-isoquinolina-4(2,1)-pyrrolidinacyclodecaphane-1⁶-carboxamide, optionally substituted 1²-oxo-3,5,8-trioxa-1(4,3)-oxazolidina-4(1,7)-isoquinolinacyclononaphane-4⁶-carboxamide, optionally substituted 4²-oxo-2,6,9-trioxa-1(1,7)-isoquinolina-4(4,3)-imidazolidinacyclononaphane-1⁶-carboxamide, optionally substituted (4^{3a}R,4^{6a}R)-5-oxo-4¹,4²,4³,4^{3a}, 4⁴,4⁵, 4⁶,4^{6a}-octahydro-2,7,10-trioxa-1(1,7)-isoquinolina-4(6,2)-cyclopenta[c]pyrrolacyclodecaphane-1⁶-carboxamide, optionally substituted 5-oxo-2,7,10-trioxa-1(1,7)-isoquinolina-4(3,1)-azetidinacyclodecaphane-1⁶-carboxamide, optionally substituted 6-methoxy-1-(2-(5-oxopyrrolidin-2-yl)ethyl)-1H-indole-5-carboxamide, optionally substituted (4^{3a}R,4^{6a}S)-4³,5-dioxo-4¹,4²,4³,4^{3a},4⁴,4⁵,4⁶,4^{6a}-octahydro-2,7,10-trioxa-1(1,7)-isoquinolina-4(1,5)-pyrrolo[3,4-c]pyrrolacyclodecaphane-1⁶-carboxamide, optionally substituted 7-methoxy-1-(2-(5-oxopyrrolidin-2-yl)cyclopropyl)isoquinoline-6-carboxamide, optionally substituted 7-methoxy-9-((5-oxopyrrolidin-2-yl)methoxy)-1H-imidazo[1,2-a]indole-6-carboxamide, optionally substituted 6-methoxy-1-(2-(5-oxopyrrolidin-2-yl)ethyl)-1H-benzo[d]imidazole-5-carboxamide, optionally substituted 7-methoxy-9-((5-oxopyrrolidin-2-yl)methoxy)naphtho[2,3-b]thiophene-6-carboxamide, optionally substituted 7-methoxy-9-((5-oxopyrrolidin-2-yl)methoxy)thiazolo[3,2-a]indole-6-carboxamide, optionally substituted 7-methoxy-1-methyl-9-((5-oxopyrrolidin-2-yl)methoxy)-1H-benzo[f]indole-6-carboxamide, optionally substituted 3-methoxy-5-(((5-oxopyrrolidin-2-yl)methyl)sulfonyl)-2-naphthamide, optionally substituted 5-methoxy-3-((5-oxopyrrolidin-2-yl)methoxy)-1H-indole-6-carboxamide, optionally substituted 3-amino-6-((5-oxopyrrolidin-2-yl)methoxy)benzo[g]quinoxalin-2(1H)-one, optionally substituted 7-methoxy-9-((5-oxopyrrolidin-2-yl)methoxy)naphtho[2,3-b]furan-6-carboxamide, optionally substituted 6-((5-oxopyrrolidin-2-yl)methoxy)-3,4-dihydrobenzo[g]quinoxalin-2(1H)-one, optionally substituted 7-methoxy-9-((5-oxopyrrolidin-2-yl)methoxy)oxazolo[3,2-a]indole-6-carboxamide, optionally substituted 6-((5-oxopyrrolidin-2-yl)methoxy)benzo[g]quinoxalin-2(1H)-one, optionally substituted (S)-7-methoxy-1-((5-oxopyrrolidin-2-yl)methoxy)isoquinoline-6-carbothioamide, optionally substituted (S)—S-(7-methoxy-1-((5-oxopyrrolidin-2-yl)methoxy)isoquinolin-6-yl) carbamothioate, optionally substituted (S)—O-(7-methoxy-1-((5-oxopyrrolidin-2-yl)methoxy)isoquinolin-6-yl) carbamothioate, optionally substituted (S)-1-(7-methoxy-1-((5-oxopyrrolidin-2-yl)methoxy)isoquinolin-6-yl)thiourea, optionally substituted (S)-7-methoxy-1-((5-oxopyrrolidin-2-yl)methoxy)isoquinoline-6-carbothioic S-acid, optionally substituted (S,E)-1-(7-methoxy-1-((5-oxopyrrolidin-2-yl)methoxy)isoquinolin-6-yl)-2-methylguanidine, optionally substituted S-methyl (S)-(7-methoxy-1-((5-oxopyrrolidin-2-yl)methoxy)isoquinolin-6-yl)carbamothioate, optionally substituted methyl (S)-(7-methoxy-1-((5-oxopyrrolidin-2-yl)methoxy)isoquinolin-6-yl)carbamate, optionally substituted (S)-1-(3-aminopiperidin-1-yl)-7-methoxyisoquinoline-6-carboxamide, optionally substituted (S)-1-(3-hydroxypiperidin-1-yl)-7-methoxyisoquinoline-6-carboxamide, optionally substituted 1-(4-aminopiperidin-1-yl)-7-methoxyisoquinoline-6-carboxamide, optionally substituted 1-((1S,3S)-3-aminocyclopentyl)-6-methoxy-1H-indole-5-carboxamide, optionally substituted (S)-7-methoxy-1-(((5-oxopyrrolidin-2-yl)methyl)amino)-4-(prop-1-yn-1-yl)isoquinoline-6-carboxamide, optionally substituted (4²S,4³R)-4³-ethyl-4⁵-oxo-2,6,9-trioxa-1(1,7)-isoquinolina-4(2,1)-pyrrolidinacyclononaphane-1⁶-carboxamide, optionally substituted (4²S,4³R)-4³-methyl-4⁵-oxo-2,6,9-trioxa-1(1,7)-isoquinolina-4(2,1)-pyrrolidinacyclononaphane-1⁶-carboxamide, optionally substituted (4²S,4³R)-4⁵-oxo-4³-propyl-2,6,9-trioxa-1(1,7)-isoquinolina-4(2,1)-pyrrolidinacyclononaphane-1⁶-carboxamide, optionally substituted 1-(4-(aminomethyl)-4-methylpiperidin-1-yl)-7-methoxyisoquinoline-6-carboxamide, optionally substituted 1-((3R,4S)-3-amino-4-ethylpyrrolidin-1-yl)-7-methoxyisoquinoline-6-carboxamide, optionally substituted 1-((3R,5S)-3-amino-5-hydroxypiperidin-1-yl)-7-methoxyisoquinoline-6-carboxamide, optionally substituted (S)-1-(3-aminopiperazin-1-yl)-7-methoxyisoquinoline-6-carboxamide, optionally substituted (4²S,4³R)-4³-methyl-4⁵-oxo-1⁴-(prop-1-yn-1-yl)-2,6,9-trioxa-1(1,7)-isoquinolina-4(2,1)-pyrrolidinacyclononaphane-1⁶-carboxamide, optionally substituted 41H-2,6,9-trioxa-1(1,7)-isoquinolina-4(5,1)-triazolacyclononaphane-1⁶-carboxamide, optionally substituted (S)-2-amino-7-methoxy-5-(((5-oxopyrrolidin-2-yl)methyl)amino)-3H-imidazo[4,5-c]isoquinoline-8-carboxamide, optionally substituted (S)-2-amino-7-methoxy-5-(((5-oxopyrrolidin-2-yl)methyl)amino)thiazolo[5,4-c]isoquinoline-8-carboxamide, optionally substituted (S)-2-amino-7-methoxy-5-(((5-oxopyrrolidin-2-yl)methyl)amino)oxazolo[5,4-c]isoquinoline-8-carboxamide, optionally substituted 1-((((2S,3R)-3-ethyl-5-oxopyrrolidin-2-yl)methyl)amino)-7-methoxyisoquinoline-6-carboxamide, optionally substituted (4²S,4³S,4⁴S)-4⁴-fluoro-4³-methyl-4⁵-oxo-2,6,9-trioxa-1(1,7)-isoquinolina-4(2,1)-pyrrolidinacyclononaphane-1⁶-carboxamide, optionally substituted (4²S,4³,4⁴S)-4³-ethyl-4⁴-fluoro-4⁵-oxo-2,6,9-trioxa-1(1,7)-isoquinolina-4(2,1)-pyrrolidinacyclononaphane-1⁶-carboxamide, optionally substituted 1-((3S,4R)-4-amino-3-methyl-2-oxa-8-azaspiro[4.5]decan-8-yl)-7-methoxyisoquinoline-6-carboxamide, optionally substituted (S)-1-(3-(2-cyanoacetamido)piperidin-1-yl)-7-methoxyisoquinoline-6-carboxamide, optionally substituted (R)-7-methoxy-1-(piperidin-3-ylamino)isoquinoline-6-carboxamide, optionally substituted (R)-1-((1-(2-cyanoacetyl)piperidin-3-yl)amino)-7-methoxyisoquinoline-6-carboxamide, optionally substituted (R)-7-methoxy-1-(piperidin-3-ylthio)isoquinoline-6-carboxamide, optionally substituted (R)-1-((1-(2-cyanoacetyl)piperidin-3-yl)thio)-7-methoxyisoquinoline-6-carboxamide, optionally substituted (R)-7-methoxy-1-(piperidin-3-ylsulfonyl)isoquinoline-6-carboxamide, optionally substituted (R)-1-((1-(2-cyanoacetyl)piperidin-3-yl)sulfonyl)-7-methoxyisoquinoline-6-carboxamide, optionally substituted (R)-1-((1-(2-cyanoacetyl)pyrrolidin-3-yl)amino)-7-methoxyisoquinoline-6-carboxamide, optionally substituted (S)-1-(3-aminopiperidin-1-yl)-7-methoxy-4-(prop-1-yn-1-yl)isoquinoline-6-carboxamide, optionally substituted 1-(4-aminopiperidin-1-yl)-7-methoxy-4-(prop-1-yn-1-yl)isoquinoline-6-carboxamide, optionally substituted (S)-1-(3-aminopiperidin-1-yl)-4-bromo-7-methoxyisoquinoline-6-carboxamide, optionally substituted (R)-7-methoxy-1-(pyrrolidin-3-ylamino)isoquinoline-6-carboxamide, optionally substituted (S)-1-(3-aminopiperidin-1-yl)-4-cyano-7-methoxyisoquinoline-6-carboxamide, optionally substituted (R)-7-methoxy-1-(pyrrolidin-3-ylamino)isoquinoline-6-carboxamide, optionally substituted 7-methoxy-1-((((2S,3R)-3-methyl-5-oxopyrrolidin-2-yl)methyl)amino)-4-(prop-1-yn-1-yl)isoquinoline-6-carboxamide, optionally substituted 1-((((2S,3R)-3-ethyl-5-oxopyrrolidin-2-yl)methyl)amino)-7-methoxy-4-(prop-1-yn-1-yl)isoquinoline-6-carboxamide, optionally substituted 1-((3S,5S)-3-amino-5-ethylpiperidin-1-yl)-7-methoxy-4-(prop-1-yn-1-yl)isoquinoline-6-carboxamide, optionally substituted 1-((3S,5S)-3-amino-5-ethylpiperidin-1-yl)-7-methoxyisoquinoline-6-carboxamide, optionally substituted 1-((3S,4R)-3-amino-4-ethylpiperidin-1-yl)-7-methoxyisoquinoline-6-carboxamide, optionally substituted 1-((3S,4R)-3-amino-4-ethylpiperidin-1-yl)-7-methoxy-4-(prop-1-yn-1-yl)isoquinoline-6-carboxamide, optionally substituted 1-((((3S,4S)-3-ethyl-4-fluoro-5-oxopyrrolidin-2-yl)methyl)amino)-7-methoxyisoquinoline-6-carboxamide, optionally substituted 1-((((3S,4S)-4-fluoro-3-methyl-5-oxopyrrolidin-2-yl)methyl)amino)-7-methoxyisoquinoline-6-carboxamide, optionally substituted 1-((((3S,4S)-3-ethyl-4-fluoro-5-oxopyrrolidin-2-yl)methyl)amino)-7-methoxy-4-(prop-1-yn-1-yl)isoquinoline-6-carboxamide, or optionally substituted 1-((((3S,4S)-4-fluoro-3-methyl-5-oxopyrrolidin-2-yl)methyl)amino)-7-methoxy-4-(prop-1-yn-1-yl)isoquinoline-6-carboxamide.

90. A compound, or a pharmaceutically acceptable salt thereof, wherein the compound comprises: 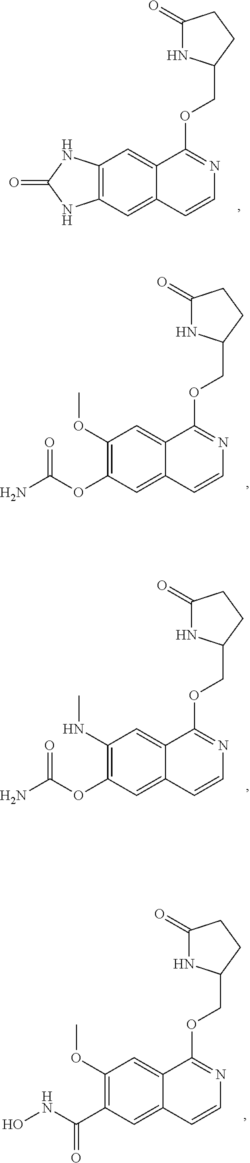 [SMILES: O=C1CCC(COc2nccc3cc4[nH]c(=O)[nH]c4cc23)N1 ; COc1cc2c(OCC3CCC(=O)N3)nccc2cc1OC(N)=O ; CNc1cc2c(OCC3CCC(=O)N3)nccc2cc1OC(N)=O ; COc1cc2c(OCC3CCC(=O)N3)nccc2cc1C(=O)NO] 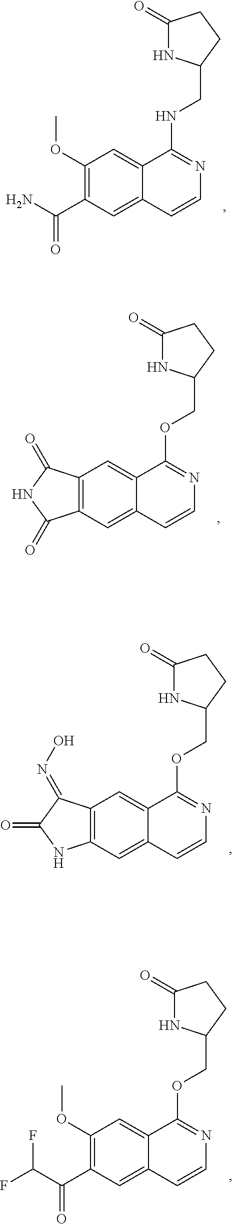 [SMILES: COc1cc2c(NCC3CCC(=O)N3)nccc2cc1C(N)=O ; O=C1CCC(COc2nccc3cc4c(cc23)C(=O)NC4=O)N1 ; O=C1CCC(COc2nccc3cc4c(cc23)/C(=N\O)C(=O)N4)N1 ; COc1cc2c(OCC3CCC(=O)N3)nccc2cc1C(=O)C(F)F] 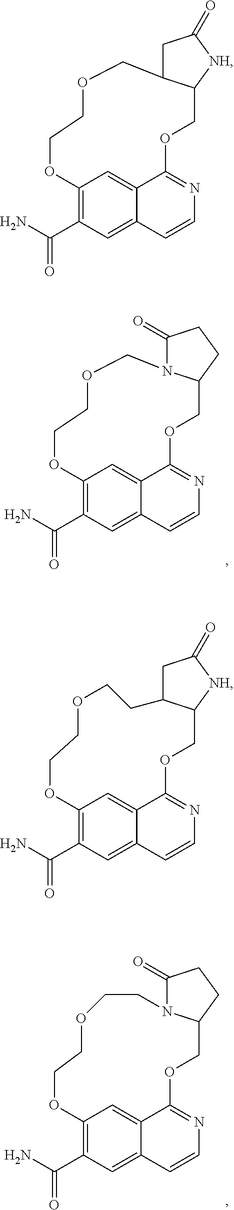 [SMILES: NC(=O)c1cc2ccnc3c2cc1OCCOCC1CC(=O)NC1CO3 ; NC(=O)c1cc2ccnc3c2cc1OCCOCN1C(=O)CCC1CO3 ; NC(=O)c1cc2ccnc3c2cc1OCCOCCC1CC(=O)NC1CO3 ; NC(=O)c1cc2ccnc3c2cc1OCCOCCN1C(=O)CCC1CO3] 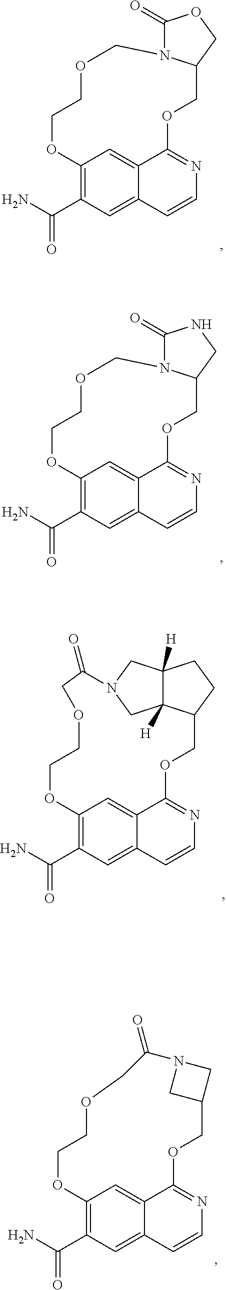 [SMILES: NC(=O)c1cc2ccnc3c2cc1OCCOCN1C(=O)OCC1CO3 ; NC(=O)c1cc2ccnc3c2cc1OCCOCN1C(=O)NCC1CO3 ; NC(=O)c1cc2ccnc3c2cc1OCCOCC(=O)N1C[C@@H]2CCC(CO3)[C@@H]2C1 ; NC(=O)c1cc2ccnc3c2cc1OCCOCC(=O)N1CC(CO3)C1] 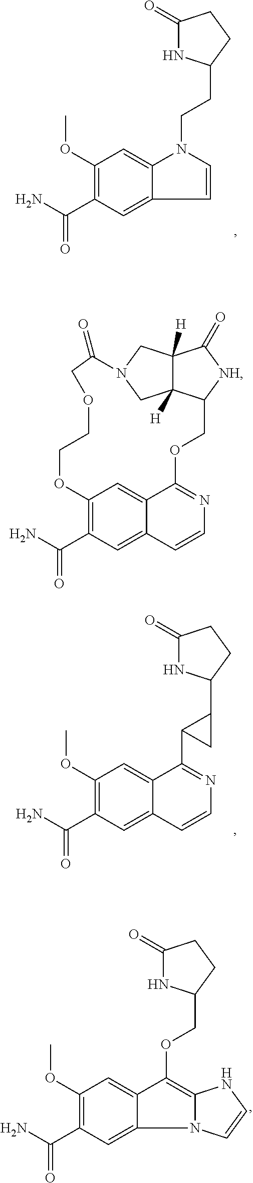 [SMILES: COc1cc2c(ccn2CCC2CCC(=O)N2)cc1C(N)=O ; NC(=O)c1cc2ccnc3c2cc1OCCOCC(=O)N1C[C@@H]2C(=O)NC(CO3)[C@@H]2C1 ; COc1cc2c(C3CC3C3CCC(=O)N3)nccc2cc1C(N)=O ; COc1cc2c(OCC3CCC(=O)N3)c3[nH]ccn3c2cc1C(N)=O] 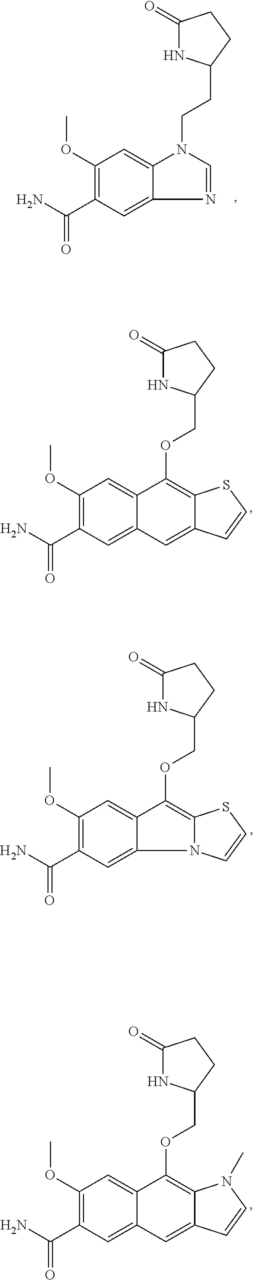 [SMILES: COc1cc2c(cc1C(N)=O)ncn2CCC1CCC(=O)N1 ; COc1cc2c(OCC3CCC(=O)N3)c3sccc3cc2cc1C(N)=O ; COc1cc2c(OCC3CCC(=O)N3)c3sccn3c2cc1C(N)=O ; COc1cc2c(OCC3CCC(=O)N3)c3c(ccn3C)cc2cc1C(N)=O] 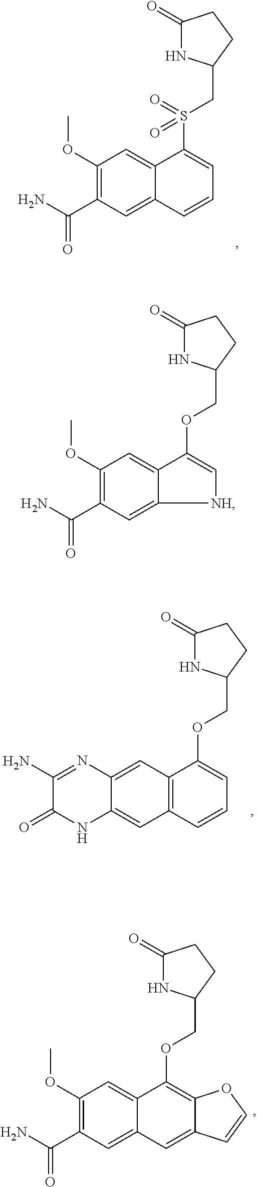 [SMILES: COc1cc2c(S(=O)(=O)CC3CCC(=O)N3)cccc2cc1C(N)=O ; COc1cc2c(OCC3CCC(=O)N3)c[nH]c2cc1C(N)=O ; Nc1nc2cc3c(OCC4CCC(=O)N4)cccc3cc2[nH]c1=O ; COc1cc2c(OCC3CCC(=O)N3)c3occc3cc2cc1C(N)=O] 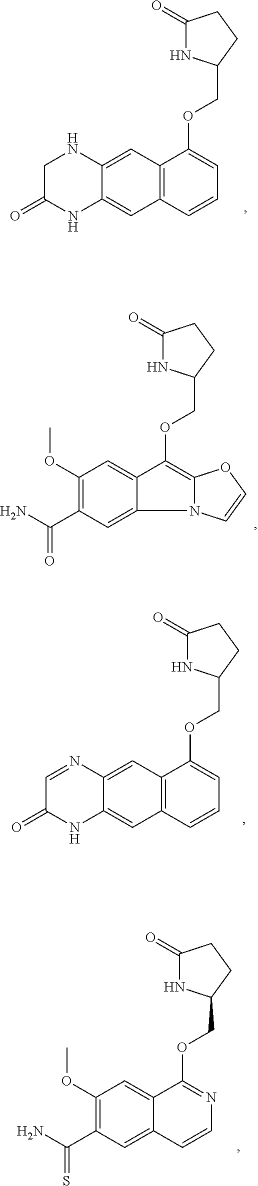 [SMILES: O=C1CNc2cc3c(OCC4CCC(=O)N4)cccc3cc2N1 ; COc1cc2c(OCC3CCC(=O)N3)c3occn3c2cc1C(N)=O ; O=C1CCC(COc2cccc3cc4[nH]c(=O)cnc4cc23)N1 ; COc1cc2c(OC[C@@H]3CCC(=O)N3)nccc2cc1C(N)=S] 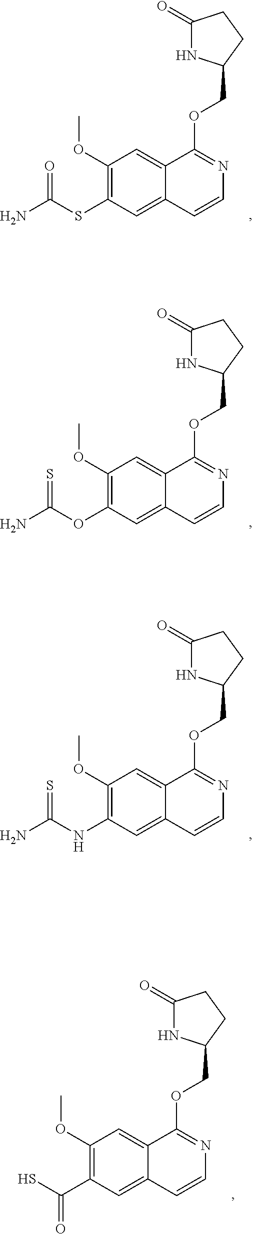 [SMILES: COc1cc2c(OC[C@@H]3CCC(=O)N3)nccc2cc1SC(N)=O ; COc1cc2c(OC[C@@H]3CCC(=O)N3)nccc2cc1OC(N)=S ; COc1cc2c(OC[C@@H]3CCC(=O)N3)nccc2cc1NC(N)=S ; COc1cc2c(OC[C@@H]3CCC(=O)N3)nccc2cc1C(=O)S] 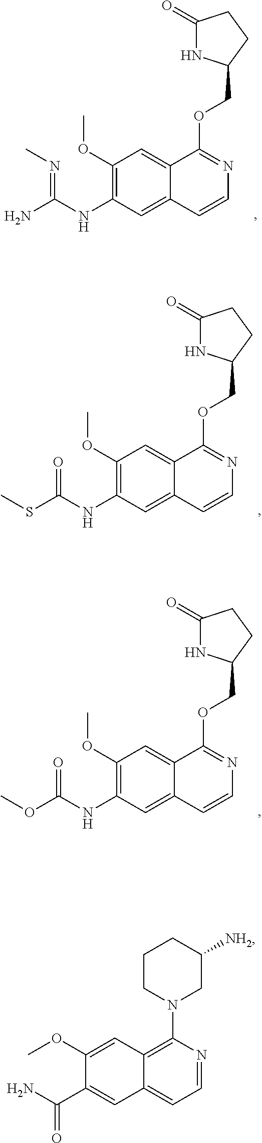 [SMILES: C/N=C(\N)Nc1cc2ccnc(OC[C@@H]3CCC(=O)N3)c2cc1OC ; COc1cc2c(OC[C@@H]3CCC(=O)N3)nccc2cc1NC(=O)SC ; COC(=O)Nc1cc2ccnc(OC[C@@H]3CCC(=O)N3)c2cc1OC ; COc1cc2c(N3CCC[C@H](N)C3)nccc2cc1C(N)=O] 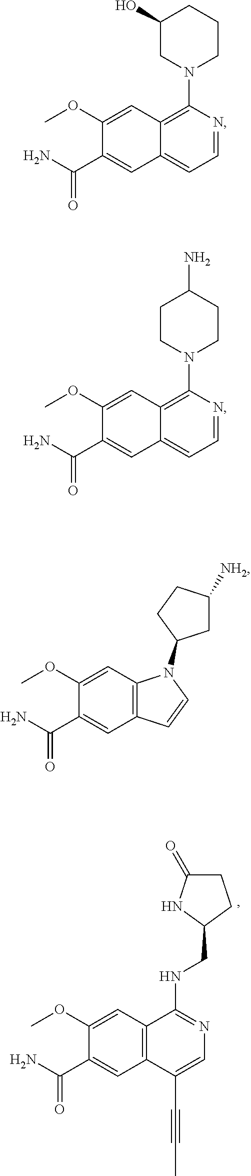 [SMILES: COc1cc2c(N3CCC[C@H](O)C3)nccc2cc1C(N)=O ; COc1cc2c(N3CCC(N)CC3)nccc2cc1C(N)=O ; COc1cc2c(ccn2[C@H]2CC[C@H](N)C2)cc1C(N)=O ; CC#Cc1cnc(NC[C@@H]2CCC(=O)N2)c2cc(OC)c(C(N)=O)cc12] 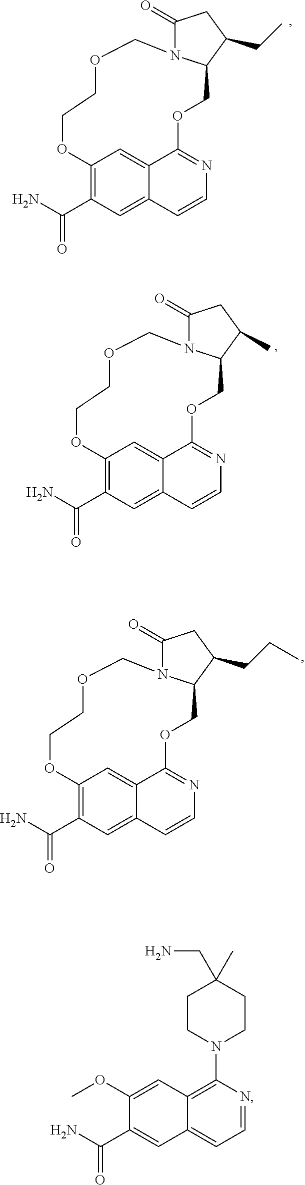 [SMILES: CC[C@@H]1CC(=O)N2COCCOc3cc4c(nccc4cc3C(N)=O)OC[C@H]12 ; C[C@@H]1CC(=O)N2COCCOc3cc4c(nccc4cc3C(N)=O)OC[C@H]12 ; CCC[C@@H]1CC(=O)N2COCCOc3cc4c(nccc4cc3C(N)=O)OC[C@H]12 ; COc1cc2c(N3CCC(C)(CN)CC3)nccc2cc1C(N)=O] 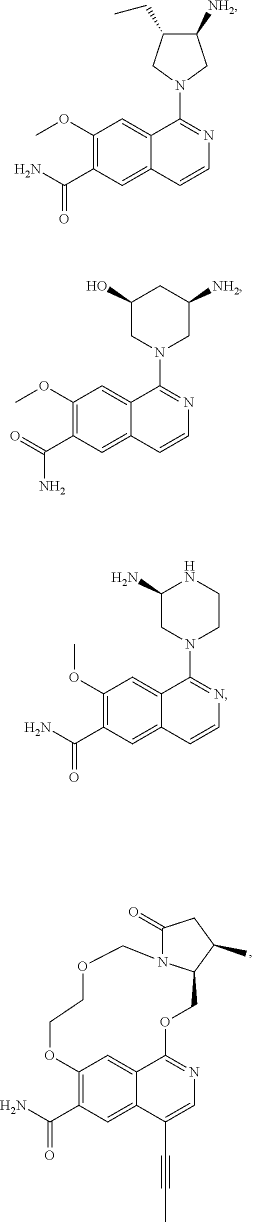 [SMILES: CC[C@H]1CN(c2nccc3cc(C(N)=O)c(OC)cc23)C[C@@H]1N ; COc1cc2c(N3C[C@H](N)C[C@H](O)C3)nccc2cc1C(N)=O ; COc1cc2c(N3CCN[C@H](N)C3)nccc2cc1C(N)=O ; CC#Cc1cnc2c3cc(c(C(N)=O)cc13)OCCOCN1C(=O)C[C@@H](C)[C@H]1CO2] 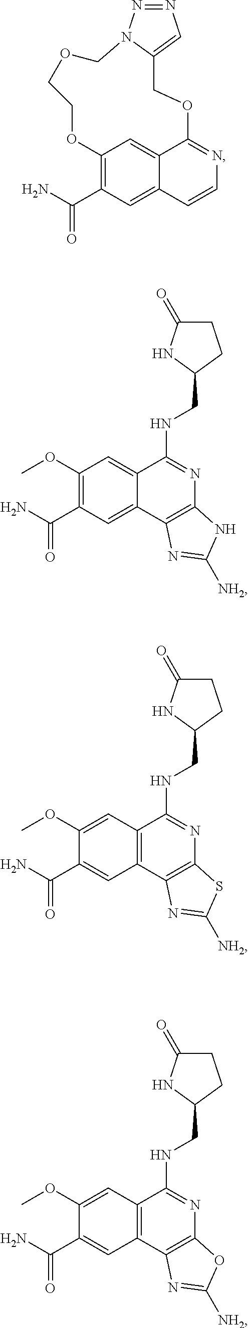 [SMILES: NC(=O)c1cc2ccnc3c2cc1OCCOCn1nncc1CO3 ; COc1cc2c(NC[C@@H]3CCC(=O)N3)nc3[nH]c(N)nc3c2cc1C(N)=O ; COc1cc2c(NC[C@@H]3CCC(=O)N3)nc3sc(N)nc3c2cc1C(N)=O ; COc1cc2c(NC[C@@H]3CCC(=O)N3)nc3oc(N)nc3c2cc1C(N)=O] 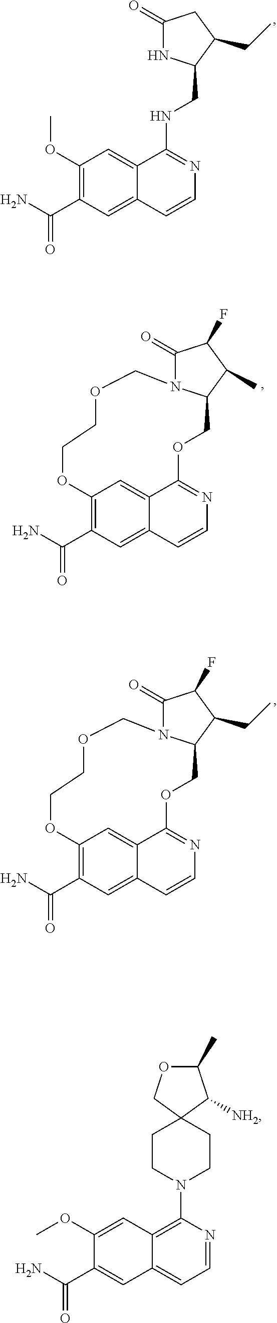 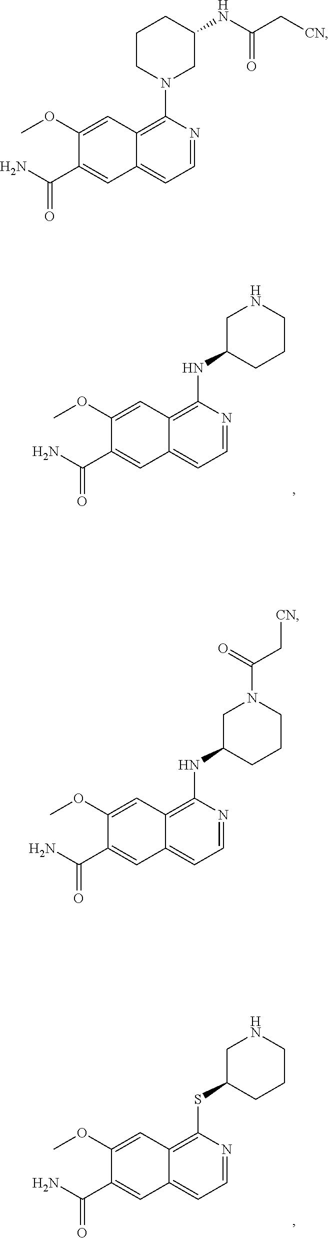 [SMILES: COc1cc2c(N3CCC[C@H](NC(=O)CC#N)C3)nccc2cc1C(N)=O ; COc1cc2c(N[C@@H]3CCCNC3)nccc2cc1C(N)=O ; COc1cc2c(N[C@@H]3CCCN(C(=O)CC#N)C3)nccc2cc1C(N)=O ; COc1cc2c(S[C@@H]3CCCNC3)nccc2cc1C(N)=O] 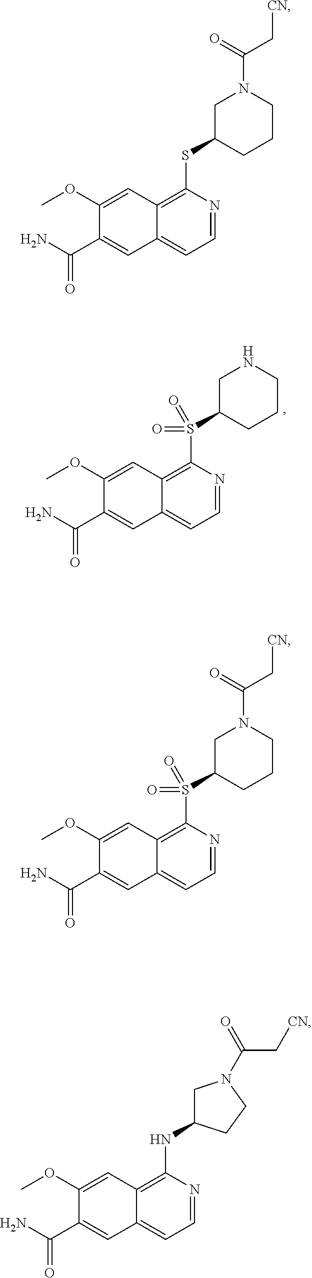 [SMILES: COc1cc2c(S[C@@H]3CCCN(C(=O)CC#N)C3)nccc2cc1C(N)=O ; COc1cc2c(S(=O)(=O)[C@@H]3CCCNC3)nccc2cc1C(N)=O ; COc1cc2c(S(=O)(=O)[C@@H]3CCCN(C(=O)CC#N)C3)nccc2cc1C(N)=O ; COc1cc2c(N[C@@H]3CCN(C(=O)CC#N)C3)nccc2cc1C(N)=O] 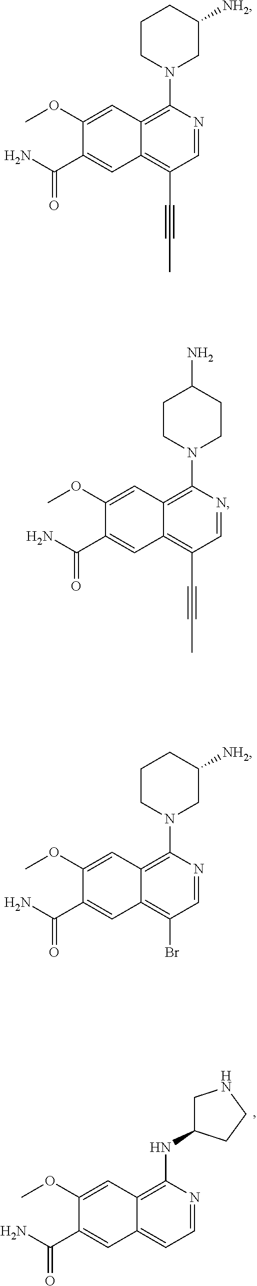 [SMILES: CC#Cc1cnc(N2CCC[C@H](N)C2)c2cc(OC)c(C(N)=O)cc12 ; CC#Cc1cnc(N2CCC(N)CC2)c2cc(OC)c(C(N)=O)cc12 ; COc1cc2c(N3CCC[C@H](N)C3)ncc(Br)c2cc1C(N)=O ; COc1cc2c(N[C@@H]3CCNC3)nccc2cc1C(N)=O] 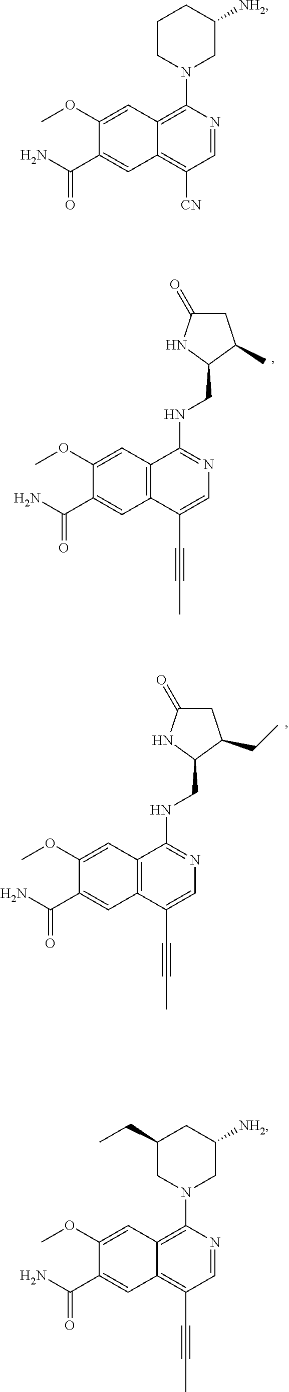 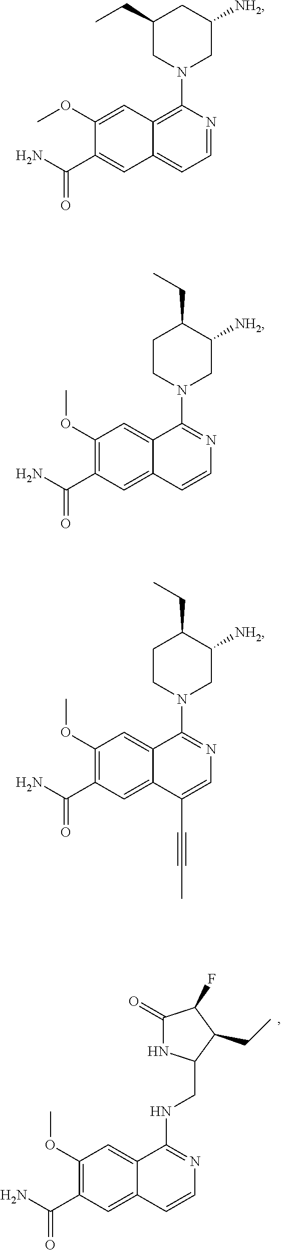 [SMILES: CC[C@H]1C[C@H](N)CN(c2nccc3cc(C(N)=O)c(OC)cc23)C1 ; CC[C@@H]1CCN(c2nccc3cc(C(N)=O)c(OC)cc23)C[C@H]1N ; CC#Cc1cnc(N2CC[C@@H](CC)[C@H](N)C2)c2cc(OC)c(C(N)=O)cc12 ; CC[C@H]1C(CNc2nccc3cc(C(N)=O)c(OC)cc23)NC(=O)[C@H]1F] 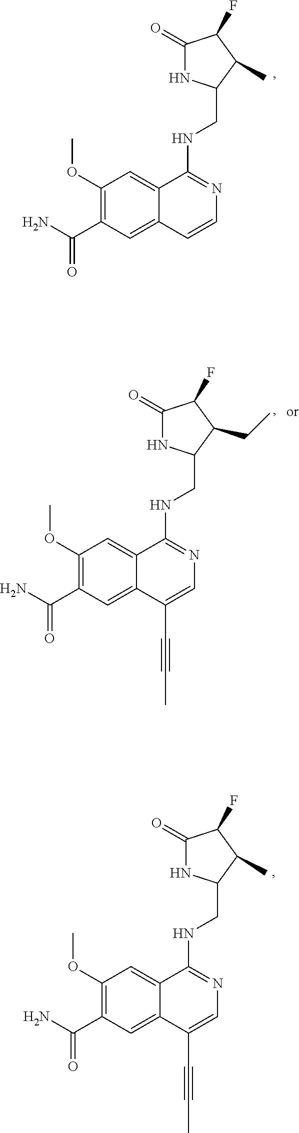 [SMILES: COc1cc2c(NCC3NC(=O)[C@@H](F)[C@H]3C)nccc2cc1C(N)=O ; CC#Cc1cnc(NCC2NC(=O)[C@@H](F)[C@H]2CC)c2cc(OC)c(C(N)=O)cc12 ; CC#Cc1cnc(NCC2NC(=O)[C@@H](F)[C@H]2C)c2cc(OC)c(C(N)=O)cc12]
wherein each structure is optionally substituted.

91. The compound of any preceding claim, wherein the compound is an R-enantiomer.

92. The compound of any preceding claim, wherein the compound is an S-enantiomer.

93. The compound of any preceding claim, wherein the compound is a single diastereomer.

94. The compound of any preceding claim, wherein the compound is deuterated.

95. The compound of any preceding claim, wherein any single substituent of any optionally substituted moiety has a molecular weight of 15 Da to 200 Da.

96. The compound of claim 1, wherein A contains an optionally substituted aromatic all carbon ring which has an R¹ substituent.

97. A pharmaceutical composition comprising a compound of any one of claims 1-96 or a pharmaceutically acceptable salt thereof or a tautomer of said compound or said salt and a pharmaceutically acceptable vehicle, diluents or carrier.

98. A method of treating a mammal, including a human, having a disease or condition related to IRAK4-mediated disorders including cancer, autoimmune diseases; inflammatory diseases; or autoinflammatory conditions, comprising administering to a mammal in need thereof a therapeutically effective amount of a compound of any one of claims 1-96.

99. Use of a compound of any one of claims 1-96 in the manufacture of a medicament for the treatment of cancer, autoimmune diseases; inflammatory diseases; autoinflammatory conditions related to IRAK4-mediated disorders in a mammal.

100. A method of treating cancer and other IRAK4-mediated diseases or disorders comprising administering a pharmaceutical composition of claim 97, to a mammal in need thereof.

101. A process for making a pharmaceutical composition comprising combining a compound of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96, and at least one pharmaceutically acceptable carrier.
